(19) United States
(12) Patent Application Publication
(10) Pub. No.: US 2023/0296622 A1
(43) Pub. Date: Sep. 21, 2023

(54) COMPOSITIONS AND METHODS OF PREDICTING TIME TO ONSET OF LABOR

(71) Applicant: The Board of Trustees of the Leland Stanford Junior University, Stanford, CA (US)

(72) Inventors: Brice L. Gaudilliere, Palo Alto, CA (US); Ina Stelzer, Redwood City, CA (US); Xiaoyuan Han, Redwood City, CA (US); Nima Aghaeepour, Mountain View, CA (US); Martin S. Angst, Palo Alto, CA (US); Sajjad Ghaemi, Redwood City, CA (US); Julien Hedou, San Francisco, CA (US)

(21) Appl. No.: 18/019,228

(22) PCT Filed: Aug. 17, 2021
(86) PCT No.: PCT/US2021/046312

§ 371 Date: Feb. 1, 2023

Related U.S. Application Data
(60) Provisional application No. 63/066,708, filed on Aug. 17, 2020.

(51) Int. Cl. G01N 33/68; G01N 33/74

(52) U.S. Cl. CPC G01N 33/689; G01N 33/74; G01N 2440/14; G01N 2570/00

(57) ABSTRACT

Multiparametric analysis is performed at the single cell level of biological samples obtained from an individual during pregnancy to obtain a determination of changes in the interactome, integrating metabolome, immunome and proteome features during pregnancy that are predictive of time to onset of labor.

### CROSS REFERENCE TO RELATED APPLICATION

The present application claims the benefit of and priority to U.S. Provisional Pat. Application No. 63/066,708 filed Aug. 17, 2020, the entire disclosure of which is hereby.

### BACKGROUND OF THE INVENTION

During a human pregnancy, the onset of labor is precisely timed to ensure the delivery of a healthy newborn. However, what determines the timing of parturition in human pregnancy is not clearly understood. The ability to accurately predict the onset of labor is of high clinical importance as preterm (< 37 weeks of gestation) or post-term (> 42 weeks of gestation) deviations are associated with complications for the mother and her offspring. Preterm labor is the most common cause of childhood mortality below the age of five years worldwide. Post-term labor doubles both the risk for perinatal mortality as well as the rate of maternal complications compared to that of term delivery.

Existing methods for predicting the onset of labor perform poorly. In current clinical practice, the Estimate Day of Delivery (EDD) is calculated based on an estimate of gestational age (GA) from the time of the last menstrual period (LMP). The GA and EDD is further determined by the first accurate ultrasound examination. While useful for determining GA, these methods estimate the delivery date based on GA and a presumed duration of 40 weeks of gestation, leading to inaccurate predictions of labor onset as most pregnancies deviate from this norm. Novel estimation approaches including predictive biomarkers are critically needed to better predict the onset of labor in healthy and pathological pregnancies that will benefit clinical management decisions.

A comprehensive characterization of the biological processes that precede the spontaneous onset of labor is a key step for the identification of predictive biomarkers of labor onset. The maintenance of pregnancy relies on finely-tuned endocrine, metabolic, and immunologic adaptations, which are readily detectable in maternal blood using high-content metabolomic, proteomic, and cytomic technologies. At the onset of labor, a major transition occurs in the fetomaternal physiology that culminates in the delivery of the fetus, including the breakdown of fetomaternal immune tolerance by immune infiltration into fetal membranes and the placenta, endocrine changes, rupture of fetal membranes, cervical dilation, and augmentation of uterine contractility.

The timing of systemic molecular and cellular events that mark the transition from pregnancy maintenance to parturition, which begins with spontaneous rupture of membranes and/or labor contractions, is ill-defined. Prior studies have provided important information with regard to systemic maternal adaptations that track GA during pregnancy. However, to understand the biological transition to labor, studies are needed that specifically examine the timing of spontaneous labor onset as a primary outcome, rather than an outcome dependent upon assessment of GA. Thus far, limitations in study design - e.g. inclusion of medically-induced labor cases - or technological limitations - e.g. the limited coverage of immune-system wide adaptations on a single-cell level - have precluded a comprehensive analysis of metabolomic, proteomic, and immunologic events predictive of the spontaneous onset of labor.

The present disclosure addresses this issue.

### SUMMARY OF THE INVENTION

Compositions and methods are provided for blood-based classification, diagnosis, prognosis, theranosis, and/or prediction during pregnancy for timing of labor onset, where the prediction of timing is made during pregnancy, prior to the onset of labor. The data provided herein demonstrates a precisely timed transition from pregnancy maintenance to pre-labor biology, specified as coordinated dynamics in “features” such as steroid hormone metabolism, placental biology, fetal membrane activation, and innate immune regulation that are intimately linked to the time to labor. The analysis and prediction of labor onset is used to guide therapeutic approaches to extend pregnancy when the labor onset signature is detected early, preterm birth, or to accelerate labor processes to avoid the need for induction of labor in post-date pregnancies. Specifically, fluctuations in the parameters (features) demonstrate a marked transition from pregnancy progression to pre-labor biology two to four weeks before delivery.

In some embodiments, a set of features selected from plasma metabolites, plasma proteins, circulating immune cells; and circulating immune cell responses are assessed at two or more timepoints to project the time to onset of labor, by determining changes over time. In some embodiments one or more of mass spectroscopy, protein assays (aptamer-based or antibody-based detection of proteins), high-dimensional mass cytometry, and fluorescence-based flow cytometry immunoassay are used to characterize the dynamic changes in maternal blood during pregnancy.

Analysis may be performed at any time during pregnancy, e.g. during the first trimester to predict very pre-term births. In other embodiments analysis is performed, for example, after about 20 gestational weeks, after about 22 gestational week, after about 25 gestational weeks, after about 28 gestational weeks, after about 30 gestational weeks, after about 32 gestational weeks, and usually before full term, e.g. prior to about 40 gestational weeks. In some embodiments analysis is performed on samples taken during the third trimester at periods of from about weekly, bi-we.

As discussed herein, change over time in blood markers can be an important indicator, e.g. with at least 2, 3, 4, 5, 6, 7, 8, 9, 10 or more different time points for assessment, which time points may be 2, 3, 4, 5, 6, 7, 8, 9, 10, 11, 12, 13, 14, 15, or more weeks apart, for example with a sample taken before and after gestational week 25, week 26, week 27, week 28, week 29, week 30, week 31, week 32, week 33, week 34, week 35, week 36, week 37, week 38, week 39, week 40 or more.

A significant change in a marker between two time points can be a rate of change from about |0.01|, |0.02|, up to |0.05|, where a decreasing value will be, for example, -0.01, -0.02, -0.05, etc., where the time points are from 2-4 weeks apart, 2-4 months apart, and can be around 3 months (one trimester) apart.

Individuals showing a set of changes indicative of pre-term labor or post-term labor can be treated accordingly. Because the diagnosis can be provided significantly before clinical symptoms, the methods herein provide a means of timely intervention. Intervention can include monitoring blood pressure at regular, e.g., daily, intervals, inducing labor, bed rest, and the like.

The predictive analysis provided herein utilizes a multivariate model that accurately times the onset of labor. A stacked generalization (SG) algorithm is applied to a high dimensional multi-omic dataset for an integrated model that accurately predicts time for the onset of labor. Multivariate Least Absolute Shrinkage and Selection Operator (LASSO) linear regression models were first individually built for each omic dataset, then integrated into a single model by SG. An advantage of the SG method is that differences in size and modularity of individual omic modalities are accounted for to prevent datasets of higher dimensions (e.g., metabolome) to overwhelm the integrated model. The SG model predicted the time to labor from the measurement of metabolic, proteomic, and immunologic features with high accuracy. The results indicate that assessment of maternal circulating factors in the peripheral blood provided an accurate prediction for the timing of labor onset that was independent of an estimate of GA.

The features for analysis comprise at least 1, at least 5, at least 8, at least 10, at least 15, at least 20, at least 25, at least 30, at least 35, at least 40 and up to 45; and may be not more than 40, not more than 35, not more than 30, not more than 25, not more than 20,not more than 15; and in some embodiments from 5-15 or from 5-10 of the features selected from:

| | | |
|---|---|---|
| metabolome | 331.2264_8.4 (17-OHP/P4 derivative) | acceleration/increase |
| metabolome | 331.2264_8.1 (17-OHP/P4 derivative) | acceleration/increase |
| metabolome | 331.2265_8.9 (17-OHP/P4 derivative) | constant |
| metabolome | 361.2017_7.1 (Cortisol) | constant |
| metabolome | 415.3204_12 (C₂₇H₄₂O₃) | constant |
| metabolome | 151.0615_2.6 (1-Methylhypoxanthine) | constant |
| metabolome | 411.1844 8.7 (17-OH pregnenolone sulfate) | acceleration/increase |
| metabolome | 193.0618_5.3 (4-Aminohippuric acid) | constant |
| metabolome | 151.0612_6 (Arabitol, Xylitol) | constant |
| metabolome | 219.0774_6.3 (5-Hydroxytryptophan) | constant |
| metabolome | 236.09294.3 (N-Lactoylphenylalanine) | constant |
| metabolome | 397.205_10.6 (6 (Pregnanolone sulfate) | deceleration/decrease |
| proteome | IL-1R4 | acceleration/increase |
| proteome | Plexin-B2 (PLXB2) | constant |
| proteome | Discoidin domain receptor 1 (DDR1) | constant |
| proteome | Angiopoietin-2 | constant |
| proteome | Vascular Endothelial Growth Factor 121 | constant |
| proteome | Cystatin C | acceleration/increase |
| proteome | SLIT and NTRK-like protein 5 (SLTRK5) | deceleration/decrease |
| proteome | Secr. Leukocyte Peptidase Inhibitor (SLPI) | acceleration/increase |
| proteome | Activin A | constant |
| proteome | Antithrombin III | acceleration/decrease |
| proteome | Macrophage inhibitory cytokine-1 (MIC-1) | constant |
| proteome | Siglec-6 | acceleration/increase |
| proteome | urokinase-type Plasminogen Activator (uPA) | constant |
| proteome | Matrix Metalloproteinase (MMP) 12 | constant |
| proteome | Soluble tunica interna endothelial cell kinase (sTie)-2 | deceleration/decrease |
| proteome | LAG3 | constant |
| proteome | Endostatin | constant |
| proteome | GA733-1 protein | deceleration/decrease |
| immunome | CD69⁻CD56^{lo}CD16⁺NK, pSTAT1, IFNα | deceleration/increase |
| immunome | Granulocytes (freq) | constant |
| immunome | CD69⁺CD56^{lo}CD16⁺NK, pSTAT1, IFNα | deceleration/increase |
| immunome | CD62L⁺CD4Tₙₐᵢᵥₑ, pMAPKAPK2, IFNα | deceleration/increase |
| immunome | ncMC, pCREB, GM-CSF | acceleration/decrease |
| immunome | CD69⁺CD8Tₘₑₘ, pMAPKAPK2, basal | acceleration/decrease |
| immunome | pDC, pSTAT1, IFNα | deceleration/increase |
| immunome | B cells, pMAPKAPK2, LPS | deceleration/increase |
| immunome | CD4Tem, pMAPKAPK2, basal | acceleration/decrease |
| immunome | CD69⁺CD8T mem, pMAPKAPK2, IFNα | deceleration/increase |
| immunome | B cells (freq) | constant |
| immunome | CCR5⁺CCR⁺CD4Tₑₘ, pNFKB, IL-2,4,6 | deceleration/decrease |
| immunome | CCR⁺CCR2⁺CD4T_{cm}, IκB, basal | acceleration/decrease |
| immunome | DC, pSTAT6, IFNα | acceleration/decrease |
| immunome | DC, pMAPKAPK2, basal | acceleration/decrease |

Overall, the multi-modal analysis of plasma analytes and peripheral blood immune cells during the 100 days prior to the onset of labor revealed a concerted behavior between the metabolomic, proteomic, and immunologic systems over time, or interactome, as pregnancy progresses towards labor.

Metabolic trajectories highlighted biological processes progressing linearly throughout the last 100 days of pregnancy until labor. The plasma concentration of cortisol increased steadily from day -100 before labor to time of labor. Among the most informative surging metabolomic features were isomers of 17-hydroxyprogesterone (17-OHP) and 17-hydroxypregnenolone sulfate, an upstream substrate for the production of 17-OHP. Plasma concentrations of these features increased in accelerated fashion within the last 30 days before the day of labor. Our data provide additional temporal information showing that a surge in 17-OHP, one of the most informative features of the predictive model, is tightly linked to the timing of labor. Furthermore, levels of pregnenolone sulfate showed decelerating behavior, stagnating around 30 days before the day of labor.

Among the most informative proteomic features of the predictive model were five features with accelerating or decelerating patterns that pointed towards important pre-labor fluctuations with respect to placental biology, coagulation, and inflammation. The most informative degree 2a proteomic feature was IL-1 receptor type 4 (IL-1R4), the soluble inhibitory receptor of the pro-inflammatory cytokine IL-33. IL-1R4 plasma levels surged during the last 30 days before labor, and can be an important sensor of inflammation during the late phase of pregnancy. Other features that surged with approaching labor were two proteins highly expressed by the placenta, Activin-A and Sialic Acid Binding Ig Like Lectin (Siglec)-6. The trajectory of antithrombin III (ATIII) negatively accelerated during the last 30 days before the onset of labor. Soluble tunica interna endothelial cell kinase (sTie)-2 displayed a decelerating trajectory. Fetal-membrane-derived PLXB2 and DDR1 had constantly rising levels. The coordinated trajectories of angiogenic factors sTie2, Angiopoietin-2, vascular endothelial growth factor (VEGF)121, Activin-A, and Siglec-6 are integral components of a plasma fetoplacental signature that portends the impending onset of labor.

Immune cell trajectories predominantly followed a decelerating pattern in contrast to accelerating or constantly increasing plasma factor trajectories. Decelerating immune cell trajectories were observed along the Janus kinase (JAK)-signal transducer and activator of transcription (STAT) and MyD88 signaling pathways in both innate and adaptive immune cells. This decelerating behavior was particularly pronounced in innate immune cells, as illustrated by the phosphorylated (p)STAT1 signal in CD56^{dim}CD16⁺ NK cells and the pSTAT6 signal in dendritic cells (DCs) in response to IFNα, the pP38, pERK and pCREB signals in classical monocytes (cMCs) in response to LPS and GM-CSF, and the pCREB response in non-classical monocytes (ncMCs) in response to GM-CSF. During the 100 days preceding labor, these pro-inflammatory innate immune cell responses first increased, then stagnated or decreased closer to the onset of labor. There is a regulated dampening of systemic immune cell responses before labor onset to counteract the anticipated pro-inflammatory environment occurring during labor and parturition.

The omnipresence of degree 2 trajectories across all omic datasets shows a period of disruption with approaching labor that reverberated across all measured biological systems. Identifying the timing of such non-linear transition is clinically relevant as it defines when the assessment of peripheral blood analytes is linked to pre-labor biology rather than a reflection of the biology relevant for the progression of pregnancy. A piece-wise fused LASSO regression analysis combines the predictions rho (ρ) of two LASSO regression models built using the data points before or after a given DOL threshold, while varying the threshold across all time points. A maximum ρ value is reached when the models on each side of the threshold contain distinct biological features that, when combined, reach maximal predictive accuracy. The piece-wise fused LASSO regression analysis produced a maximum at 23 days before labor onset demarcating a transition when the DOL is best estimated using two distinct biological models.

The model in FIG. 5C summarizes major characteristics of the biology before and after the transition period occurring 2-4 weeks before labor. There is a shift in systemic immune responses from increasing immune responsiveness to the regulation of inflammatory responses, most prominently shown in Jak-STAT and MyD88 responses in NK cells, DCs, and MC subsets. These pre-labor immune adaptations are paralleled by a transition in the cytokine and endocrine environment characterized by accelerating proteomic and metabolomic trajectories, which are prominently evident in levels of 17-OHP and IL-1R4.

In one embodiment of the invention, the methods of determining time to labor in a patient during pregnancy comprises obtaining a patient sample(s) comprising circulating immune cells. Blood samples are a convenient source of circulating immune cells, particularly whole blood, although PBMC fractions also find use. Blood or plasma samples are also used for determination of the presence of plasma proteins, and metabolites. The patient cell sample is optionally stimulated ex vivo with an effective dose of an agent that stimulates pSTAT1 or pSTAT5, e.g. IFNα, or IL-2 although as shown herein basal levels can be sufficiently informative. The sample(s) is physically contacted with a panel of affinity reagents specific for signaling proteins and for markers that distinguish subsets of immune cells. Usually the affinity reagents comprise a detectable label, e.g. isotope, fluorophore, etc. Signal intensity of the markers is measured, preferably at a single cell level. Suitable methods of analysis include, without limitation, flow cytometry, mass cytometry, confocal microscopy, and the like. The data, which can include measurements of intensity of signaling molecules and changes in phosphorylation in selected immune cell subsets, etc., is compared to measurements of the same from the baseline cell population. The data can be normalized for comparison.

In other embodiments of the invention a device or kit is provided for the analysis of patient samples. Such devices or kits will include reagents that specifically identify one or more cells, plasma proteins and metabolites indicative of the status of the patient, including without limitation affinity reagents. The reagents can be provided in isolated form, or pre-mixed as a cocktail suitable for the methods of the invention. A kit can include instructions for using the plurality of reagents to determine data from the sample; and instuctions for statistically analyzing the data. The kits may be provided in combination with a system for analysis, e.g. a system implemented on a computer. Such a system may include a software component configured for analysis of data obtained by the methods of the invention.

Also described herein is a method for assessing time to onset of labor during pregnancy, comprising: obtaining a dataset associated with a sample obtained from the subject, wherein the dataset comprises quantitiative data from the markers disclosed herein and analyzing the dataset for changes of these markers, wherein a statistically significant match with a model disclosed herein is indicative of the time to onset of labor. The data may be analyzed by a computer processor. The processor may be communicatively coupled to a storage memory for analyzing the data. Also described herein is a computer-readable storage medium storing computer-executable program code, the program code comprising: program code for storing and analyzing data obtained by the methods of the invention.

In an embodiment, the method further comprises selecting a treatment regimen for the patient based on the analysis. Treatment regimens of interest may include, without limitation, decision-making for proceeding with bed rest, extended hospital stay, medication for hypertension, blood-pressure monitoring, low dose aspirin, low dose IL-2, extended care at an intermediate facility, increased follow-up, and the like for an indication of pre-term labor.

For an indication of post-term labor treatment may include, without limitation, administration of misoprostol; of oxytocin; of dinoprostone; inducing labor with a balloon catheter, sweeping membranes; rupture of membranes, and the like.

### BRIEF DESCRIPTION OF THE DRAWINGS

FIG. 1. The maternal metabolome, proteome, and immunome were assessed during the 100-day period preceding the day of labor. (A) Peripheral blood was obtained serially from 63 women during the 100 days preceding spontaneous labor. The primary outcome of the analysis was the time to labor (TL), such that the prediction of the day of labor did not consider estimates of GA. Raster plots depicting the day of sampling for the training (top plot; N = 53, n = 150 samples) and test (bottom plot; N = 10, n = 27 samples) cohort, and the TL distribution (range [-112, 0]), calculated as the difference between the day of labor (TL 0, red line) and the day of sampling (filled dots). At least one sample was collected on any day of the 100 days preceding the day of labor (cumulative count plot). (B) Plasma samples were used in the analysis of the circulating metabolome (high-throughput mass spectrometry) and proteome (aptamer-based technology). Whole-blood samples were used in the analysis of the systemic immunome (mass cytometry). In total, 7142 features were generated per sample from all three datasets and integrated into a multivariate model to predict the TL.

FIG. 2. The late-gestational maternal interactome highlights interconnectivity between biological systems. (A to C) Intraomic correlation networks of metabolome, proteome, and immunome features during the 100 days preceding labor in the training cohort (N = 53). Each node represents a biological feature. Correlations between features are represented by edges. Red/blue nodes highlight features positively/negatively correlated with the TL. Dot size indicates the -log10 of P value of the correlation (Spearman). Clusters of features most highly correlated with the TL are shaded in gray and annotated. (D) Distributions of all correlations within (intraomic) and between (interomic) modalities in the original as well as simulated random datasets. The false discovery rate (FDR) threshold of 0.05 was computed from the generated distribution of random features in a target-to-decoy approach to filter the correlations with FDR > 0.05, corresponding to an absolute (|x|) correlation coefficient cutoff at 0.46. (E) Chord diagram of interomic (between-dataset) correlations between metabolome, proteome, and immunome features in the last 100 days before the day of labor. The outer circle represents all features with FDR-adjusted absolute correlation coefficients [Spearman R (0.46, 1.0), FDR < 0.05], colored by the respective biological modality. Shaded inner connections represent interomic correlations between the metabolome, proteome, and immunome as specified by color codes. The number of FDR-adjusted interactions between two omics is visualized as normalized to the number of total possible interomic interactions. (F) Quantification of the number of interomic interactions visualized in (E). The number of interomic correlations between the three biological modalities divided into weak (0.46 to 0.6), moderate (0.6 to 0.8), and strong (0.8 to 1.0) absolute correlation coefficients is shown.

FIG. 3. Multiomic modeling of the maternal interactome predicts labor onset. (A) Integration of all three modalities (metabolome, proteome, and immunome) using a stacked generalization (SG) method. (B and C) Regression of predicted versus true TL (days) derived from the SG model [training cohort, Pearson R = 0.85, 95% CI [0.79 to 0.89], P = 1.2 × 10^-40, RMSE = 17.7 days, N = 53 patients (B); test cohort, Pearson R = 0.81, 95% CI [0.61 to 0.91], P = 3.9 × 10^-7, RMSE = 17.4 days, N = 10 patients (C)]. (D) Volcano plot depicting the 45 most informative SG model features in the training cohort. Feature importance to the overall predictive model is plotted on the x axis (SG model coefficient), correlation with the TL is plotted on the y axis [-Log10 (P value)]. Orange colors depict positive correlations with the TL, and teal colors depict negative correlations. See table 4 for number-to-feature key. (E) Pathway enrichment analysis was performed on metabolic and proteomic top SG model features (see Materials and Methods; P values derived from hypergeometric and Fisher’s test). All 45 most informative model features are depicted in a correlation network to visualize interomic correlations (edges indicate an absolute R > 0.46, N = 53). See also FIG. 4, FIG. 6, and table 4.

FIG. 4. Trajectories of the maternal metabolome, proteome, and immunome reveal alterations in prelabor dynamics. (A) Distribution of relevance-of-fit P values for the trajectories assigned to SG model features in comparison to nonselected features demonstrates goodness of fit of curve classification (N = 53 patients, n = 150 samples). Feature trajectories were classified as linear or quadratic on the basis of the goodness of fit with Akaike information criterion and relevance of fit with associated P value (F statistic). Degree 1 (B to D), degree 2a (E to G), or degree 2b (H to J) trajectories are plotted over time for the metabolome (left), proteome (middle), and immunome (right). Lines represent smoothened spline (df = 3, Z-scored) for all features. The most informative model features are highlighted and numbered (in reference to FIG. 3D and table 4). A representative feature is shown (inset) for each trajectory type including its correlation with TL (Spearman coefficient [95% Cl], and associated P value). (K) Radar plot quantifying the distribution of degree 1 (linear), degree 2a [quadratic, accelerating (surging of an increasing or decreasing pattern over time)], and degree 2b [quadratic, decelerating (plateauing of an increasing or decreasing pattern over time)] trajectories among all multiomic features. See also FIGS. 7 to 12 and tables 4 and 5.

FIG. 5. A breakpoint in omic trajectories demarcates the transition from pregnancy maintenance to prelabor biological adaptations. (A) Schematic of a piecewise fused LASSO regression combining predictions rho (ρ) of two regression models built from all datasets before and after a particular TL threshold, while sliding the threshold across the time axis. Plotting ρ over time reveals the time point of highest accuracy (maximum ρ). (B) Maximum ρ of 0.95 was observed at day -23 (range [-27, -13]; N = 53 patients). (C) Summary of concerted biological adaptations depicting a clock to labor. Angiogenic factors: Decreased Angiopoietin-2, sTie-2, and VEGF121. Aging fetal membranes: Increased PLXB2 and DDR1. Placental signaling: Increased Activin-A and Siglec-6. Coagulation capacity: Decreased ATIII and increased uPA. Immune responsiveness: Increased Cystatin C, increased pSTAT1 responses in NK and pDC upon IFN-α stimulation, and decreased granulocyte frequencies. A switch to prelabor biology occurs at day 23 (range [-27,13]; pink shaded phase) before the day of labor. The prelabor phase is characterized by immune regulation: Stagnating pSTAT1 responses in NK and pDC upon IFN-α stimulation, decreased basal IκB and pMK2 signals in CD4+ and CD8+ T cells, decreased pCREB in ncMC upon GM-CSF stimulation, decreased pSTAT6 responses in DC upon IFN-α stimulation, decreased pMK2 in B cells upon LPS stimulation, and decreased MyD88 responses in cMC upon LPS and GM-CSF stimulation. Regulation of Macrophage inhibitory cytokine-1 (MIC-1), Secretory Leukocyte Peptidase Inhibitor (SLPI), and Lymphocyte-activation gene 3 (LAG3). Surging Cystatin C and IL-1R4. Endocrine signaling: Surging 17-OHP isomers, 17-hydroxypregnenolone sulfate, and cortisol isomer.

FIG. 6. Analyses of the subcohort of patients with preterm (PT) labor. (A) Prediction accuracy (root mean square error (RMSE) in days) of models predicting the TL (original model trained on N = 48 term and N = 5 preterm birth; term-only model trained on N = 48 term birth). (B) Regression of predicted vs. true TL for original model plotting term birth data only (N= 48). (C) Regression of predicted vs. true TL for model trained in term birth data only (N = 48). (D) Regression of predicted vs. true TL for a model trained in term birth data only and tested in cohort of women with preterm birth (N = 5). (E-G) Comparison of feature-ranking by bootstrap analyses (original model and term-only model) shows significant correlations between ranks of most informative features, indicating that the original model captured biological parameters independent of studied gestational lengths. Related to FIG. 3.

FIG. 7. Metabolic features most informative for the integrated prediction model (N = 53 patients, n = 150 samples, training cohort). Features are ranked by model index. (A) C₂₁H₃₀O₃ 17-OHP isomer, (B) C₂₁H₃₀O₃ 17- OHP isomer, (C) C₂₁H₃₀O₃ 17-OHP isomer, (D) C₂₁H₃₀O₅ Cortisol isomer, (E) C₂₇H₄₂O₃, (F) 1- Methylhypoxanthine, (G) 17-OH pregnenolone sulfate, (H) 4-Aminohippuric acid, (I) Arabitol, Xylitol, (J) 5- Hydroxytryptophan, (K) N-Lactoylphenylalanine, (L) Pregnanolone sulfate. Lines represent linear/quadratic curves based on goodness-of-fit of a pattern fitting model (Akaike information criterion (AIC)); p-value associated with Fstatistic for comparison of fits (see table 5). See also table 4. Related to FIG. 4.

FIG. 8. Proteomic features most informative for the integrated prediction model (N = 53 patients, n = 150 samples, training cohort). Features are ranked by model index. (A) IL-1R4, (B) Plexin-B2 (PLXB2), (C) Discoidin domain receptor 1 (DDR1), (D) Angiopoietin-2, (E) VEGF121, (F) Cystatin C, (G) SLIT and NTRK-like protein 5 (SLTRK5), (H) Secr. Leukocyte Peptidase Inhibitor (SLPI), (I) Activin A, (J) Antithrombin III, (K) Macrophage inhibitory cytokine-1 (MIC-1), (L) Siglec-6, (M) uPA, (N) MMP12, (O) sTie-2, (P) LAG3, (Q) Endostatin, (R) GA733-1 protein. Lines represent linear/quadratic curves based on goodness-of-fit of a pattern fitting model (Akaike information criterion (AIC)); p-value associated with F-statistic for comparison of fits (see table 5). RFU = Relative Fluorescence Unit. See also table 4. Related to FIG. 4.

FIG. 9. Immune features most informative for the integrated prediction model (N = 53 patients, n = 150 samples, training cohort). Features are ranked by model index. (A) CD69-CD56dimCD16+NK, pSTAT1, IFNα, (B) Granulocytes (freq), (C) CD69+CD56dimCD16+NK, pSTAT1, IFNα, (D) CD62L+CD4Tnaive, pMAPKAPK2, IFNα, (E) ncMC, pCREB, GM-CSF, (F) CD69+CD8Tmem, pMK2, basal, (G) pDC, pSTAT1, IFNα, (H) B cells, pMK2, LPS, (I) CD4Tem, pMK2, basal, (J) CD69+CD8Tmem, pMK2, IFNα, (K) B cells (freq), (L) CCR5+CCR+CD4Tem, pNFκB, IL-2,4,6, (M) CCR+CCR2+CD4Tcm, IκB, basal, (N) DC, pSTAT6, IFNα, (O) DC, pMK2, basal. Lines represent linear/quadratic curves based on goodness-of-fit of a pattern fitting model (Akaike information criterion (AIC)); p-value associated with F-statistic for comparison of fits (see table 5). See also table 4. Related to FIG. 4.

FIG. 10. Innate immune responsiveness decelerates in the prelabor phase (N = 53 patients, n = 150 samples, training cohort). Classical monocytes (cMCs) in response to lipopolysaccharide (LPS) (A-C) and GM-CSF (D-F) show a decrease in MyD88-signaling responses (pP38 (A, D), pERK1/2 (B, E), and pCREB (C, F)) with approaching labor. Lines represent linear/quadratic curves based on goodness-of-fit of a pattern fitting model (Akaike information criterion (AIC)); p-value associated with F-statistic for comparison of fits. Related to FIG. 4.

FIG. 11. Basal adaptive immune activity in the prelabor phase (N = 53 patients, n = 150 samples, training cohort). Phosphorylation of STAT5 (pSTAT5) in naïve (A) and memory (B) CD4+ T cell subsets, naïve CD8+ T cells (C) and FoxP3+CD25+ regulatory CD4+ T cells (D), top informative features for the prediction of gestational age throughout pregnancy (Aghaeepour et al. 2017), increases with approaching labor, but is not informative for the prediction of time to labor (TL). Lines represent linear/quadratic curves based on goodness-of-fit of a pattern fitting model (Akaike information criterion (AIC)); p-value associated with F-statistic for comparison of fits. Related to FIG. 4.

FIG. 12. Gating strategy for mass cytometry analyses. Live, non-erythroid cell populations were used for analysis.

### DETAILED DESCRIPTION

These and other features of the present teachings will become more apparent from the description herein. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Most of the words used in this specification have the meaning that would be attributed to those words by one skilled in the art. Words specifically defined in the specification have the meaning provided in the context of the present teachings as a whole, and as are typically understood by those skilled in the art. In the event that a conflict arises between an art-understood definition of a word or phrase and a definition of the word or phrase as specifically taught in this specification, the specification shall control.

All publications, patents, and patent applications mentioned in this specification are herein incorporated by reference to the same extent as if each individual publication, patent, or patent application was specifically and individually indicated to be incorporated by reference.

It must be noted that, as used in the specification and the appended claims, the singular forms “a,” “an,” and “the” include plural referents unless the context clearly dictates otherwise.

Compositions and methods are provided for classification of patients during pregnancy according to their time to onset of labor, using a multi-omic analysis. Patterns of response are obtained by quantitating specific features, for a period of time during pregnancy, usually for at least two timepoints during pregnancy. The pattern of response is indicative of the patient’s time to onset of labor. Once a classification or prognosis has been made, it can be provided to a patient or caregiver. The classification can provide prognostic information to guide clinical decision making, both in terms of institution of and escalation of treatment, and in some cases may further include selection of a therapeutic agent or regimen.

The information obtained from the features can be used to (a) determine type and level of therapeutic intervention warranted and (b) to optimize the selection of therapeutic agents. With this approach, therapeutic regimens can be individualized and tailored according to the time to onset of labor, thereby providing a regimen that is individually appropriate.

The terms “subject,” “individual,” and “patient” are used interchangeably herein to refer to a vertebrate, preferably a mammal, more preferably a human. Mammalian species that provide samples for analysis include canines; felines; equines; bovines; ovines; etc. and primates, particularly humans. Animal models, particularly small mammals, e.g. murine, lagomorpha, etc. can be used for experimental investigations. The methods of the invention can be applied for veterinary purposes.

As used herein, the term “theranosis” refers to the use of results obtained from a diagnostic or prognostic method to direct the selection of, maintenance of, or changes to a therapeutic regimen, including but not limited to the choice of one or more therapeutic agents, changes in dose level, changes in dose schedule, changes in mode of administration, and changes in formulation. Diagnostic methods used to inform a theranosis can include any analysis that provides information on the state of a disease, condition, or symptom.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the amount of an agent that is sufficient to effect beneficial or desired results. The therapeutically effective amount will vary depending upon the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose will vary depending on the particular agent chosen, the dosing regimen to be followed, whether it is administered in combination with other compounds, timing of administration, the tissue to be imaged, and the physical delivery system in which it is carried.

“Suitable conditions” shall have a meaning dependent on the context in which this term is used. That is, when used in connection with an antibody, the term shall mean conditions that permit an antibody to bind to its corresponding antigen. When used in connection with contacting an agent to a cell, this term shall mean conditions that permit an agent capable of doing so to enter a cell and perform its intended function. In one embodiment, the term “suitable conditions” as used herein means physiological conditions.

The term “inflammatory” response is the development of a humoral (antibody mediated) and/or a cellular response, which cellular response may be mediated by antigen-specific T cells or their secretion products), and innate immune cells. An “immunogen” is capable of inducing an immunological response against itself on administration to a mammal or due to autoimmune disease.

The terms “biomarker,” “biomarkers,” “marker”, “features”, or “markers” for the purposes of the invention refer to, without limitation, metabolites, cells, e.g. immune cells, responsiveness of immune cells to stimulus, proteins together with their related metabolites, mutations, variants, polymorphisms, modifications, fragments, subunits, degradation products, elements, and other analytes or sample-derived measures. Markers can include expression levels of an intracellular protein or extracellular protein. Markers can also include combinations of any one or more of the foregoing measurements, including temporal trends and differences.

The term “features” is used herein to refer to such biomarkers, and may include one or more of: 331.2264_8.4 (17-OHP/P4 derivative); 331.2264_8.1 (17-OHP/P4 derivative); 331.2265_8.9 (17-OHP/P4 derivative); 361.2017_7.1 (Cortisol); 415.3204_12 (C₂₇H₄₂O₃); 151.0615_2.6 (1-Methylhypoxanthine); 411.1844_8.7 (17-OH pregnenolone sulfate); 193.0618_5.3 (4-Aminohippuric acid); 151.0612_6 (Arabitol, Xylitol); 219.0774_6.3 (5-Hydroxytryptophan); 236.0929_4.3 (N-Lactoylphenylalanine); 397.205_10.6 (6 (Pregnanolone sulfate); IL-1R4; Plexin-B2 (PLXB2); Discoidin domain receptor 1 (DDR1); Angiopoietin-2; Vascular Endothelial Growth Factor 121; Cystatin C; SLIT and NTRK-like protein 5 (SLTRK5); Secr. Leukocyte Peptidase Inhibitor (SLPI); Activin A; Antithrombin III; Macrophage inhibitory cytokine-1 (MIC-1); Siglec-6; urokinase-type Plasminogen Activator (uPA); Matrix Metalloproteinase (MMP) 12; Soluble tunica interna endothelial cell kinase (sTie)-2; LAG3; Endostatin; GA733-1 protein; CD69⁻CD56^{lo}CD16⁺NK, pSTAT1, IFNα; Granulocytes (freq); CD69⁺CD56^{lo}CD16⁺NK, pSTAT1, IFNα; CD62L⁺CD4Tₙₐᵢᵥₑ, pMAPKAPK2, IFNα; ncMC, pCREB, GM-CSF; CD69⁺CD8Tₘₑₘ, pMAPKAPK2, basal; pDC, pSTAT1, IFNα; B cells, pMAPKAPK2, LPS; CD4Tₑₘ, pMAPKAPK2, basal; CD69⁺CD8Tₘₑₘ, pMAPKAPK2, IFNα; B cells (freq); CCR5⁺CCR⁺CD4Tₑₘ, pNFκB, IL-2,4,6; CCR⁺CCR2⁺CD4T_{cm}, IκB, basal; DC, pSTAT6, IFNα; DC, pMAPKAPK2, basal. Immune cells may be notated with an activating agent and measurable intracellular protein. For example, the feature “DC, pSTAT6, IFNα” refers to changes in pSTAT6 in dendritic cells, in response to IFNα. The feature “CD69⁺CD8Tₘₑₘ, pMAPKAPK2, IFNα” refers to CD8+ T memory cells changes in pMAPKAPK2 response to IFNα.

In some embodiments the set of features being analyzed comprises or consists of: IL-1 receptor type 4 (IL-1R4); Activin-A; Sialic Acid Binding Ig Like Lectin (Siglec)-6; antithrombin III (ATIII); soluble tunica interna endothelial cell kinase (sTie)-2; PLXB2; DDR1; Angiopoietin-2; and vascular endothelial growth factor (VEGF)121.

In some embodiments the set of features being analyzed comprises or consists of: cortisol, Angiopoietin-2; granulocytes (frequency); isomers of 17-hydroxyprogesterone (17-OHP); 17-hydroxypregnenolone sulfate; IL-1 receptor type 4 (IL-1R4); dendritic cells pSTAT6 response to interferon α; soluble tunica interna endothelial cell kinase (sTie)-2; and CD69⁻CD56^{lo}CD16⁺NK cell pSTAT1 response to IFNα.

In some embodiments, a set of features comprises: isomers of 17-hydroxyprogesterone (17-OHP); and 17-hydroxypregnenolone sulfate.

Features are typically measured at two or more time points, and may be measured at 3, 4, 5 or more time points. Time points may be monthly, biweekly, weekly, every 2, 3, 4,, 5, 6 days, etc. The trajectory of change is disclosed herein, e.g. as shown in Tables 4 and 5. Classifying the dynamic behavior of each feature revealed three general trajectory patterns on the basis of the goodness of fit of a pattern-fitting model: linear progression model: linear progression (degree 1) or quadratic progression, including accelerating (surging of an increasing or decreasing pattern over time) (degree 2a) or decelerating (plateauing of an increasing or decreasing pattern over time) (degree 2b) progression. Metabolomic and proteomic model features were predominantly classified as degree 1 (constant rate). In contrast, immune cell trajectories predominantly followed a degree 2b (decelerating) pattern.

The presence of degree 2 (quadratic) trajectories across all omic datasets points toward a period of disruption with approaching labor that resonated across all measured biological systems. Identifying the timing of such a nonlinear transition is clinically relevant because it defines when the assessment of peripheral blood analytes is linked to prelabor biology rather than a reflection of the biology relevant for the maintenance of pregnancy. A piecewise fused LASSO regression analysis was used to provide an estimate as to when before the day of labor such a transition occurs. This approach combined the predictions rho (ρ) of two LASSO regression models built using the data points before or after a given TL threshold, while varying the threshold across all time points. A maximum ρ value was reached when the models on each side of the threshold contained distinct yet top informative biological features that, when combined, reached maximal predictive accuracy. The piecewise fused LASSO regression analysis produced a maximum at 23 days before the day of labor (range [-27, -13] days; ρ at -23 days = 0.95).

An initial time point may be in the second or third trimester of pregnancy, usually the time points are within the predicted last 100 days of pregnancy. In some embodiments an initial time point for analysis is around about 100 days prior to initially predicted labor, and subsequent time points include analysis within the last 2-6 weeks of initially predicted length of pregnancy. The time points will desirably encompass the timing of the non-linear trajectory transition, which may be around 2 to 4 weeks prior to actual day of delivery. For example, blood samples may be taken every two weeks of the final trimester of pregnancy.

To “analyze” includes determining a set of values associated with a sample by measurement of a marker (such as, e.g., presence or absence of a marker or constituent expression levels) in the sample and comparing the measurement against measurement in a sample or set of samples from the same subject or other control subject(s). The markers of the present teachings can be analyzed by any of various conventional methods known in the art. To “analyze” can include performing a statistical analysis, e.g. normalization of data, determination of statistical significance, determination of statistical correlations, clustering algorithms, and the like.

A “sample” in the context of the present teachings refers to any biological sample that is isolated from a subject, generally a blood sample, which may comprise circulating immune cells. Proteomic and metabolomic features can be analyzed with blood derivatives, e.g. plasma, serum, etc. A sample can include, without limitation, an aliquot of body fluid, plasma, whole blood, PBMC (white blood cells or leucocytes), tissue biopsies, synovial fluid, lymphatic fluid, ascites fluid, and interstitial or extracellular fluid. “Blood sample” can refer to whole blood or a fraction thereof, including blood cells, plasma, white blood cells or leucocytes. Samples can be obtained from a subject by means including but not limited to venipuncture, biopsy, needle aspirate, lavage, scraping, surgical incision, or intervention or other means known in the art.

Optionally samples are activated ex vivo, which as used herein, refers to the contacting of a sample, e.g. a blood sample or cells derived therefrom, outside of the body with a stimulating agent. In some embodiments whole blood is preferred. The sample may be diluted or suspended in a suitable medium that maintains the viability of the cells, e.g. minimal media, PBS, etc. The sample can be fresh or frozen. Stimulating agents of interest include those agents that activate innate or adaptive cells, e.g. and without limitation, LPS (1 µg/mL) and/or IFN-α (100 ng/mL). Generally the activation of cells ex vivo is compared to a negative control, e.g. medium only, or an agent that does not elicit activation. The cells are incubated for a period of time sufficient for activation. For example, the time for action can be up to about 1 hour, up to about 45 minutes, up to about 30 minutes, up to about 15 minutes, and may be up to about 10 minutes or up to about 5 minutes. In some embodiments the period of time is up to about 24 hours. Following activation, the cells are fixed for analysis.

A “dataset” is a set of numerical values resulting from evaluation of a sample (or population of samples) under a desired condition. The values of the dataset can be obtained, for example, by experimentally obtaining measures from a sample and constructing a dataset from these measurements; or alternatively, by obtaining a dataset from a service provider such as a laboratory, or from a database or a server on which the dataset has been stored. Similarly, the term “obtaining a dataset associated with a sample” encompasses obtaining a set of data determined from at least one sample. Obtaining a dataset encompasses obtaining a sample, and processing the sample to experimentally determine the data, e.g., via measuring antibody binding, or other methods of quantitating a signaling response. The phrase also encompasses receiving a set of data, e.g., from a third party that has processed the sample to experimentally determine the dataset.

“Measuring” or “measurement” in the context of the present teachings refers to determining the presence, absence, quantity, amount, or effective amount of a substance in a clinical or subject-derived sample, including the presence, absence, or concentration levels of such substances, and/or evaluating the values or categorization of a subject’s clinical parameters based on a control, e.g. baseline levels of the marker.

Classification can be made according to predictive modeling methods that set a threshold for determining the probability that a sample belongs to a given class. The probability preferably is at least 50%, or at least 60% or at least 70% or at least 80% or higher. Classifications also can be made by determining whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

The predictive ability of a model can be evaluated according to its ability to provide a quality metric, e.g. AUC or accuracy, of a particular value, or range of values. In some embodiments, a desired quality threshold is a predictive model that will classify a sample with an accuracy of at least about 0.7, at least about 0.75, at least about 0.8, at least about 0.85, at least about 0.9, at least about 0.95, or higher. As an alternative measure, a desired quality threshold can refer to a predictive model that will classify a sample with an AUC (area under the curve) of at least about 0.7, at least about 0.75, at least about 0.8, at least about 0.85, at least about 0.9, or higher.

As is known in the art, the relative sensitivity and specificity of a predictive model can be “tuned” to favor either the selectivity metric or the sensitivity metric, where the two metrics have an inverse relationship. The limits in a model as described above can be adjusted to provide a selected sensitivity or specificity level, depending on the particular requirements of the test being performed. One or both of sensitivity and specificity can be at least about at least about 0.7, at least about 0.75, at least about 0.8, at least about 0.85, at least about 0.9, or higher.

“Affinity reagent”, or “specific binding member” may be used to refer to an affinity reagent, such as an antibody, ligand, etc. that selectively binds to a protein or marker of the invention. The term “affinity reagent” includes any molecule, e.g., peptide, nucleic acid, small organic molecule. For some purposes, an affinity reagent selectively binds to a cell surface marker, e.g. CD3, CD14, CD66, HLA-DR, CD11b, CD33, CD45, CD235, CD61, CD19, CD4, CD8, CD123, CCR7, and the like. For other purposes an affinity reagent selectively binds to a cellular signaling protein, particularly one which is capable of detecting an activation state of a signaling protein over another activation state of the signaling protein. Signaling proteins of interest include, without limitation, pSTAT3, pSTAT1, pCREB, pSTAT6, pPLCγ2, pSTAT5, pSTAT4, pERK, pP38, prpS6, pNF-κB (p65), pMAPKAPK2, pP90RSK, etc.

Other affinity reagents of interest bind to plasma proteins, e.g. Endostatin, Angiopoietin-2, Cystatin C, GA733-1-protein, Siglec 6, Activin A, Antithrombin III, sTie 2, DDR1, uPA, IL-1R4, MIC1, SLPI, MMP12, SLIK5, VEGF121, LAG3, PLXB2.

Metabolites of interest for detection include 151.0612_6 (Arabitol, Xylitol), 151.0615_2.6 (1-Methylhypoxanthine), 193.0618_5.3 (4-Aminohyppuric acid), 219.0774_6.3 (5-Hydroxytryptophan), 236.0929_4.3 (N-Lactoylphenylalanine), 331.2264_8.1 (17-Hydroxyprogesterone), 331.2264_8.4 (17-Hydroxyprogesterone), 331.2265_8.9 (17-Hydroxyprogesterone), 361.2017_7.1 (Cortisol), 397.205_10.6 (Pregnanolone sulfate), 411.1844_8.7 (17-Hydroxypregnenolone sulfate), 415.3204_12 (C27H42O3).

In some embodiments, the affinity reagent is a peptide, polypeptide, oligopeptide or a protein, particularly antibodies and specific binding fragments and variants thereof. The peptide, polypeptide, oligopeptide or protein can be made up of naturally occurring amino acids and peptide bonds, or synthetic peptidomimetic structures. Thus “amino acid”, or “peptide residue”, as used herein include both naturally occurring and synthetic amino acids. Proteins including non-naturally occurring amino acids can be synthesized or in some cases, made recombinantly; see van Hest et al., FEBS Lett 428:(I-2) 68-70 May 22, 1998 and Tang et al., Abstr. Pap Am. Chem. S218: U138 Part 2 Aug. 22, 1999, both of which are expressly incorporated by reference herein.

The term “antibody” includes full length antibodies and antibody fragments, and can refer to a natural antibody from any organism, an engineered antibody, or an antibody generated recombinantly for experimental, therapeutic, or other purposes as further defined below. Examples of antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies. The term “antibody” comprises monoclonal and polyclonal antibodies. Antibodies can be antagonists, agonists, neutralizing, inhibitory, or stimulatory. They can be humanized, glycosylated, bound to solid supports, and possess other variations.

Many antibodies, many of which are commercially available (for example, see Cell Signaling Technology, www.cellsignal.com or Becton Dickinson, www.bd.com) have been produced which specifically bind to the phosphorylated isoform of a protein but do not specifically bind to a non-phosphorylated isoform of a protein. Many such antibodies have been produced for the study of signal transducing proteins which are reversibly phosphorylated. Particularly, many such antibodies have been produced which specifically bind to phosphorylated, activated isoforms of protein and plasma proteins. Examples of proteins that can be analyzed with the methods described herein include, but are not limited to, NF-κB, CREB, STAT5, STAT1, STAT3, etc.

The methods the invention may utilize affinity reagents comprising a label, labeling element, or tag. By label or labeling element is meant a molecule that can be directly (i.e., a primary label) or indirectly (i.e., a secondary label) detected; for example a label can be visualized and/or measured or otherwise identified so that its presence or absence can be known.

A compound can be directly or indirectly conjugated to a label which provides a detectable signal, e.g. non-radioactive isotopes, radioisotopes, fluorophores, enzymes, antibodies, particles such as magnetic particles, chemiluminescent molecules, molecules that can be detected by mass spec, or specific binding molecules, etc. Specific binding molecules include pairs, such as biotin and streptavidin, digoxin and anti-digoxin etc. Examples of labels include, but are not limited to, metal isotopes, optical fluorescent and chromogenic dyes including labels, label enzymes and radioisotopes. In some embodiments of the invention, these labels can be conjugated to the affinity reagents. In some embodiments, one or more affinity reagents are uniquely labeled.

Labels include optical labels such as fluorescent dyes or moieties. Fluorophores can be either “small molecule” fluors, or proteinaceous fluors (e.g. green fluorescent proteins and all variants thereof). In some embodiments, activation state-specific antibodies are labeled with quantum dots as disclosed by Chattopadhyay et al. (2006) Nat. Med. 12, 972-977. Quantum dot labeled antibodies can be used alone or they can be employed in conjunction with organic fluorochrome- conjugated antibodies to increase the total number of labels available. As the number of labeled antibodies increase so does the ability for subtyping known cell populations.

Antibodies can be labeled using chelated or caged lanthanides as disclosed by Erkki et al.(1988) J. Histochemistry Cytochemistry, 36:1449-1451, and U.S. Pat. No. 7,018850. Other labels are tags suitable for Inductively Coupled Plasma Mass Spectrometer (ICP-MS) as disclosed in Tanner et al. (2007) Spectrochimica Acta Part B: Atomic Spectroscopy 62(3):188-195. Isotope labels suitable for mass cytometry may be used, for example as described in published application US 2012-0178183.

Alternatively, detection systems based on FRET can be used. FRET find use in the invention, for example, in detecting activation states that involve clustering or multimerization wherein the proximity of two FRET labels is altered due to activation. In some embodiments, at least two fluorescent labels are used which are members of a fluorescence resonance energy transfer (FRET) pair.

When using fluorescent labeled components in the methods and compositions of the present invention, it will be recognized that different types of fluorescent monitoring systems, e.g., cytometric measurement device systems, can be used to practice the invention. In some embodiments, flow cytometric systems are used, or systems dedicated to high throughput screening, e.g. 96 well or greater microtiter plates. Methods of performing assays on fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego:Academic Press (1989), pp. 219-243; Turro, N. J., Modern Molecular Photochemistry, Menlo Park: Benjamin/Cummings Publishing Col, Inc. (1978), pp. 296-361.

The detecting, sorting, or isolating step of the methods of the present invention can entail fluorescence-activated cell sorting (FACS) techniques, where FACS is used to select cells from the population containing a particular surface marker, or the selection step can entail the use of magnetically responsive particles as retrievable supports for target cell capture and/or background removal. A variety of FACS systems are known in the art and can be used in the methods of the invention (see e.g., W099/54494, filed Apr. 16, 1999; U.S. Ser. No. 20010006787, filed Jul. 5, 2001, each expressly incorporated herein by reference).

In some embodiments, a FACS cell sorter (e.g. a FACSVantage™ Cell Sorter, Becton Dickinson Immunocytometry Systems, San Jose, Calif.) is used to sort and collect cells based on their activation profile (positive cells) in the presence or absence of an increase in activation level in an signaling protein in response to a modulator. Other flow cytometers that are commercially available include the LSR II and the Canto II both available from Becton Dickinson. See Shapiro, Howard M., Practical Flow Cytometry, 4th Ed., John Wiley & Sons, Inc., 2003 for additional information on flow cytometers.

In some embodiments, the cells are first contacted with labeled activation state-specific affinity reagents (e.g. antibodies) directed against specific activation state of specific signaling proteins. In such an embodiment, the amount of bound affinity reagent on each cell can be measured by passing droplets containing the cells through the cell sorter. By imparting an electromagnetic charge to droplets containing the positive cells, the cells can be separated from other cells. The positively selected cells can then be harvested in sterile collection vessels. These cell-sorting procedures are described in detail, for example, in the FACSVantage™ . Training Manual, with particular reference to sections 3-11 to 3-28 and 10-1 to 10-17, which is hereby incorporated by reference in its entirety. See the patents, applications and articles referred to, and incorporated above for detection systems.

In some embodiments, the activation level of an signaling protein is measured using Inductively Coupled Plasma Mass Spectrometer (ICP-MS). An affinity reagent that has been labeled with a specific element binds to a marker of interest. When the cell is introduced into the ICP, it is atomized and ionized. The elemental composition of the cell, including the labeled affinity reagent that is bound to the signaling protein, is measured. The presence and intensity of the signals corresponding to the labels on the affinity reagent indicates the level of the signaling protein on that cell (Tanner et al. Spectrochimica Acta Part B: Atomic Spectroscopy, 2007 Mar;62(3):188-195.).

Mass cytometry, e.g. as described in the Examples provided herein, finds use on analysis. Mass cytometry, or CyTOF (DVS Sciences), is a variation of flow cytometry in which antibodies are labeled with heavy metal ion tags rather than fluorochromes. Readout is by time-of-flight mass spectrometry. This allows for the combination of many more antibody specificities in a single samples, without significant spillover between channels. For example, see Bodenmiller at a. (2012) Nature Biotechnology 30:858-867.

The present invention incorporates information disclosed in other applications and texts. The following patent and other publications are hereby incorporated by reference in their entireties: Alberts et al., The Molecular Biology of the Cell, 4th Ed., Garland Science, 2002; Vogelstein and Kinzler, The Genetic Basis of Human Cancer, 2d Ed., McGraw Hill, 2002; Michael, Biochemical Pathways, John Wiley and Sons, 1999; Weinberg, The Biology of Cancer, 2007; Immunobiology, Janeway et al. 7th Ed., Garland, and Leroith and Bondy, Growth Factors and Cytokines in Health and Disease, A Multi Volume Treatise, Volumes 1A and IB, Growth Factors, 1996.

Unless otherwise apparent from the context, all elements, steps or features of the invention can be used in any combination with other elements, steps or features.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. Due to biological functional equivalency considerations, changes can be made in protein structure without affecting the biological action in kind or amount. All such modifications are intended to be included within the scope of the appended claims.

The subject methods are used for prophylactic or therapeutic purposes. As used herein, the term “treating” is used to refer to both prevention of relapses, and treatment of pre-existing conditions. For example, the prevention of inflammatory disease can be accomplished by administration of the agent prior to development of a relapse. The treatment of ongoing disease, where the treatment stabilizes or improves the clinical symptoms of the patient, is of particular interest.

### Methods of the Invention

Multi-omic analysis of biological samples, e.g. blood-based samples, obtained from an individual during pregnancy is used to obtain a determination of changes in immune cell subsets, in plasma proteins and in metabolites. It is surprisingly found that the interactome of these features is predictive of the time to onset of labor.

The sample can be any suitable type that allows for the analysis of one or more cells, proteins and metabolites, preferably a blood sample. Samples can be obtained once or multiple times from an individual. Multiple samples can be obtained from different locations in the individual, at different times from the individual, or any combination thereof.

When samples are obtained as a series, e.g., a series of blood samples obtained during pregnancy, the samples can be obtained at fixed intervals, at intervals determined by the status of the most recent sample or samples or by other characteristics of the individual, or some combination thereof. It will be appreciated that an interval may not be exact, according to an individual’s availability for sampling and the availability of sampling facilities, thus approximate intervals corresponding to an intended interval scheme are encompassed by the invention. Generally, the most easily obtained samples are fluid samples. In some embodiments the sample or samples is blood.

One or more cells or cell types, proteins and metabolites can be isolated from body samples. The cells can be separated from body samples by red cell lysis, centrifugation, elutriation, density gradient separation, apheresis, affinity selection, panning, FACS, centrifugation with Hypaque, solid supports (magnetic beads, beads in columns, or other surfaces) with attached antibodies, etc. By using antibodies specific for markers identified with particular cell types, a relatively homogeneous population of cells can be obtained. Alternatively, a heterogeneous cell population can be used, e.g. circulating peripheral blood mononuclear cells.

In some embodiments, a phenotypic profile of a population of cells is determined by measuring the activation level of a signaling protein. The methods and compositions of the invention can be employed to examine and profile the status of any signaling protein in a cellular pathway, or collections of such signaling proteins. Single or multiple distinct pathways can be profiled (sequentially or simultaneously), or subsets of signaling proteins within a single pathway or across multiple pathways can be examined (sequentially or simultaneously).

In some embodiments, the basis for classifying cells is that the distribution of activation levels for one or more specific signaling proteins will differ among different phenotypes. A certain activation level, or more typically a range of activation levels for one or more signaling proteins seen in a cell or a population of cells, is indicative that that cell or population of cells belongs to a distinctive phenotype. Other measurements, such as cellular levels (e.g., expression levels) of biomolecules that may not contain signaling proteins, can also be used to classify cells in addition to activation levels of signaling proteins; it will be appreciated that these levels also will follow a distribution. Thus, the activation level or levels of one or more signaling proteins, optionally in conjunction with the level of one or more biomolecules that may or may not contain signaling proteins, of a cell or a population of cells can be used to classify a cell or a population of cells into a class. It is understood that activation levels can exist as a distribution and that an activation level of a particular element used to classify a cell can be a particular point on the distribution but more typically can be a portion of the distribution. In addition to activation levels of intracellular signaling proteins, levels of intracellular or extracellular biomolecules, e.g., proteins, can be used alone or in combination with activation states of signaling proteins to classify cells. Further, additional cellular elements, e.g., biomolecules or molecular complexes such as RNA, DNA, carbohydrates, metabolites, and the like, can be used in conjunction with activation states or expression levels in the classification of cells encompassed here.

In some embodiments of the invention, different gating strategies can be used in order to analyze a specific cell population (e.g., only CD4⁺ T cells) in a sample of mixed cell population. These gating strategies can be based on the presence of one or more specific surface markers. The following gate can differentiate between dead cells and live cells and the subsequent gating of live cells classifies them into, e.g. myeloid blasts, monocytes and lymphocytes. A clear comparison can be carried out by using two-dimensional contour plot representations, two-dimensional dot plot representations, and/or histograms.

The immune cells are analyzed for the presence of an activated form of a signaling protein of interest. Signaling proteins of interest include, without limitation, pSTAT3, pSTAT1, pCREB, pSTAT6, pPLCγ2, pSTAT5, pSTAT4, pERK, pP38, prpS6, pNF-κB (p65), pMAPKAPK2, and pP90RSK. pSTAT1 and pSTAT5 are of particular interest. To determine if a change is significant the signal in a patient’s baseline sample can be compared to a reference scale from a cohort of patients with known outcomes.

Samples may be obtained at one or more time points. Where a sample at a single time point is used, comparison is made to a reference “base line” level for the feature, which may be obtained from a normal control, a pre-determined level obtained from one or a population of individuals, from a negative control for ex vivo activation, and the like.

In some embodiment, the methods of the invention include the use of liquid handling components. The liquid handling systems can include robotic systems comprising any number of components. In addition, any or all of the steps outlined herein can be automated; thus, for example, the systems can be completely or partially automated. See USSN 61/048,657. As will be appreciated by those in the art, there are a wide variety of components which can be used, including, but not limited to, one or more robotic arms; plate handlers for the positioning of microplates; automated lid or cap handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination- free liquid, particle, cell, and organism transfers. This instrument performs automated replication of microplate samples to filters, membranes, and/or daughter plates, high-density transfers, full-plate serial dilutions, and high capacity operation.

In some embodiments, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station. In some embodiments, the methods of the invention include the use of a plate reader.

In some embodiments, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some embodiments, the instrumentation will include a detector, which can be a wide variety of different detectors, depending on the labels and assay. In some embodiments, useful detectors include a microscope(s) with multiple channels of fluorescence; plate readers to provide fluorescent, ultraviolet and visible spectrophotometric detection with single and dual wavelength endpoint and kinetics capability, fluorescence resonance energy transfer (FRET), luminescence, quenching, two-photon excitation, and intensity redistribution; CCD cameras to capture and transform data and images into quantifiable formats; and a computer workstation.

In some embodiments, the robotic apparatus includes a central processing unit which communicates with a memory and a set of input/output devices (e.g., keyboard, mouse, monitor, printer, etc.) through a bus. Again, as outlined below, this can be in addition to or in place of the CPU for the multiplexing devices of the invention. The general interaction between a central processing unit, a memory, input/output devices, and a bus is known in the art. Thus, a variety of different procedures, depending on the experiments to be run, are stored in the CPU memory.

The differential presence of these markers is shown to provide for prognostic evaluations to detect individuals having a time to onset of labor. In general, such prognostic methods involve determining the presence or level of activated signaling proteins in an individual sample of immune cells. Detection can utilize one or a panel of specific binding members, e.g. a panel or cocktail of binding members specific for one, two, three, four, five or more markers.

### Data Analysis

A signature pattern can be generated from a biological sample using any convenient protocol, for example as described below. The readout can be a mean, average, median or the variance or other statistically or mathematically-derived value associated with the measurement. The marker readout information can be further refined by direct comparison with the corresponding reference or control pattern. A binding pattern can be evaluated on a number of points: to determine if there is a statistically significant change at any point in the data matrix relative to a reference value; whether the change is an increase or decrease in the binding; whether the change is specific for one or more physiological states, and the like. The absolute values obtained for each marker under identical conditions will display a variability that is inherent in live biological systems and also reflects the variability inherent between individuals.

Following obtainment of the signature pattern from the sample being assayed, the signature pattern can be compared with a reference or base line profile to make a prognosis regarding the phenotype of the patient from which the sample was obtained/derived. Additionally, a reference or control signature pattern can be a signature pattern that is obtained from a sample of a patient known to have a normal pregnancy.

In certain embodiments, the obtained signature pattern is compared to a single reference/control profile to obtain information regarding the phenotype of the patient being assayed. In yet other embodiments, the obtained signature pattern is compared to two or more different reference/control profiles to obtain more in depth information regarding the phenotype of the patient. For example, the obtained signature pattern can be compared to a positive and negative reference profile to obtain confirmed information regarding whether the patient has the phenotype of interest.

Samples can be obtained from the tissues or fluids of an individual. For example, samples can be obtained from whole blood, tissue biopsy, serum, etc. Other sources of samples are body fluids such as lymph, cerebrospinal fluid, and the like. Also included in the term are derivatives and fractions of such cells and fluids

In order to identify profiles that are indicative of responsiveness, a statistical test can provide a confidence level for a change in the level of markers between the test and reference profiles to be considered significant. The raw data can be initially analyzed by measuring the values for each marker, usually in duplicate, triplicate, quadruplicate or in 5-10 replicate features per marker. A test dataset is considered to be different than a reference dataset if one or more of the parameter values of the profile exceeds the limits that correspond to a predefined level of significance.

To provide significance ordering, the false discovery rate (FDR) can be determined. First, a set of null distributions of dissimilarity values is generated. In one embodiment, the values of observed profiles are permuted to create a sequence of distributions of correlation coefficients obtained out of chance, thereby creating an appropriate set of null distributions of correlation coefficients (see Tusher et al. (2001) PNAS 98, 5116-21, herein incorporated by reference). This analysis algorithm is currently available as a software “plug-in” for Microsoft Excel know as Significance Analysis of Microarrays (SAM). The set of null distribution is obtained by: permuting the values of each profile for all available profiles; calculating the pair-wise correlation coefficients for all profile; calculating the probability density function of the correlation coefficients for this permutation; and repeating the procedure for N times, where N is a large number, usually 300. Using the N distributions, one calculates an appropriate measure (mean, median, etc.) of the count of correlation coefficient values that their values exceed the value (of similarity) that is obtained from the distribution of experimentally observed similarity values at given significance level.

The FDR is the ratio of the number of the expected falsely significant correlations (estimated from the correlations greater than this selected Pearson correlation in the set of randomized data) to the number of correlations greater than this selected Pearson correlation in the empirical data (significant correlations). This cut-off correlation value can be applied to the correlations between experimental profiles.

For SAM, Z-scores represent another measure of variance in a dataset, and are equal to a value of X minus the mean of X, divided by the standard deviation. A Z-Score tells how a single data point compares to the normal data distribution. A Z-score demonstrates not only whether a datapoint lies above or below average, but how unusual the measurement is. The standard deviation is the average distance between each value in the dataset and the mean of the values in the dataset.

Using the aforementioned distribution, a level of confidence is chosen for significance. This is used to determine the lowest value of the correlation coefficient that exceeds the result that would have obtained by chance. Using this method, one obtains thresholds for positive correlation, negative correlation or both. Using this threshold(s), the user can filter the observed values of the pairwise correlation coefficients and eliminate those that do not exceed the threshold(s). Furthermore, an estimate of the false positive rate can be obtained for a given threshold. For each of the individual “random correlation” distributions, one can find how many observations fall outside the threshold range. This procedure provides a sequence of counts. The mean and the standard deviation of the sequence provide the average number of potential false positives and its standard deviation. Alternatively, any convenient method of statistical validation can be used.

The data can be subjected to non-supervised hierarchical clustering to reveal relationships among profiles. For example, hierarchical clustering can be performed, where the Pearson correlation is employed as the clustering metric. One approach is to consider a patient disease dataset as a “learning sample” in a problem of “supervised learning”. CART is a standard in applications to medicine (Singer (1999) Recursive Partitioning in the Health Sciences, Springer), which can be modified by transforming any qualitative features to quantitative features; sorting them by attained significance levels, evaluated by sample reuse methods for Hotelling’s T² statistic; and suitable application of the lasso method. Problems in prediction are turned into problems in regression without losing sight of prediction, indeed by making suitable use of the Gini criterion for classification in evaluating the quality of regressions.

Other methods of analysis that can be used include logistic regression. One method of logic regression Ruczinski (2003) Journal of Computational and Graphical Statistics 12:475-512. Logic regression resembles CART in that its classifier can be displayed as a binary tree. It is different in that each node has Boolean statements about features that are more general than the simple “and” statements produced by CART.

Another approach is that of nearest shrunken centroids (Tibshirani (2002) PNAS 99:6567-72). The technology is k-means-like, but has the advantage that by shrinking cluster centers, one automatically selects features (as in the lasso) so as to focus attention on small numbers of those that are informative. The approach is available as Prediction Analysis of Microarrays (PAM) software, a software “plug-in” for Microsoft Excel, and is widely used. Two further sets of algorithms are random forests (Breiman (2001) Machine Learning 45:5-32 and MART (Hastie (2001) The Elements of Statistical Learning, Springer). These two methods are already “committee methods.” Thus, they involve predictors that “vote” on outcome. Several of these methods are based on the “R” software, developed at Stanford University, which provides a statistical framework that is continuously being improved and updated in an ongoing basis.

Other statistical analysis approaches including principle components analysis, recursive partitioning, predictive algorithms, Bayesian networks, and neural networks.

These tools and methods can be applied to several classification problems. For example, methods can be developed from the following comparisons: i) all cases versus all controls, ii) all cases versus nonresponsive controls, iii) all cases versus responsive controls.

In a second analytical approach, variables chosen in the cross-sectional analysis are separately employed as predictors. Given the specific outcome, the random lengths of time each patient will be observed, and selection of proteomic and other features, a parametric approach to analyzing responsiveness can be better than the widely applied semi-parametric Cox model. A Weibull parametric fit of survival permits the hazard rate to be monotonically increasing, decreasing, or constant, and also has a proportional hazards representation (as does the Cox model) and an accelerated failure-time representation. All the standard tools available in obtaining approximate maximum likelihood estimators of regression coefficients and functions of them are available with this model.

In addition the Cox models can be used, especially since reductions of numbers of covariates to manageable size with the lasso will significantly simplify the analysis, allowing the possibility of an entirely nonparametric approach to survival.

The analysis and database storage can be implemented in hardware or software, or a combination of both. In one embodiment of the invention, a machine-readable storage medium is provided, the medium comprising a data storage material encoded with machine readable data which, when using a machine programmed with instructions for using said data, is capable of displaying a any of the datasets and data comparisons of this invention. Such data can be used for a variety of purposes, such as patient monitoring, initial diagnosis, and the like. Preferably, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer can be, for example, a personal computer, microcomputer, or workstation of conventional design.

Each program is preferably implemented in a high level procedural or object oriented programming language to communicate with a computer system. However, the programs can be implemented in assembly or machine language, if desired. In any case, the language can be a compiled or interpreted language. Each such computer program is preferably stored on a storage media or device readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

A variety of structural formats for the input and output means can be used to input and output the information in the computer-based systems of the present invention. One format for an output means test datasets possessing varying degrees of similarity to a trusted profile. Such presentation provides a skilled artisan with a ranking of similarities and identifies the degree of similarity contained in the test pattern.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

### Kits

In some embodiments, the invention provides kits for the classification, diagnosis, prognosis, theranosis, and/or prediction of an outcome during pregnancy in a subject. The kit may further comprise a software package for data analysis of the cellular state and its physiological status, which may include reference profiles for comparison with the test profile and comparisons to other analyses as referred to above. The kit may also include instructions for use for any of the above applications.

Kits provided by the invention may comprise one or more of the affinity reagents described herein. A kit may also include other reagents that are useful in the invention, such as modulators, fixatives, containers, plates, buffers, therapeutic agents, instructions, and the like.

Kits provided by the invention can comprise one or more labeling elements. Non-limiting examples of labeling elements include small molecule fluorophores, proteinaceous fluorophores, radioisotopes, enzymes, antibodies, chemiluminescent molecules, biotin, streptavidin, digoxigenin, chromogenic dyes, luminescent dyes, phosphorous dyes, luciferase, magnetic particles, beta-galactosidase, amino groups, carboxy groups, maleimide groups, oxo groups and thiol groups, quantum dots, chelated or caged lanthanides, isotope tags, radiodense tags, electron- dense tags, radioactive isotopes, paramagnetic particles, agarose particles, mass tags, e-tags, nanoparticles, and vesicle tags.

In some embodiments, the kits of the invention enable the detection of proteins by sensitive cellular assay methods, such as ELISA, IHC and flow cytometry, which are suitable for the clinical detection, classification, diagnosis, prognosis, theranosis, and outcome prediction.

Such kits may additionally comprise one or more therapeutic agents. The kit may further comprise a software package for data analysis of the physiological status, which may include reference profiles for comparison with the test profile.

Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

### Reports

In some embodiments, providing an evaluation of a subject for a classification, diagnosis, prognosis, theranosis, and/or prediction of an outcome during pregnancy includes generating a written report that includes the artisan’s assessment of the subject’s state of health, including, for example, a “diagnosis assessment”, of the subject’s prognosis, i.e. a “prognosis assessment”, and/or of possible treatment regimens, i.e. a “treatment assessment”. Thus, a subject method may further include a step of generating or outputting a report providing the results of an assessment, which report can be provided in the form of an electronic medium (e.g., an electronic display on a computer monitor), or in the form of a tangible medium (e.g., a report printed on paper or other tangible medium).

A “report,” as described herein, is an electronic or tangible document which includes report elements that provide information of interest relating to a diagnosis assessment, a prognosis assessment, and/or a treatment assessment and its results. A subject report can be completely or partially electronically generated. A subject report includes at least a diagnosis assessment, i.e. a diagnosis as to whether a subject will have a particular clinical response during pregnancy, and/or a suggested course of treatment to be followed. A subject report can further include one or more of: 1) information regarding the testing facility; 2) service provider information; 3) subject data; 4) sample data; 5) an assessment report, which can include various information including: a) test data, where test data can include an analysis of cellular signaling responses to activation, b) reference values employed, if any.

The report may include information about the testing facility, which information is relevant to the hospital, clinic, or laboratory in which sample gathering and/or data generation was conducted. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a subject data section, including subject medical history as well as administrative subject data (that is, data that are not essential to the diagnosis, prognosis, or treatment assessment) such as information to identify the subject (e.g., name, subject date of birth (DOB), gender, mailing and/or residence address, medical record number (MRN), room and/or bed number in a healthcare facility), insurance information, and the like), the name of the subject’s physician or other health professional who ordered the susceptibility prediction and, if different from the ordering physician, the name of a staff physician who is responsible for the subject’s care (e.g., primary care physician).

The report may include a sample data section, which may provide information about the biological sample analyzed, such as the source of biological sample obtained from the subject (e.g. blood, type of tissue, etc.), how the sample was handled (e.g. storage temperature, preparatory protocols) and the date and time collected. Report fields with this information can generally be populated using data entered by the user, some of which may be provided as pre-scripted selections (e.g., using a drop-down menu).

The report may include an assessment report section, which may include information generated after processing of the data as described herein. The interpretive report can include a prognosis of the likelihood that the patient will develop preeclampsia. The interpretive report can include, for example, results of the analysis, methods used to calculate the analysis, and interpretation, i.e. prognosis. The assessment portion of the report can optionally also include a Recommendation(s). For example, where the results indicate the subject’s prognosis for time to onset of labor.

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc.

It will be readily appreciated that the report can include all or some of the elements above, with the proviso that the report generally includes at least the elements sufficient to provide the analysis requested by the user (e.g., a diagnosis, a prognosis, or a prediction of responsiveness to a therapy).

### EXPERIMENTAL

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Integrated Trajectories of the Maternal Metabolome, Proteome, and Immunome Predict Labor Onset

Estimating the time of delivery is of high clinical importance because pre- and post-term deviations are associated with complications for the mother and her offspring. However, current estimations are inaccurate. As pregnancy progresses toward labor, major transitions occur in fetomaternal immune, metabolic, and endocrine systems that culminate in birth. The comprehensive characterization of maternal biology that precedes labor is key to understanding these physiological transitions and identifying predictive biomarkers of delivery. Here, a longitudinal study was conducted in 63 women who went into labor spontaneously. More than 7000 plasma analytes and peripheral immune cell responses were analyzed using untargeted mass spectrometry, aptamer-based proteomic technology, and single-cell mass cytometry in serial blood samples collected during the last 100 days of pregnancy. The high-dimensional dataset was integrated into a multiomic model that predicted the time to spontaneous labor[R = 0.85, 95% confidence interval (Cl) [0.79 to 0.89], P = 1.2 × 10₋₄₀, N = 53, training set; R = 0.81, 95% CI [0.61 to 0.91], P = 3.9 Å~ 10₋₇, N = 10, independent test set]. Coordinated alterations in maternal metabolome, proteome, and immunome marked a molecular shift from pregnancy maintenance to prelabor biology 2 to 4 weeks before delivery. A surge in steroid hormone metabolites and interleukin-1 receptor type 4 that preceded labor coincided with a switch from immune activation to regulation of inflammatory responses. Our study lays the groundwork for developing blood-based methods for predicting the day of labor, anchored in mechanisms shared in preterm and term pregnancies.

In this study, we combined an untargeted mass spectrometry approach with an aptamer-based technology to quantify the concentrations of 4846 metabolomic and proteomic analytes in longitudinally collected plasma samples during the 100-day period preceding spontaneous labor onset. In parallel, we used a single-cell mass cytometry immunoassay to quantify the dynamic changes in the distribution and intracellular signaling responses of all major innate and adaptive peripheral immune cells (2296 features). The analysis generated three high-dimensional omic datasets. We applied a stacked generalization (SG) algorithm to the multiomic dataset to build and independently validate an integrated model that predicted the time to labor (TL). Model component trajectories revealed precisely timed alterations that marked a transition from pregnancy maintenance to prelabor biology. Our findings and predictive modeling approach can serve to identify elements of a common pathway that precedes labor in term as well as pre- or postterm pregnancies.

### Results

Maternal metabolome, proteome, and immunome are assessed in the 100 days preceding the day of labor One-hundred twelve pregnant women receiving routine antepartum care at the Lucile Packard Children’s Hospital in Stanford, CA, USA, were enrolled during their second or third trimester of pregnancy. After exclusion of patients who did not meet the inclusion criteria, such as medical induction of labor (see Materials and Methods), an analysis was performed on samples from 53 patients (training cohort) with spontaneous labor contractions. The day of labor for this study is defined as the day of admission for spontaneous labor (contractions occurring at least every 5 min, lasting >1 min, and associated with cervical change). All patients in the training cohort were in stage I labor on the day of labor at the time of diagnosis, among which 73.6% were in the latent phase and delivered within 11 hours [interquartile range (IQR) [5, 18] hours]. The remaining 26.4% were in the active phase of labor and delivered within 4 hours (IQR [2, 10] hours). The difference between the day of labor and day of delivery ranged from 0 to 1 day (median = 0; SD = 0.23; range [0, 1] days). Five women in the training cohort delivered preterm [34 weeks + 0 days (34+0) to <37 weeks of gestation]. An independent analysis was performed on samples from 10 additional patients (test cohort), who had spontaneous labor (N = 5) or spontaneous rupture of membranes (N = 5). Patient demographics including labor and delivery, as well as ante- and peripartum parameters are shown in Table 1.

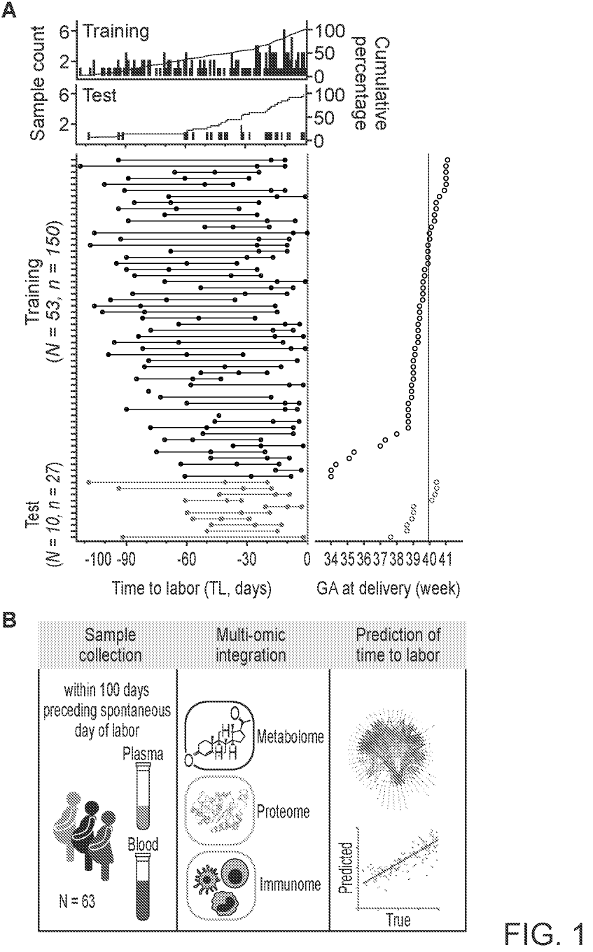

For each study participant, serial blood samples [median of three samples (plasma and whole blood) per patient, range [1, 3]] were collected during the last 100 days before labor (FIG. 1A). The approach leveraged the interindividual variabilities in sample collection time to define a continuous variable, the TL, which describes the difference between the day of sampling and the day of labor. In the aggregated sample cohort of all patients, the TL was distributed with near daily resolution across the last 100 days of pregnancy with a median time of blood sampling of 36 days (~5 weeks) before the day of labor. The plasma concentration of 3529 metabolites and 1317 proteins were quantified using a high-throughput untargeted mass spectrometry and an aptamer-based proteomic platform, respectively (FIG. 1B). Using a 46-parameter mass cytometry assay (table S1), a total of 2296 single-cell immune features were extracted from each sample including the frequencies of 41 immune cell subsets, representing major innate and adaptive populations, endogenous intracellular activities such as phosphorylation states of 11 signaling proteins, and capacities of each cell subset to respond to a series of receptor-specific immune challenges [lipopolysaccharide (LPS), interferon-α (IFN-α), granulocyte-macrophage colony-stimulating factor (GM-CSF), and a combination of interleukin-2 (IL-2), IL-4, and IL-6].

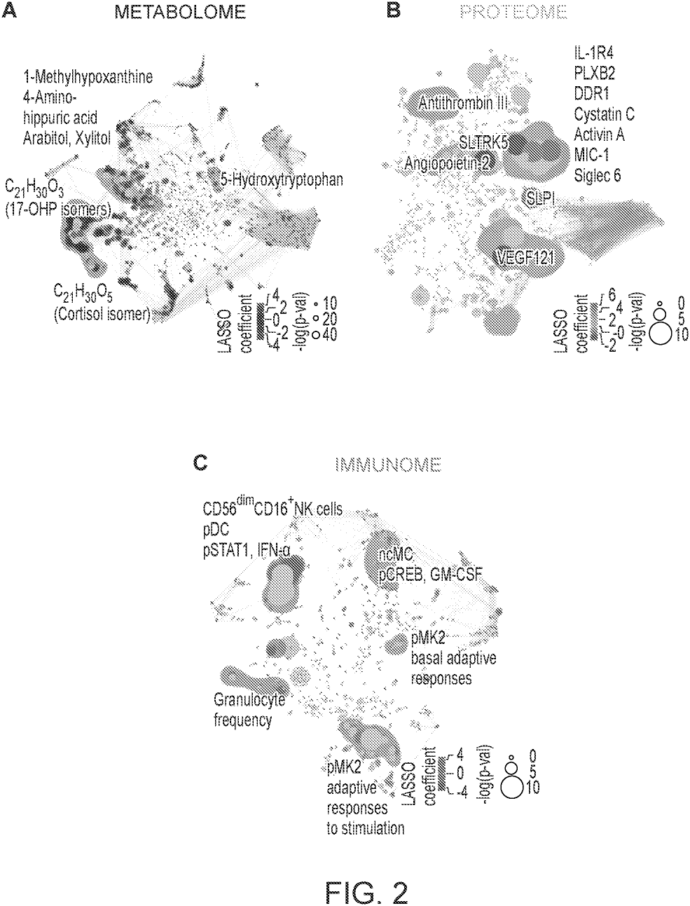

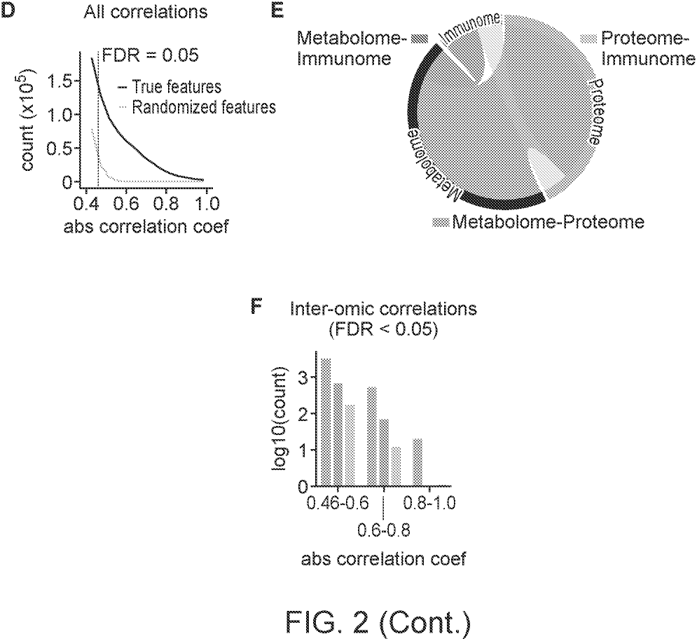

Multiomic modeling of the maternal interactome predicts labor onset The combined metabolome, proteome, and immunome datasets produced 7142 features per sample. Features were visualized with three correlation networks, highlighting intraomic (within-dataset) correlations across the last 100 days before the day of labor (FIG. 2, A to C). A single chord diagram highlighted interomic (between-dataset) correlations between features from two different datasets (FIG. 2, D and E), after controlling to a false discovery rate (FDR) of 0.05 (Spearman R > 0.46) computed from the distribution of correlation between randomly generated features (FIG. 2D). Individual biological systems were tightly orchestrated because 99% of all omic correlations were found in feature pairs belonging to the same dataset (FIG. 2, A to C). Correlations between the three biological systems included 3995 weak (R = 0.46 to 0.59), 596 moderate (R = 0.6 to 0.79), and 21 strong (R = 0.8 to 1.0) interomic correlations (FIG. 2, E and F), revealing an interactome of late pregnancy. Of all interomic correlations (R > 0.46, FDR < 0.05), 80% were observed between the metabolome and proteome, 4% between the immunome and proteome, and 16% between the immunome and metabolome (FIG. 2F). Overall, the multimodal analysis of plasma analytes and peripheral blood immune cells measured during pregnancy revealed a concerted behavior between the metabolomic, proteomic, and immunologic systems. The interactome analysis did not account for the timing of omic measurements, such that observed correlations were not enriched for interactions temporally linked to the time in pregnancy. However, the analysis highlighted the interconnected nature of the multiomic dataset, justifying the need for an integrated approach to identify biologically relevant components predictive of the TL.

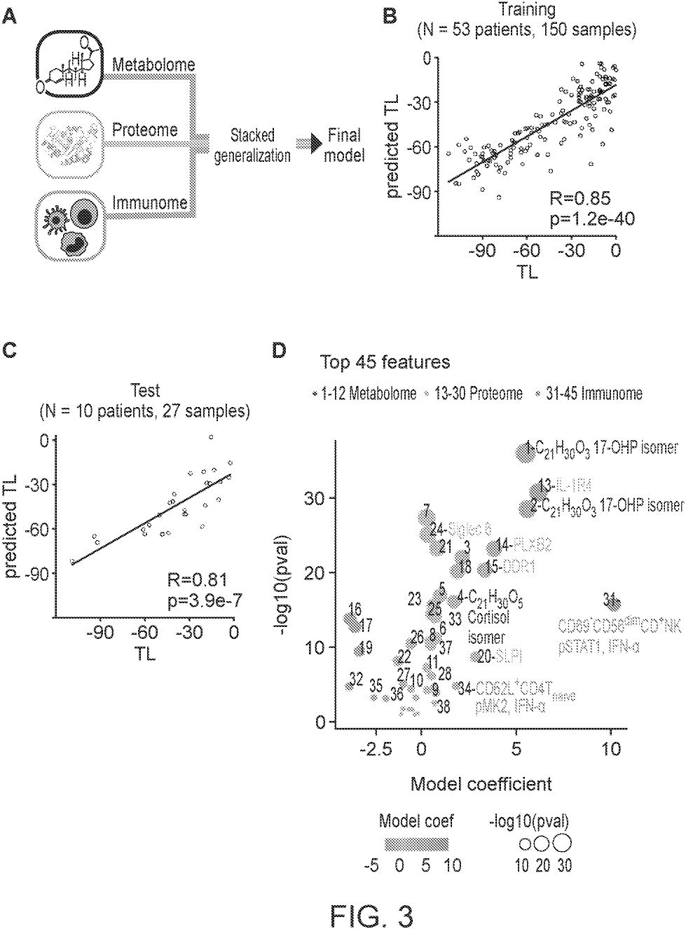

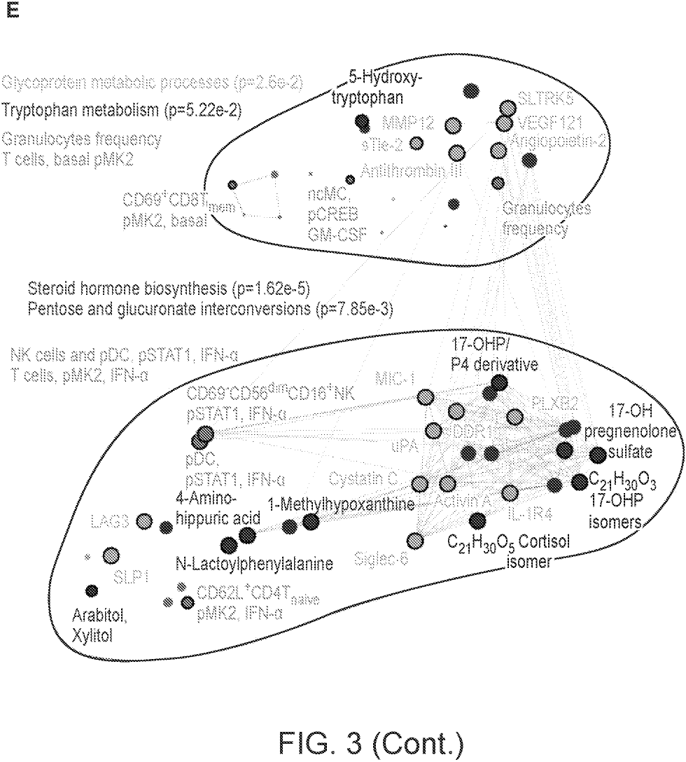

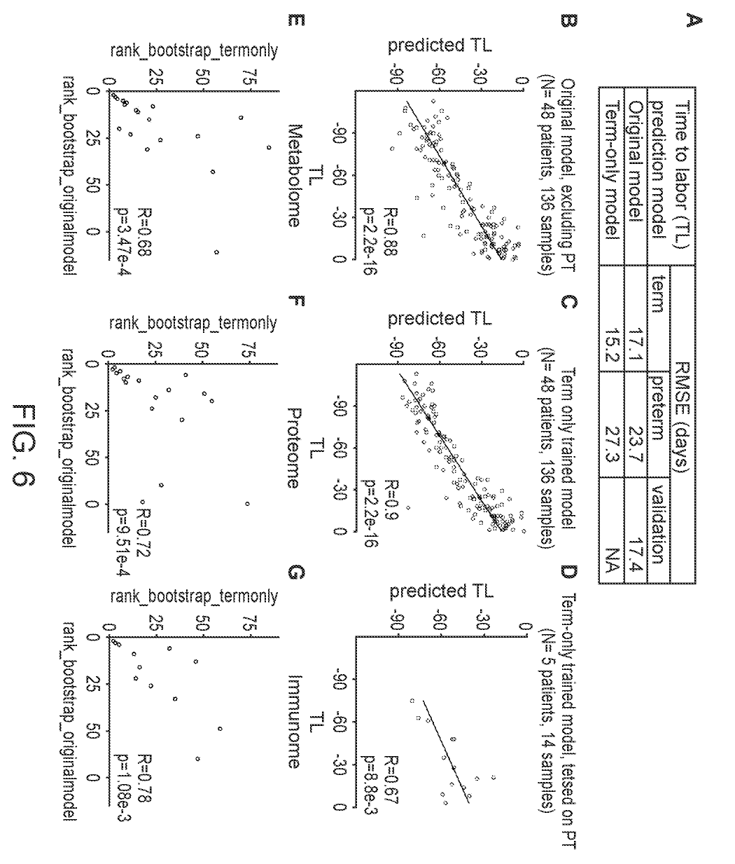

Peripheral blood metabolic, proteomic, and immunologic events informed an integrated approach to predict the TL. Here, multivariate least absolute shrinkage and selection operator (LASSO) linear regression models were first individually built for each omic dataset and then integrated into a single model by SG. An advantage of the SG method is that differences in size and modularity of individual omic modalities are accounted for to prevent datasets of higher dimensions (such as the metabolome) to overwhelm the integrated model (FIG. 3A). The SG model predicted the TL from the measurement of metabolic, proteomic, and immunologic features with high accuracy [R = 0.85, 95% confidence interval (Cl) [0.79 to 0.89], P = 1.2 × 10^-40, root mean square error (RMSE) = 17.7 days, N = 53] (FIG. 3B). Statistical significance was established using a cross-validation method that accounts for the high dimensionality of the data. The generalizability of the SG model was prospectively tested in an independent cohort of 10 additional women (R = 0.81, 95% CI [0.61 to 0.91], P = 3.9 × 10^-7, RMSE = 17.4 days) (FIG. 3C). Five of the 53 patients included in the training cohort experienced spontaneous preterm labor (GA at delivery < 37 weeks). Although comparing term and preterm labor was not a primary aim of the study, the presence of these five patients questioned whether the integrated SG approach would generalize to predict the TL when labor occurred preterm. A new model trained on a subcohort of patients with term labor successfully predicted the TL for patients with preterm labor (R = 0.67, 95% CI [0.21 to 0.86], P = 8.8 × 10^-3, RMSE = 27.3 days; FIG. 6, A to D). In addition, there was a strong overlap between the most informative features of the original model (including term and preterm patients) and the term-only model (FIG. 6, E to G; R = 0.68 to 0.78, Spearman correlation in bootstrap feature ranking between the two models). The results suggest that the SG model generalized to the prediction of the TL for both term and preterm labor. The data also confirm that the SG model built on the entire patient cohort was not driven by a preterm-specific prelabor biology. A confounder analysis further established that the prediction accuracy was not influenced by other clinical or demographic variables [including race, body mass index (BMI), and major comorbidities; table 3]. In summary, our assessment of maternal circulating factors in the peripheral blood provided an accurate prediction for the timing of labor that was independent of the GA based on EDD.

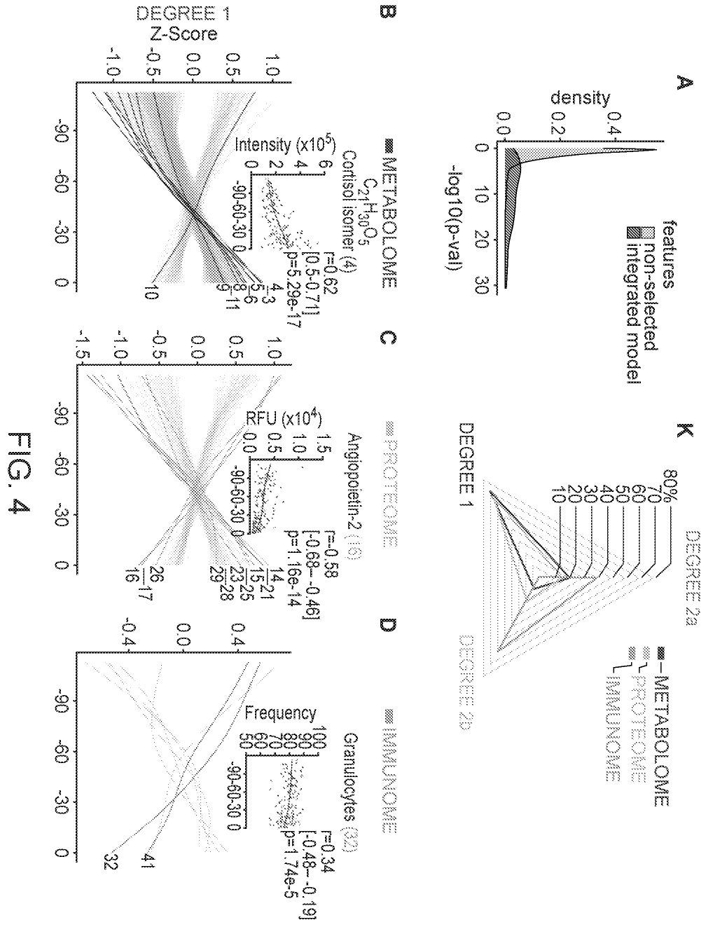

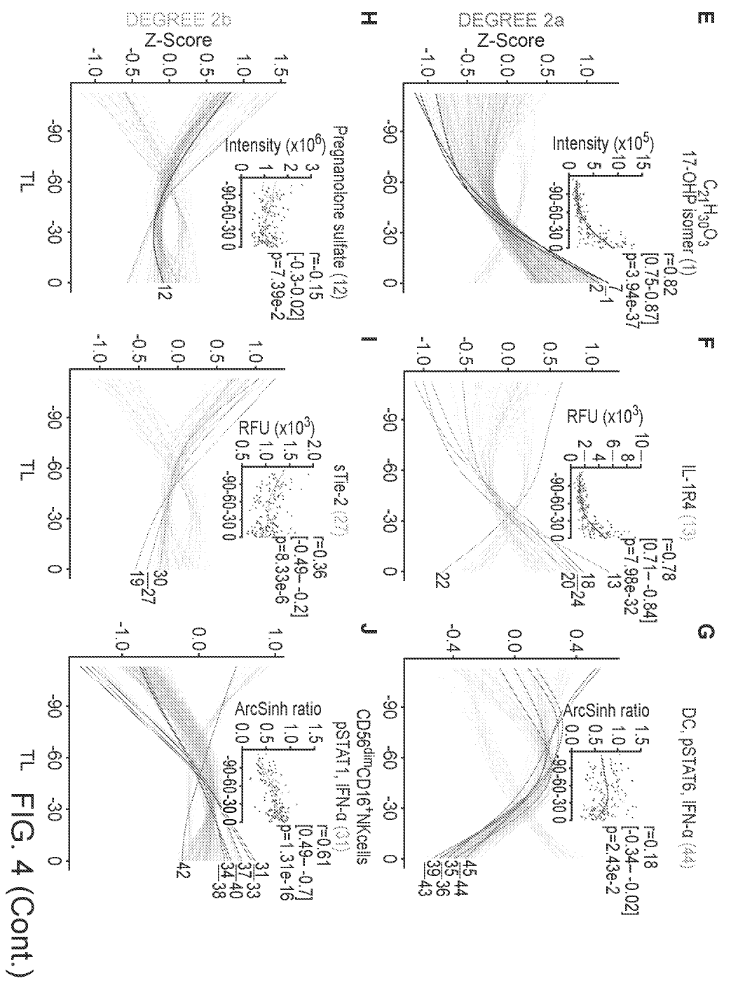

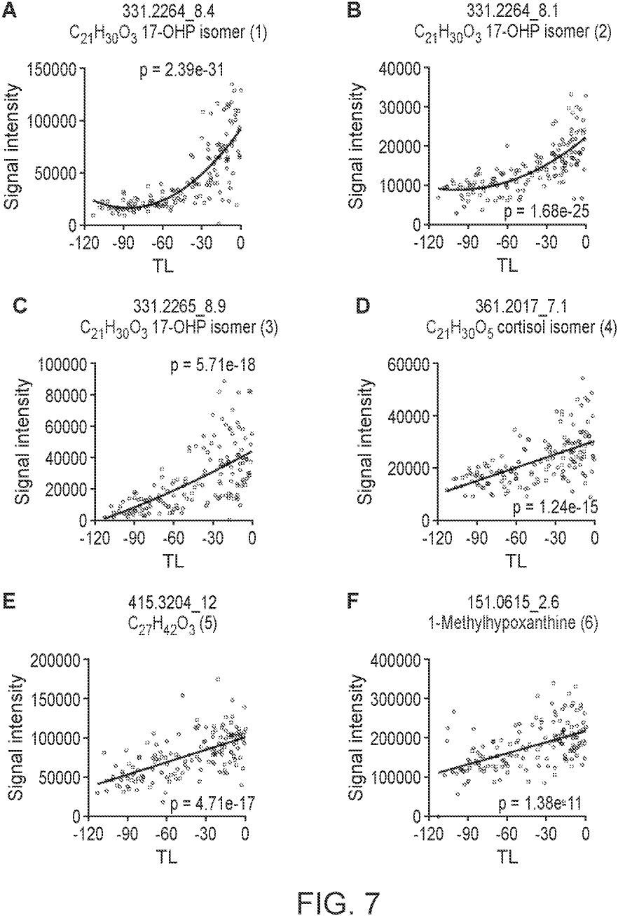

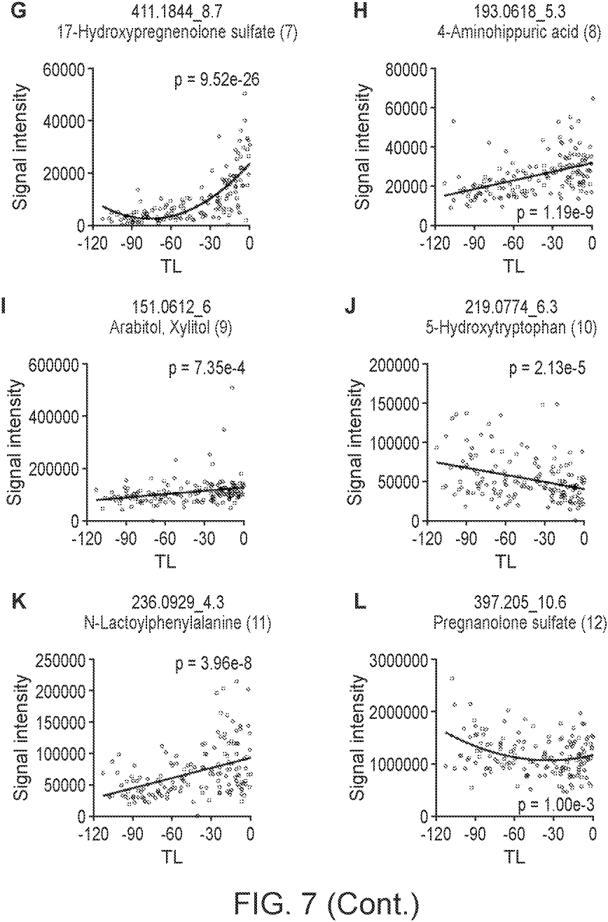

Trajectories of metabolome, proteome, and immunome reveal alterations in prelabor dynamics. To facilitate the biological interpretation of the multivariate SG model, we focused on the model features that contributed most to the prediction of the TL (selected using a bootstrap and ranking approach; see Materials and Methods). These features included 12 metabolomic, 18 proteomic, and 15 immune cell features (FIG. 3D and table 4) and formed a correlation network that segregated into two clusters (FIG. 3E). For each cluster, enriched proteomic and metabolomic pathways were identified using the Fisher’s test and the hypergeometric test, respectively. The lower cluster was enriched for metabolic features representing steroid hormone biosynthesis, and pentose and glucuronate interconversions (carbohydrate metabolism) that clustered with innate and adaptive immune cell responses to IFN-α stimulation [including phosphorylated signal transducer and activator of transcription 1 (pSTAT1) and phosphorylated mitogen-activated protein kinase-activated protein kinase (pMK2) in dendritic cells (DCs), natural killer (NK) cells, and T cell subsets] (FIG. 3E). The upper cluster contained metabolic features enriched for tryptophan metabolism and proteins representing glycoprotein metabolic pathways that clustered with various immune cell features, including granulocyte frequencies, signaling responses to GM-CSF in nonclassical monocytes (ncMCs) and basal pMK2 signaling in T cell subsets (FIG. 3E). The pathway enrichment analysis provided a snapshot of key biological systems temporally linked to the TL. To examine the dynamic behavior of biological events predictive of the TL, individual model features were plotted over time (FIG. 4, FIGS. 7 to 9, and table 4). Classifying the dynamic behavior of each feature revealed three general trajectory patterns on the basis of the goodness of fit of a pattern-fitting model (FIG. 4A and table 5): linear progression model (FIG. 4A and table 5): linear progression (degree 1; FIG. 4, B to D) or quadratic progression, including accelerating (surging of an increasing or decreasing pattern over time) (degree 2a; FIG. 4, E to G) or decelerating (plateauing of an increasing or decreasing pattern over time) (degree 2b; FIG. 4, H to J) progression (table 5). We plotted the distribution of trajectory patterns across all datasets. The resulting plot (FIG. 4K) showed a remarkable overlap of the behavior of metabolomic and proteomic model features, which were predominantly classified as degree 1 (constant rate). In contrast, immune cell trajectories predominantly followed a degree 2b (decelerating) pattern.

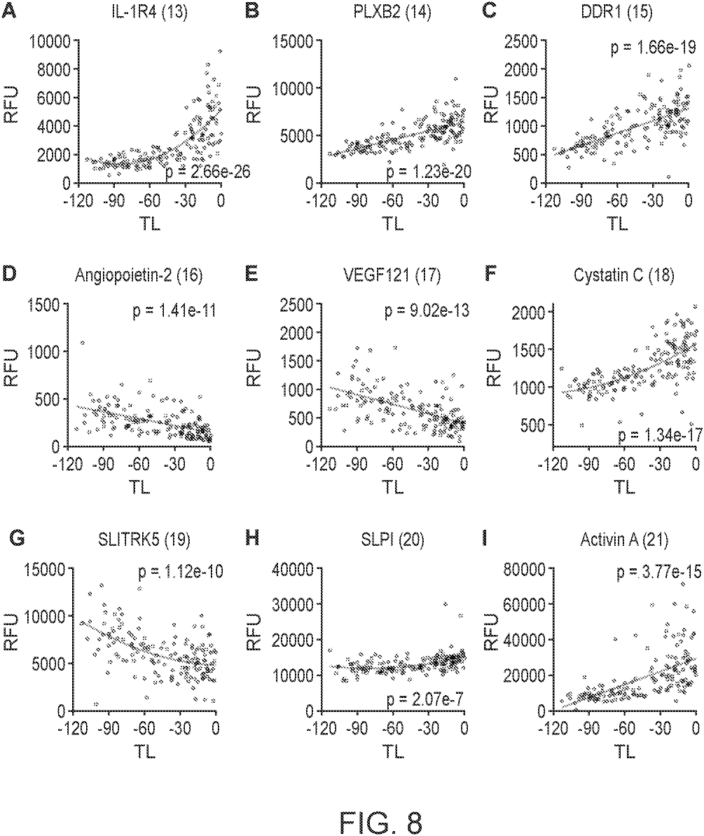

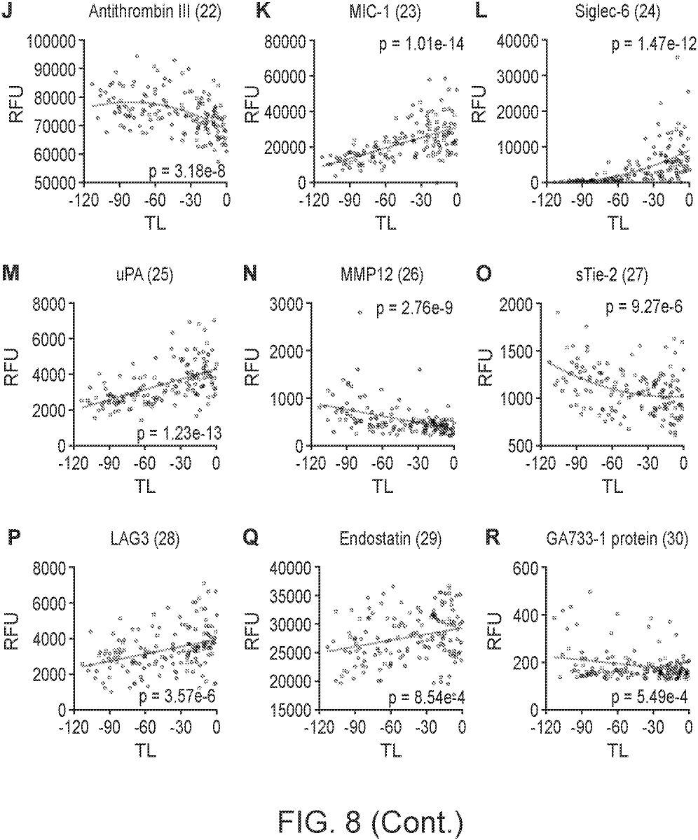

Degree 1 trajectories highlighted biological processes progressing linearly throughout the last 100 days of pregnancy until labor (FIG. 4, B to D). For example, the plasma concentration of an isomer of cortisol, strongly correlating with cortisol (Spearman R = 0.7; Materials and Methods), increased steadily from TL -100 to labor onset (FIG. 4B), recapitulating known steroid changes occurring throughout pregnancy. Similarly, proteins expressed by fetal membranes constantly increased throughout the 100 days before labor, such as plexin-B2 (PLXB2) and discoidin domain receptor-1 (DDR1) (FIG. 8). In contrast, Angiopoietin-2, a protein that contributes to placental vascular development, decreased at a constant rate throughout the study period (FIG. 4C).

In addition to the many proteomic and metabolomic model features with constant pattern trajectories, accelerating (degree 2a) or decelerating (degree 2b) trajectories denoted important prelabor alterations of the maternal metabolome and proteome (FIG. 4, E, F, H, and I). Among the most informative degree 2 metabolomic features were isomers of 17-hydroxyprogesterone (17-OHP) and 17-hydroxypregnenolone sulfate, an upstream substrate for the production of 17-OHP. The isomers of 17-OHP correlate with 17-OHP, suggesting that they have similar biological functions and belong to similar pathways (Materials and Methods). Plasma concentrations of these features increased in accelerated fashion within the last 30 days before the day of labor (FIG. 4E and FIG. 7). Whereas this finding confirms known progesterone biology in the late third trimester, our data provide additional temporal information showing that a surge in 17-OHP, one of the most informative features of the predictive model, is tightly linked to the timing of labor. Furthermore, metabolites with degree 2b trajectories included pregnenolone sulfate, which showed decelerating behavior, stagnating around 30 days before the day of labor (FIG. 4H).

Among the most informative degree 2 proteomic features of the predictive model were trajectories whose accelerating or decelerating patterns pointed toward important prelabor alterations in placental biology, coagulation, and inflammation. The most informative degree 2a proteomic feature was IL-1 receptor type 4 (IL-1 R4), the soluble inhibitory receptor of the proinflammatory cytokine IL-33. IL-1R4 plasma concentration surged during the last 30 days before labor (FIG. 4F and FIG. 8). The data complement prior studies showing an elevated concentration of IL-1R4 during the third trimester of pregnancy. Surging concentrations of IL-1R4 observed in the systemic circulation may counteract the proinflammatory effects of IL-33, potentially released upon mechanical uterine distension and in the context of the local inflammation occurring at the fetomaternal interface. Hence, IL-1R4 may be an important regulator of inflammation during the late phase of pregnancy.

Also surging with approaching labor (degree 2a) were two proteins highly expressed by the placenta, Activin-A and sialic acid binding immunoglobulin-like lectin-6 (Siglec-6) (FIG. 8). In contrast, the trajectory of antithrombin III (ATIII), an endogenous anticoagulant, negatively accelerated during the last 30 days before the day of labor (FIG. 8). Soluble tunica interna endothelial cell kinase-2 (sTie-2), a regulator of angiopoietin availability for vasculogenesis, displayed a decelerating trajectory (FIG. 4I). Overall, together with the constantly rising concentrations of fetal membrane-derived PLXB2 and DDR1 (FIG. 8), the coordinated trajectories of angiogenic factors sTie-2, Angiopoietin-2 (FIG. 4C), and vascular endothelial growth factor 121 (VEGF121) as well as Activin-A, and Siglec-6 (FIG. 8) suggest that these proteins are integral components of a plasma fetoplacental signature that portends the impending day of labor.

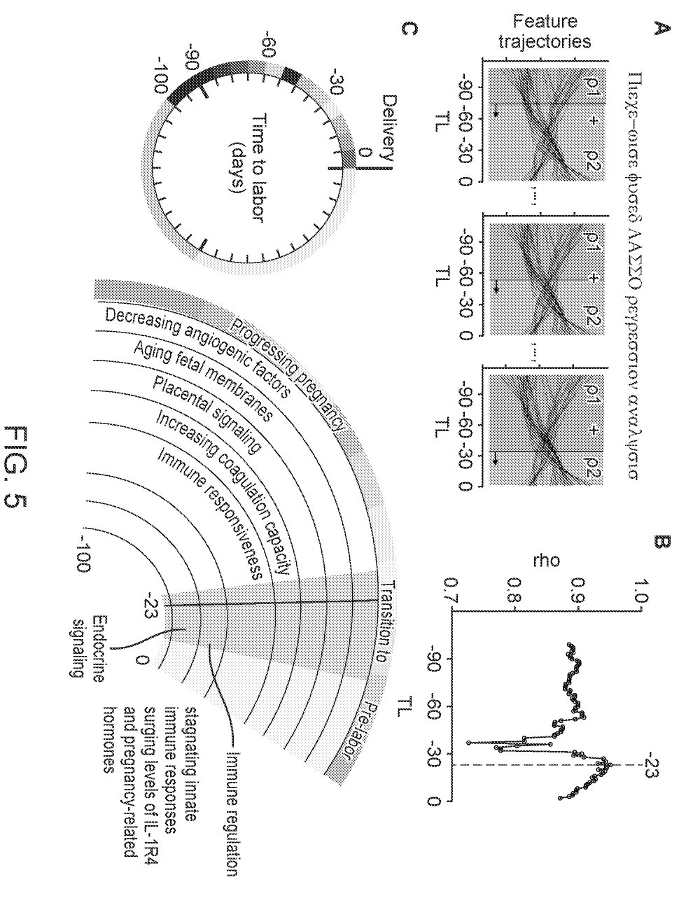

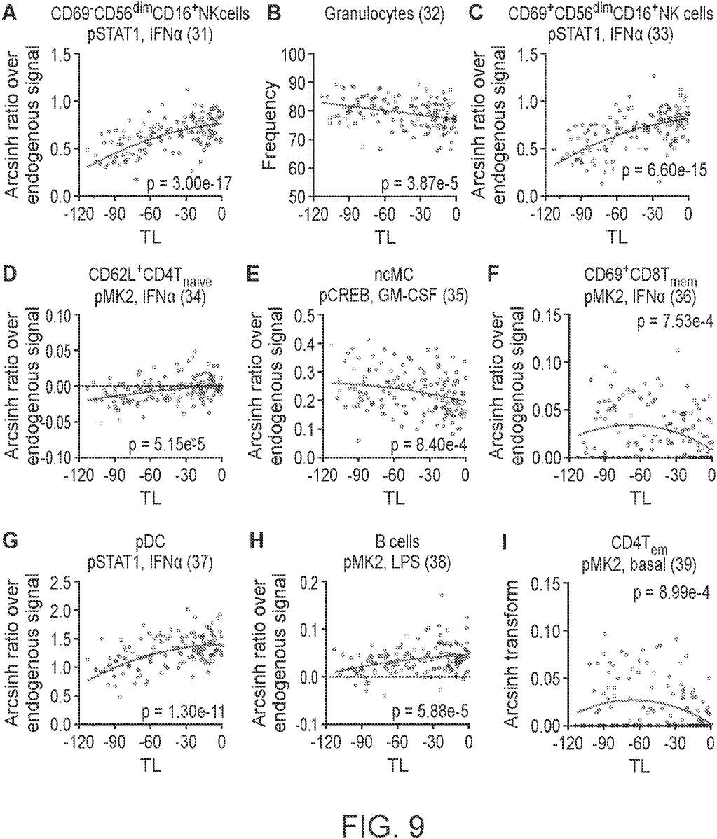

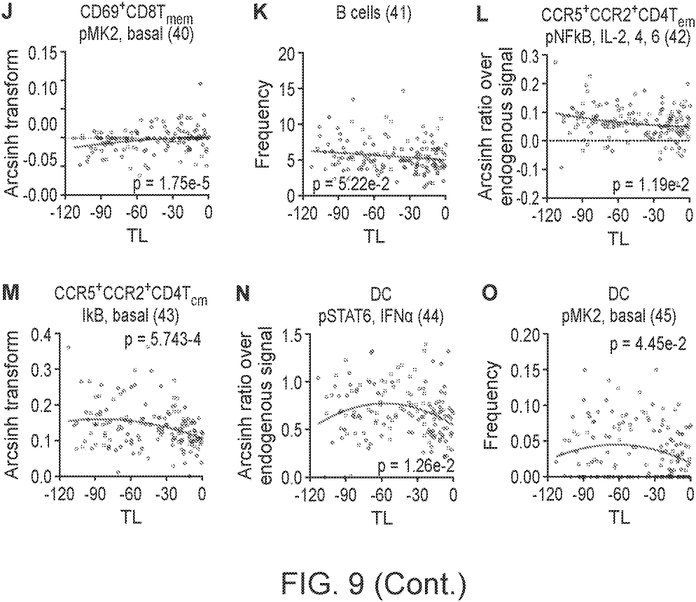

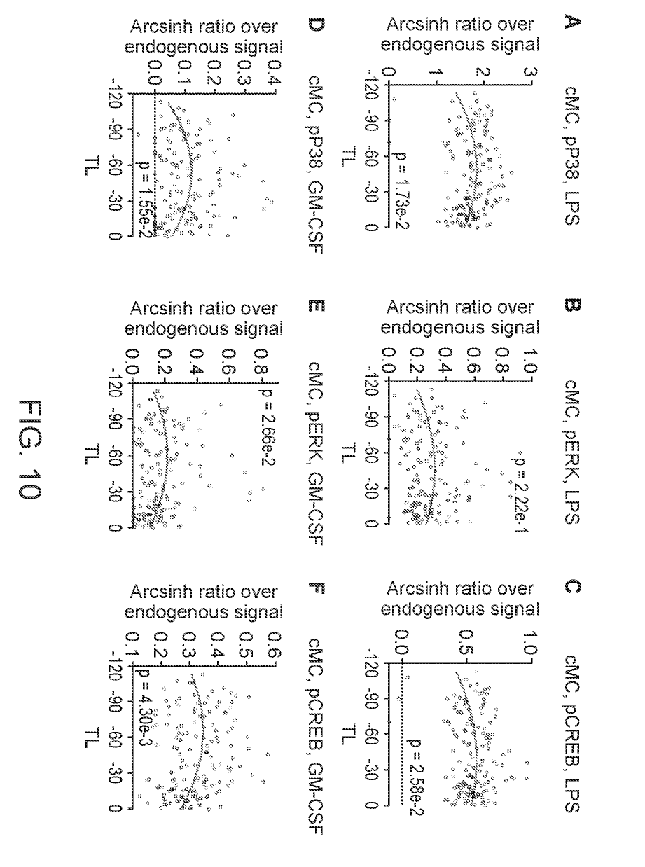

Plasma metabolites and proteins form the interactive environment for circulating immune cells. Immune cell trajectories predominantly followed a decelerating pattern (FIG. 4, D, G, and J), in contrast to accelerating or constantly increasing plasma analyte trajectories (FIG. 4K). Granulocyte frequencies decreased over time (FIG. 4D). In parallel, decelerating signaling trajectories were observed along the Janus kinase (JAK)-STAT and myeloid differentiation primary response 88 (MyD88) signaling pathways in both innate and adaptive immune cells (FIG. 9). This decelerating behavior was particularly pronounced in innate immune cells, as illustrated by the pSTAT1 signal in CD56dimCD16+ NK cells and the pSTAT6 signal in DCs in response to IFN-α (FIG. 4, G and J), the phosphorylated cyclic adenosine monophosphate response element-binding protein (pCREB) response in ncMCs in response to GM-CSF (FIG. 5), and the phosphorylated P38 mitogen-activated protein kinase (pP38), phosphorylated extracellular signal-regulated kinase (pERK) and pCREB signals in classical monocytes (cMCs) in response to LPS and GM-CSF (FIG. 10). During the 100 days preceding labor, proinflammatory innate immune cell responses first increased, in accordance with their previously described trajectory during the first and second trimesters, and then stagnated or decreased closer to the day of labor. These findings indicate a regulated dampening of systemic immune cell responses before the day of labor that may counterbalance the local inflammatory environment emerging at the fetal membranes, cervix, and fetomaternal interface during labor and parturition.

A breakpoint defined by nonlinearity of omic trajectories demarcates a transition from pregnancy to prelabor biological adaptations. The presence of degree 2 (quadratic) trajectories across all omic datasets pointed toward a period of disruption with approaching labor that resonated across all measured biological systems (FIG. 4, E to J). Identifying the timing of such a nonlinear transition is clinically relevant because it defines when the assessment of peripheral blood analytes is linked to prelabor biology rather than a reflection of the biology relevant for the maintenance of pregnancy. A piecewise fused LASSO regression analysis was used to provide an estimate as to when before the day of labor such a transition occurs (FIG. 5A). This approach (see Materials and Methods) combined the predictions rho (ρ) of two LASSO regression models built using the data points before or after a given TL threshold, while varying the threshold across all time points. A maximum ρ value was reached when the models on each side of the threshold contained distinct yet top informative biological features that, when combined, reached maximal predictive accuracy. The piecewise fused LASSO regression analysis produced a maximum at 23 days before the day of labor (range [-27, -13] days; ρ at -23 days = 0.95; FIG. 5B).

Our results indicate that the maternal metabolome, proteome, and immunome undergo a marked transition from maintenance of pregnancy to a phase of prelabor biology that is linked to the timing of labor (FIG. 5C). The model in FIG. 5C summarizes major characteristics of the biology before and after the transition period occurring 2 to 4 weeks before labor.

This study combined the high-content assessment of circulating plasma factors with single-cell analyses of peripheral immune cells to survey dynamic changes in the maternal metabolome, proteome, and immunome preceding the day of labor. Using a stringent analytical method that accounts for the dimensionality and heterogeneity of the data, we built and independently validated a multiomic model that predicted the timing of spontaneous labor. Current efforts to monitor biological adaptations during pregnancy have primarily been focused on investigating dynamics that differentiate normal from pathological pregnancies on the basis of GA. Although they are informative in characterizing pathological deviations during pregnancy, these approaches provide limited information for the prediction of timing of labor because they rely on estimates of GA, which are imposed by human assumption rather than based on biological determination. In contrast, our study paradigm did not use an estimate but an observed outcome, such as the time to spontaneous labor. This outcome was independent of GA on the basis of EDD and accounted for the inherent variations in pregnancy duration. Hence, our approach enabled characterization of labor-relevant pathways, which may be important for the identification of labor-specific mechanisms in normal and pathological pregnancies.

In our study, the analysis of metabolic, proteomic, and immunologic trajectories provided an integrated view of response mechanisms associated with the TL. Two major themes evolved: (i) The coordinated adaptations across biological systems revealed a pre-labor interactome that pointed toward cross-talk between circulating plasma factors and immune cell responses toward the end of pregnancy, and (ii) dynamics in omic trajectories uncovered a marked transition from pregnancy maintenance to pre-labor biology 2 to 4 weeks before delivery.

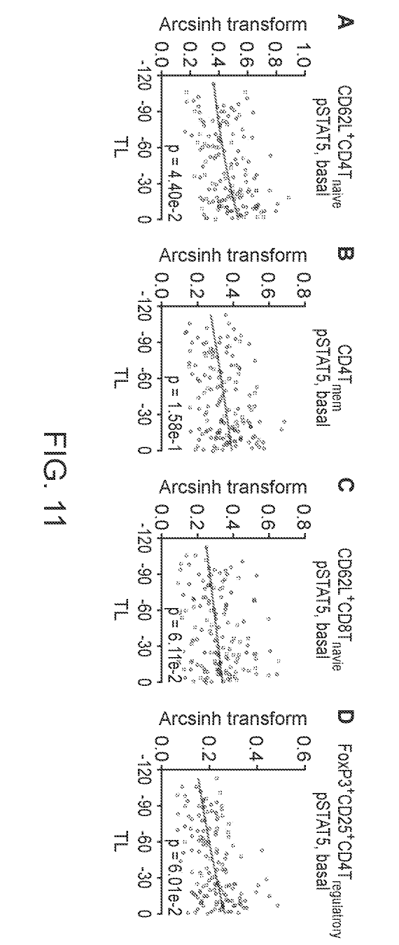

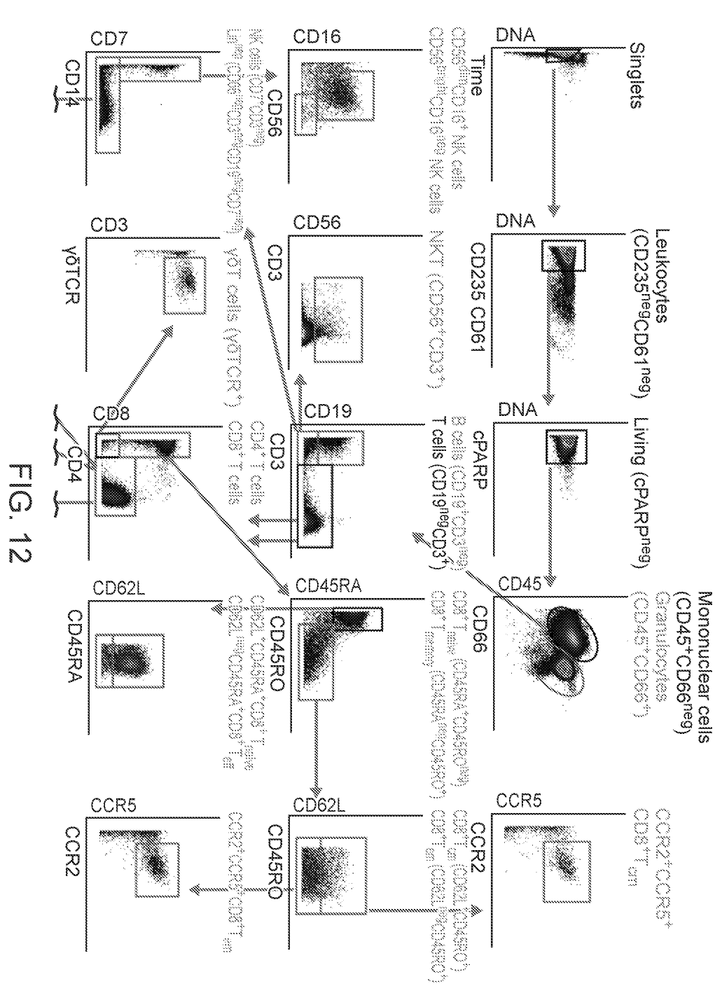

Aspects of our analysis agree with prior studies of endocrine and inflammatory changes during late pregnancy. For example, steroid hormone metabolites were among the most informative metabolic features of the predictive model, which is consistent with the established role of progesterone in the maintenance of mammalian pregnancy and the progression to labor. Similarly, key immune response features of our predictive model are consistent with previous studies reporting on peripheral immune activation with approaching labor. Specifically, the pSTAT1 signaling in CD56_{dim}CD16+ NK cells in response to IFN-α was a key immune feature common to our current model predicting the TL and our previous model predicting GA. In contrast, the endogenous STAT5 signaling activity in T cells, important for predicting GA, did not contribute to the current model (FIG. 11). Differences in immune features between the two models likely reflect the dynamic evolution of immune signatures throughout pregnancy and underpin that the selection of predictive parameters depends on the window of observation.

Our results also suggest previously undescribed cross-talk between metabolic, proteomic, and immune cell features that precedes the onset of labor. We found that the surge in steroid hormone metabolites 2 to 4 weeks before labor coincided with dynamic changes in plasma protein concentrations and immune cell responses that reflected a previously unrecognized switch from immune activation to regulation of inflammatory responses. One of the most pronounced examples of immune regulation was the surge in the concentration of IL-1R4, which paralleled the dampening of JAK-STAT and MyD88 responses in innate immune cells (FIG. 4, F and J). The data suggest that IL-1R4, an IL-33 antagonist, may play a prominent regulatory role during the prelabor phase by neutralizing IL-33, a proinflammatory yet regulatory T cell-stabilizing alarmin released upon tissue remodeling. In mice, IL-33 has been assigned a pregnancy-maintaining role. Rising concentrations of IL-1R4 in response to increased IL-33 activity could function as a labor-initiating signal by disrupting IL-33-mediated mechanisms of fetomaternal tolerance, while simultaneously counteracting systemic proinflammatory innate responses to accumulating circulating fetal material with approaching parturition. The observed dampening of systemic inflammatory events with approaching labor contrasts with prior studies showing increased local inflammation of the cervix, decidua, fetal membranes, and placenta during labor and parturition, although several other studies of systemic immune responses during pregnancy echo our findings of decreased peripheral immune cell responses with approaching labor. Hence, the systemic dampening of proinflammatory responses may be important to keep in check proinflammatory events initiated locally with labor onset.

Our multiomic analysis also provided a high-resolution fingerprint of essential biological processes preceding the day of labor, including changes in vascular development, placental biology, and fetal membrane activation. Angiogenic factors, potentially involved in placental vascularization, progressively diminished; whereas coagulation capacity became enhanced. In accordance with previous studies, placental factors Activin-A and Siglec-6 followed accelerated trajectories, potentially reflecting placental aging. Increased concentrations of serum Activin-A and placental Siglec-6 are also detected in labor versus nonlabor deliveries. In addition, the steady increase of epithelial factors PLXB2 and DDR1 likely mirrors the remodeling of the fetal membranes. Several of these circulating proteins, including Angiopoietin-2, urokinase-type plasminogen activator (uPA), Activin-A, Siglec-6, PLXB2, and DDR1, have proven to be informative model components for the prediction of GA, both by our group and others. The pathway enrichment analysis of the most informative model features provided further insight on previously undescribed biological pathways implicated in the transition from progressing pregnancy to labor onset. First, glycoproteins and proteins associated with glycoprotein metabolism, including ATIII, VEGF121, matrix metalloproteinase 12 (MMP12), Angiopoietin-2, sTie-2, and SLIT and NTRK-like protein 5 (SLITRK5), were enriched among the proteomic features. SLITRK5 has a high affinity for pregnancy-specific glycoprotein, an immune tolerance-enhancing protein released from the placenta and peaking in late gestation. Second, metabolic pathways, including tryptophan metabolism, and pentose/glucuronate interconversions (carbohydrate metabolism) were also enriched. The systemic concentration of serotonin-precursor 5-hydroxytryptophan is a proxy for serotonin activity in the central nervous system and facilitates vasoconstriction in the placenta. The involvements of glycoproteins, vasoactive neurotransmitters, and energy metabolism highlight prelabor dynamics beyond previously described fetal and immunoendocrine mechanisms.

The cohort included a homogeneous population of women recruited at a single center, who went into labor predominantly at term (N = 63; of which five preterm < 37 weeks, zero postterm > 42 weeks). Hence, our model predicted the TL in term and preterm pregnancies with similar accuracy. Studies specifically focusing on women with preterm labor are particularly important because the ability to predict labor several weeks before the actual day of labor provides a critical time window that would aid in clinical decision-making for the early management of a patient at risk of preterm labor. Our approach provided highly informative results regarding multiomic adaptations relevant to the TL. On the basis of prior biological knowledge, particular associations between proteins or metabolites and immune cell responses can be hypothesized to be biological interactions.

In summary, determining the timing of labor and delivery, and predicting preterm and postterm pregnancy risk, is an important clinical challenge. The biological insights of this study guide therapeutic approaches to extend pregnancy when the labor signature is detected early (preterm birth) or to accelerate labor processes to avoid the need for induction of labor in postdate pregnancies. The results provide a means to examine prelabor biology for the development of a universal diagnostic tool that can predict the TL.

### Materials and Methods

Study design The aim of this observational study was to determine a precise chronology of pregnancy-related metabolomic, proteomic, and immunologic adaptations in venous blood samples collected serially during the last 100 days of pregnancy. The study was conducted at the Lucile Packard Children’s Hospital (Stanford, CA, USA) and approved by the Institutional Review Board (approval ID, 40105). All participants signed an informed consent. Healthy pregnant women receiving routine antepartum care were eligible for the study if they were within 18 to 50 years of age, had a BMI < 40 in their second or third trimester of pregnancy as determined by their clinician using LMP and ultrasound estimates of GA, and had no immune-modifying comorbidities or medication usage. Participants were followed longitudinally until parturition, collecting one to three blood samples throughout the third trimester. In total, 112 women were recruited to meet the predetermined sample size required for sufficient power in this longitudinal study. Participants for whom labor was medically induced (N = 43) or who underwent cesarean section without labor (N = 4) were excluded. Two participants dropped out of the study. Participants with singleton pregnancies, who went into spontaneous labor (N = 53 training cohort, N = 5 test cohort) or experienced spontaneous rupture of membranes before labor onset (N = 5 test cohort) were included in the analysis. The day of labor was defined as day of admission for spontaneous labor (contractions occurring at least every 5 min, lasting >1 min, and associated with cervical change). For five patients from the test cohort, the day of spontaneous rupture of membrane was designated as the day of labor because labor would have likely ensued spontaneously, but modern clinical care required induction of labor for these patients. The GA at day of sampling was based on the clinical EDD established by LMP and/or ultrasonographic assessment according to the American College of Obstetricians and Gynecologists committee opinion. Researchers conducting the analyses were not blinded. Randomization was not applicable to this study. Demographics, pregnancy characteristics, and comorbidities for the 63 participants included in the analysis are summarized in Table 1.

Mass Cytometry. Ex vivo whole-blood immuno-assay. Whole blood was collected from study subjects and processed within 60 min after blood draw. Individual aliquots were stimulated for 15 min at 37° C. with LPS (1 ug/mL, InvivoGen, San Diego, CA), IFN-α (100 ng/mL, PBL Assay Science, Piscataway, NJ), GM-CSF (100 ng/mL, R&D Systems, Minneapolis, MN), and a cocktail of IL-2, -4, and -6 (each 100 ng/mL, R&D Systems) or left unstimulated. Samples were processed using a standardized protocol for fixing with proteomic stabilizer (SMART TUBE, Inc., San Carlos, CA) and stored at -80° C. until further processing.

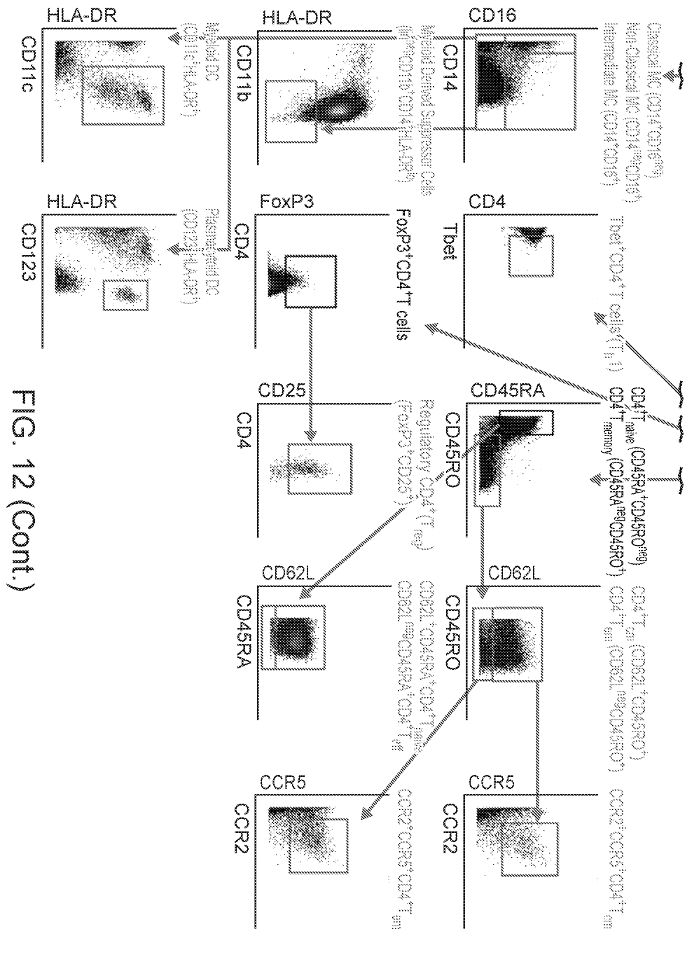

Mass cytometry and derivation of cell frequency, basal intracellular signaling, and intracellular signaling response features Forty-one innate and adaptive immune cell subsets were identified using a 45-parameter mass cytometry antibody panel and according to the gating strategy in FIG. 12. Cell frequencies were expressed as a percentage derived from singlet live mononuclear cells (DNA+cPARP-CD235-CD61-CD66-) except for granulocyte frequencies, which were expressed as a percentage of singlet live leukocytes (DNA+cPARP-CD235-CD61-). Endogenous intracellular signaling activities at the basal, unstimulated state were quantified per single cell for pSTAT1, pSTAT3, pSTAT5, pSTAT6, pCREB, pMK2, pERK, phosphorylated S6 ribosomal protein (prpS6), pP38, and phosphorylated nuclear factor κB (pNF-κB), and total inhibitor of NF-κB (IκB) using an arcsinh-transformed value calculated from the median signal intensity. Intracellular signaling responses to stimulation were reported as the difference in arcsinh-transformed value of each signaling protein between the stimulated and unstimulated conditions (arcsinh ratio over endogenous signal). A knowledge-based penalization matrix was applied to intracellular signaling response features in the mass cytometry data based on mechanistic immunological knowledge, as previously described. Mechanistic priors used in the penalization matrix are independent of immunological knowledge related to pregnancy or the day of labor.

Sample barcoding and minimization of experimental batch effect To minimize the effect of experimental variability on mass cytometry measurements between serially collected samples, samples corresponding to the entire time series collected from one woman were processed, barcoded, pooled, stained and run simultaneously. To minimize the effect of variability between study participants, sample sets of two women were run per day and the run was completed within consecutive days, while carefully controlling for consistent tuning parameters of the mass cytometry instrument (Helios CyTOF, Fluidigm Inc., South San Francisco, CA).

Antibody staining and mass cytometry The mass cytometry antibody panel included 28 antibodies that were used for phenotyping of immune cell subsets and 11 antibodies for the functional characterization of immune cell responses (table 2). Antibodies were either obtained preconjugated (Fluidigm, Inc.) or were purchased as purified, carrierfree (no BSA, gelatin) versions, which were then conjugated inhouse with trivalent metal isotopes utilizing the MaxPAR antibody conjugation kit (Fluidigm, Inc.). After incubation with Fc block (Biolegend), pooled barcoded cells were stained with surface antibodies, then permeabilized with methanol and stained with intracellular antibodies. All antibodies used in the analysis were titrated and validated on samples that were processed identically to the samples used in the study. Barcoded and antibody-stained cells were analyzed on the mass cytometer.

Identification of immune cell subsets The mass cytometry data was normalized using Normalizer v0.1 MATLAB Compiler Runtime (MathWorks). Files were then de-barcoded with a single-cell MATLAB debarcoding tool. Manual gating was performed using CellEngine (https://immuneatlas.org/#/) (Primity Bio, Fremont, CA). The following cell types were included in the analysis: Granulocytes, B cells, Natural Killer cells (CD3₋CD7₊), CD56_{bright}CD16₋NK, CD56_{dim}CD16₊NK (CD69₋ and CD69₊), TCRγδ T cells, CD4₊ T cells, CD4Tₙₐᵢᵥₑ (CD45RA₊CD45RO₋), CD62L+CD4Tₙₐᵢᵥₑ, CD4T_{effector}_{(eff)} (CD45RA₊CD62L₋), CD4T_{memory}₍ₘₑₘ₎ (CD45RA₋CD45RO₊), CD69₊CD4Tₘₑₘ, CD4T_{central}_{memory}_{(cm)} (CD62L₊CD45RO₊), CCR5₊CCR2₊CD4T_{cm}, CD4T_{effector}_{memory}₍ₑₘ₎ (CD62L-CD45RO₊), CCR5₊CCR2₊CD4Tₑₘ, CD25₊ FoxP3₊CD4₊T cells (T_{reg}), CD4₊Tbet₊T cells (Tₕ1), CD8₊ T cells, CD8Tₙₐᵢᵥₑ (CD45RA₊CD45RO₋), CD62L₊CD8Tₙₐᵢᵥₑ, CD8T_{eff} (CD45RA₊CD62L₋), CD8Tₘₑₘ (CD45RA₋CD45RO₊), CD69₊CD8Tₘₑₘ, CD8T_{cm} (CD62L₊CD45RO₊), CCR5₊CCR2₊CD8T_{cm}, CD8Tₑₘ (CD62L₋CD45RO₊), CCR5₊CCR2₊CD8Tₑₘ, NKT cells (CD56₊CD3₊), CD14₊CD16-classical monocytes (cMCs), CD14₋CD16₊ non-classical MCs (ncMCs), CD14₊CD16₊ intermediate MCs (intMCs), CCR2₊cMC, CCR2₊intMC, CCR2₋ncMC, CD14₊CD11b₊HLA₋DRₗₒ myeloid-derived suppressor cells (MDSC), CD14₋CD16₋HLA-DR₊ dendritic cells (DC), myeloid DC (CD11c₊ mDC), and plasmacytoid dendritic cells (CD123₊ pDC).

Proteomics. Blood was collected into EDTA tubes, kept on ice, and centrifuged (1500 x g, 20 min) at 4° C. within 60 min. Separated plasma was stored at ⁻80° C. until further processing. The 200-µL plasma samples were analyzed by the Genome Technology Access Center (St. Louis, MO) using a highly multiplexed, aptamer-based platform capturing 1310 proteins (SomaLogic, Inc., Boulder, CO).The assay quantifies proteins over a wide dynamic range (> 8 log) using chemically modified aptamers with slow off-rate kinetics (SOMAmer reagents). Each SOMAmer reagent is a unique, high-affinity, single-strand DNA endowed with functional groups mimicking amino acid side chains. In brief, samples were incubated on 96-well plates with a mixture of SOMAmer reagents. Two sequential bead-based immobilization and washing steps were used to eliminate nonspecifically-bound proteins, unbound proteins, and unbound SOMAmer reagents from protein target-bound reagents. After eluting SOMAmer reagents from the target proteins, the fluorescently-labeled reagents were quantified on an Agilent hybridization array (Agilent Technologies, Santa Clara, CA). Data were normalized in 4 specific steps and according to assay data quality control procedures defined in the good laboratory practice quality system of SomaLogic, Inc. Normalization steps control for signal intensity biases introduced by differential hybridization efficiencies and the overall brightness of plates, collection protocol artifacts, and batch effects between different plates.

Untargeted metabolomics from plasma by liquid chromatography (LC)-MS Sample preparation and data acquisition Plasma samples were thawed on ice, prepared and analyzed randomly as previously described. Briefly, metabolites were extracted using 1:1:1 acetone:acetonitrile:methanol, evaporated to dryness under nitrogen and reconstituted in 1:1 methanol:water before analysis. Metabolic extracts were analyzed using a broad-spectrum platform comprising two chromatographic systems (HILIC and RPLC) and two ionization modes (positive and negative). Data were acquired on a Q Exactive HF mass spectrometer for HILIC and a Q Exactive mass spectrometer for RPLC (Thermo Scientific, San Jose, CA, USA). Both instruments were equipped with a HESI-II probe and operated in full MS scan mode. MS/MS data were acquired on quality control samples (QC) consisting of an equimolar mixture of all samples in the study. HILIC experiments were performed using a ZIC-HILIC column 2.1 × 100 mm, 3.5 µm, 200 Å (cat# 1504470001, Millipore, Burlington, MA, USA) and mobile phase solvents consisting of 10-mM ammonium acetate in 50/50 acetonitrile/water (A) and 10-mM ammonium acetate in 95/5 acetonitrile/water (B). RPLC experiments were performed using a Zorbax SBaq column 2.1 × 50 mm, 1.7 µm, 100 Å (cat# 827700-914, Agilent Technologies, Santa Clara, CA) and mobile phase solvents consisting of 0.06% acetic acid in water (A) and 0.06% acetic acid in methanol (B). Data quality was ensured by (i) injecting 6 and 12 pooled samples to equilibrate the LC-MS system prior to run the sequence for RPLC and HILIC, respectively, (ii) injecting a pool sample every 10 injections to control for signal deviation with time, and (iii) checking mass accuracy, retention time and peak shape of internal standards in each sample.

Data processing Data from each mode were independently processed using Progenesis QI software (v2.3, Nonlinear Dynamics, Durham, NC). Metabolic features from blanks and that did not show sufficient linearity upon dilution in QC samples (r < 0.6) were discarded. Only metabolic features present in >⅔ of the samples were kept for further analysis. Inter- and intra-batch variations were corrected using the LOESS (locally estimated scatterplot smoothing Local Regression) normalization method on QC injected repetitively along the batches (span = 0.75). Data were acquired in five and three batches for HILIC and RPLC modes, respectively. Missing values were imputed by drawing from a random distribution of low values in the corresponding sample. Data from each mode were merged and resulted in a dataset containing 3,529 metabolic features that was used for downstream analysis. Metabolic features of interest were tentatively identified by matching fragmentation spectra and retention time to analytical-grade standards when possible or matching experimental MS/MS to fragmentation spectra in publicly available databases. 12 of the 24 metabolomic most informative model features were successfully annotated with metabolite identifiers derived from public data bases and subsequently visualized. In individual cases, metabolite features were additionally verified by comparing their peaks to commercially available metabolite standards. Three metabolites with elemental composition C₂₁H₃₀O₃ (331.2264_8.4, 331.2264_8.1, 331.2265_8.9) were identified as isomers of 17-Hydroxyprogesterone, which correlated with 17-Hydroxyprogesterone, indicating similar biological functions and/or belonging to similar pathways. Similarly, metabolite 361.2017_7.1 (C₂₁H₃₀O₅) was highly correlated with the peak of the standard metabolite for cortisol and identified as its isomer.

Statistical analyses. Multivariate modeling and SG For a matrix X of all biological features from a given omic dataset, and a vector of days to day of labor Y, the LASSO algorithm calculates coefficients β to minimize the error term L(β) = ||Y - Xβ||². An L1 regularization was used to increase model sparsity for the sake of biological interpretation and model validation. Once a LASSO model was trained for each omics modality, the multiomic analysis was carried out by performing SG on the new representation of the data by using the outputs of the previous layer of models as predictors. A LASSO model was first constructed on each omic modality. Then, all estimations of TL were used as predictors for a second-layer LASSO model. Intrinsically, this is equivalent to a weighted average of the individual models with the coefficients of the LASSO model as desired weights. A two-layer leave-one-subject-out cross-validation strategy was used to assess the generalizability of the SG model built on the training cohort (see the Supplementary Materials). Performance for training and validation was evaluated using RMSE, and the test statistic is based on Pearson’s product moment correlation coefficient. The asymptotic confidence interval is given on the basis of Fisher’s Z transform.

Piecewise fused LASSO regression To identify a possible “switch point” before labor, we used two sequential LASSO models applied to all samples before/after a given threshold. Cross-validation predictions from both models were combined to develop a joint goodness-of-fit score for the entire dataset. The threshold was varied across the dataset to identify the point with the best fit for the combined models. Fused LASSO, a generalized LASSO for one-dimensional sequential data, which penalizes the absolute differences in successive coordinates of the LASSO coefficients, was used to detect the interval in which the joint models had the strongest predictive power, representing the region where the maximal change of biological behavior occurs before delivery.

Cross-validation An underlying assumption of the LASSO algorithm is statistical independence between all observations. In this analysis, although participants are independent, the samples collected on different days throughout the 100 days before the day of labor corresponding to the same subject are not. To address this, a leave-one-subject-out cross-validation (LOOCV) strategy was designed. In this setting, a model is trained on all available samples from all subjects but one. This procedure is repeated for each subject and a model is trained excluding it from the training. The remaining sample is used for testing. The reported results are exclusively based on the blinded subject. For stacked generalization, a two-layer cross-validation strategy was implemented where the inner layer selects the best values of λ. Then, the outer layer tests the models on the blinded subjects. A similar strategy was used for the stacked generalization step. Cross-validation folds were carefully synchronized between the individual models from each of the omics. Features whose median across all LOOCV iterations have a non-zero coefficient were reported in the set of most informative features for the prediction task. To assess the relative importance of each feature to the model, features within each individual omic data set were ranked based on the model contribution index, calculated from (-log10(p-value)*abs(model coefficient)).

Model validation Using the results for the cross-validated models on each omic as well as for the SG model combining the omics on the training cohort, we validated the results for the model by predicting the TL of each new sample of the test cohort (N=10 patients, n=27 samples). Metabolomic, proteomic and mass cytometry data for the test cohort was generated using the same procedure as for the training cohort and the features were selected by the individual LASSO to obtain the predictions. The final model coefficients are the medians of the LASSO coefficient across each fold of the cross-validation procedure. Finally, we applied the same preprocessing on the predictions obtained from each omic and computed the final SG model predictions using the same method as described above. In the metabolomic panel, one feature of the training cohort was not detected in the test cohort (331.2264_8.1). In order to compute the predictions, we simply did not include this term in the SG regression equation. Proteomic measurements were unavailable for 6 of the 27 test samples. For these 6 samples, we did not include the proteomic prediction term in the SG regression equation.

Correlation network All features in each individual omic dataset were visualized using graph structures. Each biological feature was denoted by a node. The graph was visualized using the t-SNE algorithm applied to the complete correlation matrix. For visualization purposes, only the top correlations among features were selected manually and are represented by edges.

Pattern fitting A classification method was designed to identify function patterns in the features studied. The method was first to separate features with a linear behavior from features with a quadratic behavior in relation to time to labor and then determine if the second derivative of the quadratic fits was positive (acceleration) or negative (deceleration). The first step of this classification method compared two linear regression fits for each feature Xi: one using the feature Xi and the other using the feature Xi and its square, Xi². Both fits were compared using Akaike information criterion (AIC), and the model with the lower AIC value was selected. The AIC values goodness-of-fit, but penalizes the number of parameters in the models. In this case, if the squared feature, Xi², did not sufficiently increase the goodness-of fit, the feature was considered linear. Then the feature is classified as accelerating or decelerating based on the coefficients of the model fitted. The fits chosen were associated with p-values computed from the F-statistic. The p-value (< 0.05) were used to determine the relevance of the fit chosen and discard the fits with poor association with either a linear or quadratic model.

Interactome analysis The interactome was described by the Spearman correlation coefficients between features from different omics. From the correlation matrix of all features, we filtered different thresholds to visualize the connections between the different omics. The intensity of a link between each omic was computed from this filtered correlation matrix: we counted the number of correlations passing the threshold for features from one omic with features from the other omic and we normalized by the total number of possible interactions between both -omics. In order to control for the FDR, we applied a decoy-to-target method generating one random feature using random sampling with replacement from each real feature in our multi-omic dataset. The generated “decoy” dataset of randomized features was then used to estimate the correlations passing different FDR thresholds. All correlations are controlled at FDR < 0.05.

Confounder analysis Linear regression analysis was used as a statistical model to examine the association between multiple covariates available including the model cross-validated values and the TL outcome variable. This model can be employed as a multiple linear regression to see through confounding and isolate the relationship of interest. Using this method on the training dataset we identified the confounding effects of the covariate variables and asserted the validity of the models and robustness to confounders.

Bootstrap analysis and Comparison of Ranking For each omics dataset, we performed a bootstrap analysis where we repeat a random sampling with replacement procedure on the dataset and train a cross-validated model. At each iteration, we keep the non-zero coefficients selected by the LASSO model on the bootstrapped dataset and we repeat the procedure 1000 times. We report the frequency of selection of the features as well as their median coefficient in all the bootstraps. To assess the relative importance of each feature to the model, we ranked features in each omic dataset based on their frequency of selection. This allowed us to compare the importance of the feature between the complete and term-only model to assess the robustness of the top features to the preterm samples.

Pathway enrichment analysis Pathway enrichment was performed on the top proteomics and metabolomics features using the Fisher’s test and Hypergeometric test, respectively. In a first analysis, all 45 selected features from each modality were included in the pathway analysis. To further examine the possibility of multiple correlations of interacting features across omics data contributing to different pathways, the top hits from the multivariate model were visualized using a correlation network. The nodes were divided into two major clusters and were similarly analyzed for pathway enrichment.

1. L. Liu, S. Oza, D. Hogan, Y. Chu, J. Perin, J. Zhu, J. E. Lawn, S. Cousens, C. Mathers, R. E. Black, Global, regional, and national causes of under-5 mortality in 2000-15: An updated systematic analysis with implications for the Sustainable Development Goals. Lancet 388, 3027-3035 (2016).

2. M. Galal, I. Symonds, H. Murray, F. Petraglia, R. Smith, Postterm pregnancy. Facts Views Vis. Obgyn. 4, 175-187 (2012).

3. H. M. Georgiou, M. K. W. Di Quinzio, M. Permezel, S. P. Brennecke, Predicting preterm labour: Current status and future prospects. Dis. Markers 2015, 435014 (2015).

4. N. Suff, L. Story, A. Shennan, The prediction of preterm delivery: What is new? Semin. Fetal Neonatal Med. 24, 27-32 (2019).

5. J. Hutcheon, L. Lee, G. Marquette, 320: Predicting the onset of spontaneous labour (OSL) in post date pregnancies. Am. J. Obstet. Gynecol. 208, S143-S144 (2013).

6. W. B. Barr, C. C. Pecci, Last menstrual period versus ultrasound for pregnancy dating. Int. J. Gynaecol. Obstet. 87, 38-39 (2004).

7. D. A. Savitz, J. W. Terry Jr., N. Dole, J. M. Thorp Jr., A. M. Siega-Riz, A. H. Herring, Comparison of pregnancy dating by last menstrual period, ultrasound scanning, and their combination. Am. J. Obstet. Gynecol. 187, 1660-1666 (2002).

8. Committee on Obstetric Practice American Institute of Ultrasound in Medicine Society for Maternal-Fetal Medicine, Committee Opinion No 700: Methods for estimating the due date. Obstet. Gynecol. 129, e150-e154 (2017).

9. L. S. Peterson, I. A. Stelzer, A. S. Tsai, M. S. Ghaemi, X. Han, K. Ando, V. D. Winn, N. R. Martinez, K. Contrepois, M. N. Moufarrej, S. Quake, D. A. Relman, M. P. Snyder, G. M. Shaw, D. K. Stevenson, R. J. Wong, P. Arck, M. S. Angst, N. Aghaeepour, B. Gaudilliere, Multiomic immune clockworks of pregnancy. Semin. Immunopathol. 42, 397-412 (2020).

10. M. PrabhuDas, E. Bonney, K. Caron, S. Dey, A. Erlebacher, A. Fazleabas, S. Fisher, T. Golos, M. Matzuk, J. M. McCune, G. Mor, L. Schulz, M. Soares, T. Spencer, J. Strominger, S. S. Way, K. Yoshinaga, Immune mechanisms at the maternal-fetal interface: Perspectives and challenges. Nat. Immunol. 16, 328-334 (2015).

11. P. C. Arck, K. Hecher, Fetomaternal immune cross-talk and its consequences for maternal and offspring’s health. Nat. Med. 19, 548-556 (2013).

12. N. Aghaeepour, E. A. Ganio, D. McIlwain, A. S. Tsai, M. Tingle, S. Van Gassen, D. K. Gaudilliere, Q. Baca, L. McNeil, R. Okada, M. S. Ghaemi, D. Furman, R. J. Wong, V. D. Winn, M. L. Druzin, Y. Y. El-Sayed, C. Quaintance, R. Gibbs, G. L. Darmstadt, G. M. Shaw, D. K. Stevenson, R. Tibshirani, G. P. Nolan, D. B. Lewis, M. S. Angst, B. Gaudilliere, An immune clock of human pregnancy. Sci. Immunol. 2, eaan2946 (2017).

13. T. T. M. Ngo, M. N. Moufarrej, M.-L. H. Rasmussen, J. Camunas-Soler, W. Pan, J. Okamoto, N. F. Neff, K. Liu, R. J. Wong, K. Downes, R. Tibshirani, G. M. Shaw, L. Skotte, D. K. Stevenson, J. R. Biggio, M. A. Elovitz, M. Melbye, S. R. Quake, Noninvasive blood tests for fetal development predict gestational age and preterm delivery. Science 360, 1133-1136 (2018).

14. M. S. Ghaemi, D. B. DiGiulio, K. Contrepois, B. Callahan, T. T. M. Ngo, B. Lee-McMullen, B. Lehallier, A. Robaczewska, D. Mcilwain, Y. Rosenberg-Hasson, R. J. Wong, C. Quaintance, A. Culos, N. Stanley, A. Tanada, A. Tsai, D. Gaudilliere, E. Ganio, X. Han, K. Ando, L. McNeil, M. Tingle, P. Wise, I. Maric, M. Sirota, T. Wyss-Coray, V. D. Winn, M. L. Druzin, R. Gibbs, G. L. Darmstadt, D. B. Lewis, V. P. Nia, B. Agard, R. Tibshirani, G. Nolan, M. P. Snyder, D. A. Relman, S. R. Quake, G. M. Shaw, D. K. Stevenson, M. S. Angst, B. Gaudilliere, N. Aghaeepour, Multiomics modeling of the immunome, transcriptome, microbiome, proteome and metabolome adaptations during human pregnancy. Bioinformatics 35, 95-103 (2019).

15. N. Gomez-Lopez, R. Romero, S. S. Hassan, G. Bhatti, S. M. Berry, J. P. Kusanovic, P. Pacora, A. L. Tarca, The cellular transcriptome in the maternal circulation during normal pregnancy: A longitudinal study. Front. Immunol. 10, 2863 (2019).

16. R. Romero, O. Erez, E. Maymon, P. Chaemsaithong, Z. Xu, P. Pacora, T. Chaiworapongsa, B. Done, S. S. Hassan, A. L. Tarca, The maternal plasma proteome changes as a function of gestational age in normal pregnancy: A longitudinal study. Am. J. Obstet. Gynecol. 217, 67.e1-67.e21 (2017).

17. R. Apps, Y. Kotliarov, F. Cheung, K. L. Han, J. Chen, A. Biancotto, A. Babyak, H. Zhou, R. Shi, L. Barnhart, S. M. Osgood, Y. Belkaid, S. M. Holland, J. S. Tsang, C. S. Zerbe, Multimodal immune phenotyping of maternal peripheral blood in normal human pregnancy. JCI Insight 5, e134838 (2020).

18. O. Shynlova, Y.-H. Lee, K. Srikhajon, S. J. Lye, Physiologic uterine inflammation and labor onset: Integration of endocrine and mechanical signals. Reprod. Sci. 20, 154-167 (2013).

19. N. Gomez-Lopez, D. StLouis, M. A. Lehr, E. N. Sanchez-Rodriguez, M. Arenas-Hernandez, Immune cells in term and preterm labor. Cell. Mol. Immunol. 11, 571-581 (2014).

20. C. R. Mendelson, Minireview: Fetal-maternal hormonal signaling in pregnancy and labor. Mol. Endocrinol. 23, 947-954 (2009).

21. M. McLean, A. Bisits, J. Davies, R. Woods, P. Lowry, R. Smith, A placental clock controlling the length of human pregnancy. Nat. Med. 1, 460-463 (1995).

22. R. Menon, L. S. Richardson, M. Lappas, Fetal membrane architecture, aging and inflammation in pregnancy and parturition. Placenta 79, 40-45 (2019).

23. E. R. Norwitz, J. N. Robinson, J. R. Challis, The control of labor. N. Engl. J. Med. 341, 660-666 (1999).

24. F. G. Cunningham, K. J. Leveno, S. L. Bloom, C. Y. Spong, J. S. Dashe, B. L. Hoffman, B. M. Casey, J. S. Sheffield, Williams Obstetrics (McGraw Hill, ed. 24, 2013).

25. A. L. Tarca, R. Romero, N. Benshalom-Tirosh, N. G. Than, D. W. Gudicha, B. Done, P. Pacora, T. Chaiworapongsa, B. Panaitescu, D. Tirosh, N. Gomez-Lopez, S. Draghici, S. S. Hassan, O. Erez, The prediction of early preeclampsia: Results from a longitudinal proteomics study. PLOS ONE 14, e0217273 (2019).

26. K. Contrepois, L. Jiang, M. Snyder, Optimized analytical procedures for the untargeted metabolomic profiling of human urine and plasma by combining hydrophilic interaction (HILIC) and reverse-phase liquid chromatography (RPLC)-mass spectrometry. Mol. Cell. Proteomics 14, 1684-1695 (2015).

27. L. Gold, D. Ayers, J. Bertino, C. Bock, A. Bock, E. N. Brody, J. Carter, A. B. Dalby, B. E. Eaton, T. Fitzwater, D. Flather, A. Forbes, T. Foreman, C. Fowler, B. Gawande, M. Goss, M. Gunn, S. Gupta, D. Halladay, J. Heil, J. Heilig, B. Hicke, G. Husar, N. Janjic, T. Jarvis, S. Jennings, E. Katilius, T. R. Keeney, N. Kim, T. H. Koch, S. Kraemer, L. Kroiss, N. Le, D. Levine, W. Lindsey, B. Lollo, W. Mayfield, M. Mehan, R. Mehler, S. K. Nelson, M. Nelson, D. Nieuwlandt, M. Nikrad, U. Ochsner, R. M. Ostroff, M. Otis, T. Parker, S. Pietrasiewicz, D. I. Resnicow, J. Rohloff, G. Sanders, S. Sattin, D. Schneider, B. Singer, M. Stanton, A. Sterkel, A. Stewart, S. Stratford, J. D. Vaught, M. Vrkljan, J. J. Walker, M. Watrobka, S. Waugh, A. Weiss, S. K. Wilcox, A. Wolfson, S. K. Wolk, C. Zhang, D. Zichi, Aptamerbased multiplexed proteomic technology for biomarker discovery. PLOS ONE 5, e15004 (2010).

28. D. H. Wolpert, Stacked generalization. Neural Netw. 5, 241-259 (1992).

29. I. Rivals, L. Personnaz, L. Taing, M.-C. Potier, Enrichment or depletion of a GO category within a class of genes: Which test? Bioinformatics 23, 401-407 (2007).

30. J. Xia, I. V. Sinelnikov, B. Han, D. S. Wishart, MetaboAnalyst 3.0-Making metabolomics more meaningful. Nucleic Acids Res. 43, W251-W257 (2015).

31. B. R. Carr, C. R. Parker Jr., J. D. Madden, P. C. MacDonald, J. C. Porter, Maternal plasma adrenocorticotropin and cortisol relationships throughout human pregnancy. Am. J. Obstet. Gynecol. 139, 416-422 (1981).

32. R. Tai, H. S. Taylor, Endocrinology of Pregnancy (MDText.com Inc., 2000).

33. H. Singh, J. D. Aplin, Endometrial apical glycoproteomic analysis reveals roles for cadherin 6, desmoglein-2 and plexin b2 in epithelial integrity. Mol. Hum. Reprod. 21, 81-94 (2015).

34. W. F. Vogel, A. Asz.di, F. Alves, T. Pawson, Discoidin domain receptor 1 tyrosine kinase has an essential role in mammary gland development. Mol. Cell. Biol. 21, 2906-2917 (2001).

35. E. Geva, D. G. Ginzinger, C. J. Zaloudek, D. H. Moore, A. Byrne, R. B. Jaffe, Human placental vascular development: Vasculogenic and angiogenic (branching and nonbranching) transformation is regulated by vascular endothelial growth factor-A, angiopoietin-1, and angiopoietin-2. J. Clin. Endocrinol. Metab. 87, 4213-4224 (2002).

36. M. Bolin, E. Wiberg-Itzel, A.-K. Wikstr.m, M. Goop, A. Larsson, M. Olovsson, H. Akerud, Angiopoietin-⅟angiopoietin-2 ratio for prediction of preeclampsia. Am. J. Hypertens. 22, 891-895 (2009).

37. D. Tulchinsky, H. H. Simmer, Sources of plasma 17 α-hydroxyprogesterone in human pregnancy. J. Clin. Endocrinol. Metab. 35, 799-808 (1972).

38. M. Abbassi-Ghanavati, L. G. Greer, F. G. Cunningham, Pregnancy and laboratory studies: A reference table for clinicians. Obstet. Gynecol. 114, 1326-1331 (2009).

39. K. D. Pennell, M. A. Woodin, P. B. Pennell, Quantification of neurosteroids during pregnancy using selective ion monitoring mass spectrometry. Steroids 95, 24-31 (2015).

40. I. Granne, J. H. Southcombe, J. V. Snider, D. S. Tannetta, T. Child, C. W. G. Redman, I. L. Sargent, ST2 and IL-33 in pregnancy and pre-eclampsia. PLOS ONE 6, e24463 (2011).

41. R. Romero, P. Chaemsaithong, A. L. Tarca, S. J. Korzeniewski, E. Maymon, P. Pacora, B. Panaitescu, N. Chaiyasit, Z. Dong, O. Erez, S. S. Hassan, T. Chaiworapongsa, Maternal plasma-soluble ST2 concentrations are elevated prior to the development of early and late onset preeclampsia - a longitudinal study. J. Matern. Fetal Neonatal Med. 31, 418-432 (2018).

42. F. Y. Liew, J.-P. Girard, H. R. Turnquist, Interleukin-33 in health and disease. Nat. Rev. Immunol. 16, 676-689 (2016).

43. B. Huang, A. N. Faucette, M. D. Pawlitz, B. Pei, J. W. Goyert, J. Z. Zhou, N. G. El-Hage, J. Deng, J. Lin, F. Yao, R. S. Dewar III, J. S. Jassal, M. L. Sandberg, J. Dai, M. Cols, C. Shen, L. A. Polin, R. A. Nichols, T. B. Jones, M. H. Bluth, K. S. Puder, B. Gonik, N. R. Nayak, E. Puscheck, W. Z. Wei, A. Cerutti, M. Colonna, K. Chen, Interleukin-33-induced expression of PIBF1 by decidual B cells protects against preterm labor. Nat. Med. 23, 128-135 (2017).

44. A. H. James, E. Rhee, B. Thames, C. S. Philipp, Characterization of antithrombin levels in pregnancy. Thromb. Res. 134, 648-651 (2014).

45. C. S. Buhimschi, V. Bhandari, A. T. Dulay, S. Thung, S. S. Abdel- Razeq, V. Rosenberg, C. S. Han, U. A. Ali, E. Zambrano, G. Zhao, E. F. Funai, l. A. Buhimschi, Amniotic fluid angiopoietin-1, angiopoietin-2, and soluble receptor tunica interna endothelial cell kinase-2 levels and regulation in normal pregnancy and intraamniotic inflammation induced preterm birth. J. Clin. Endocrinol. Metab. 95, 3428-3436 (2010).

46. N. Gomez-Lopez, L. Vadillo-Perez, S. Nessim, D. M. Olson, F. Vadillo-Ortega, Choriodecidua and amnion exhibit selective leukocyte chemotaxis during term human labor. Am. J. Obstet. Gynecol. 204, 364.e9-364.e16 (2011).

47. C. Seiler, N. L. Bayless, R. Vergara, J. Pintye, J. Kinuthia, L. Osborn, D. Matemo, B. A. Richardson, G. John-Stewart, S. Holmes, C. A. Blish, Influenza-induced interferon lambda response is associated with longer time to delivery among pregnant Kenyan women. Front. Immunol. 11, 452 (2020).

48. X. Han, M. S. Ghaemi, K. Ando, L. S. Peterson, E. A. Ganio, A. S. Tsai, D. K. Gaudilliere, l. A. Stelzer, J. Einhaus, B. Bertrand, N. Stanley, A. Culos, A. Tanada, J. Hedou, E. S. Tsai, R. Fallahzadeh, R. J. Wong, A. E. Judy, V. D. Winn, M. L. Druzin, Y. J. Blumenfeld, M. A. Hlatky, C. C. Quaintance, R. S. Gibbs, B. Carvalho, G. M. Shaw, D. K. Stevenson, M. S. Angst, N. Aghaeepour, B. Gaudilliere, Differential dynamics of the maternal immune system in healthy pregnancy and preeclampsia. Front. Immunol. 10, 1305 (2019).

49. J. Ghartey, L. Anglim, J. Romero, A. Brown, M. A. Elovitz, Women with symptomatic preterm birth have a distinct cervicovaginal metabolome. Am. J. Perinatol. 34, 1078-1083 (2017).

50. J. M. Fettweis, M. G. Serrano, J. P. Brooks, D. J. Edwards, P. H. Girerd, H. l. Parikh, B. Huang, T. J. Arodz, L. Edupuganti, A. L. Glascock, J. Xu, N. R. Jimenez, S. C. Vivadelli, S. S. Fong, N. U. Sheth, S. Jean, V. Lee, Y. A. Bokhari, A. M. Lara, S. D. Mistry, R. A. Duckworth III, S. P. Bradley, V. N. Koparde, X. V. Orenda, S. H. Milton, S. K. Rozycki, A. V. Matveyev, M. L. Wright, S. V. Huzurbazar, E. M. Jackson, E. Smirnova, J. Korlach, Y.-C. Tsai, M. R. Dickinson, J. L. Brooks, J. l. Drake, D. O. Chaffin, A. L. Sexton, M. G. Gravett, C. E. Rubens, N. R. Wijesooriya, K. D. Hendricks-Mu.oz, K. K. Jefferson, J. F. Strauss III, G. A. Buck, The vaginal microbiome and preterm birth. Nat. Med. 25, 1012-1021 (2019).

51. E. Amabebe, D. R. Chapman, V. L. Stern, G. Stafford, D. O. C. Anumba, Mid-gestational changes in cervicovaginal fluid cytokine levels in asymptomatic pregnant women are predictive markers of inflammation-associated spontaneous preterm birth. J. Reprod. Immunol. 126, 1-10 (2018).

52. l. Kosti, S. Lyalina, K. S. Pollard, A. J. Butte, M. Sirota, Meta-analysis of vaginal microbiome data provides new insights into preterm birth. Front. Microbiol. 11, 476 (2020).

53. G. R. Saade, K. A. Boggess, S. A. Sullivan, G. R. Markenson, J. D. lams, D. V. Coonrod, L. M. Pereira, M. S. Esplin, L. M. Cousins, G. K. Lam, M. K. Hoffman, R. D. Severinsen, T. Pugmire, J. S. Flick, A. C. Fox, A. J. Lueth, S. R. Rust, E. Mazzola, C. Hsu, M. T. Dufford, C. L. Bradford, l. E. Ichetovkin, T. C. Fleischer, A. D. Polpitiya, G. C. Critchfield, P. E. Kearney, J. J. Boniface, D. E. Hickok, Development and validation of a spontaneous preterm delivery predictor in asymptomatic women. Am. J. Obstet. Gynecol. 214, 633.e1-633.e24 (2016).

54. N. M. Shah, P. F. Lai, N. Imami, M. R. Johnson, Progesterone-related immune modulation of pregnancy and labor. Front. Endocrinol. (Lausanne) 10, 198 (2019).

55. S. Mesiano, Myometrial progesterone responsiveness. Semin. Reprod. Med. 25, 005-013 (2007).

56. N. M. Shah, A. A. Herasimtschuk, A. Boasso, A. Benlahrech, D. Fuchs, N. Imami, M. R. Johnson, Changes in T cell and dendritic cell phenotype from mid to late pregnancy are indicative of a shift from immune tolerance to immune activation. Front. Immunol. 8, 1138 (2017).

57. P. Luppi, C. Haluszczak, D. Betters, C. A. H. Richard, M. Trucco, J. A. DeLoia, Monocytes are progressively activated in the circulation of pregnant women. J. Leukoc. Biol. 72, 874-884 (2002).

58. K. Taniguchi, H. Nagata, T. Katsuki, C. Nakashima, R. Onodera, A. Hiraoka, N. Takata, M. Kobayashi, M. Kambe, Significance of human neutrophil antigen-2a (NB1) expression and neutrophil number in pregnancy. Transfusion 44, 581-585 (2004).

59. S. Lurie, E. Rahamim, l. Piper, A. Golan, O. Sadan, Total and differential leukocyte counts percentiles in normal pregnancy. Eur. J. Obstet. Gynecol. Reprod. Biol. 136, 16-19 (2008).

60. S. M. Ziegler, C. N. Feldmann, S. H. Hagen, L. Richert, T. Barkhausen, J. Goletzke, V. Jazbutyte, G. Martrus, W. Salzberger, T. Renn., K. Hecher, A. Diemert, P. C. Arck, M. Altfeld, Innate immune responses to toll-like receptor stimulation are altered during the course of pregnancy. J. Reprod. Immunol. 128, 30-37 (2018).

61. A. L. Tarca, R. Romero, Z. Xu, N. Gomez-Lopez, O. Erez, C.-D. Hsu, S. S. Hassan, V. J. Carey, Targeted expression profiling by RNA-Seq improves detection of cellular dynamics during pregnancy and identifies a role for T cells in term parturition. Sci. Rep. 9, 848 (2019).

62. R. Pique-Regi, R. Romero, A. L. Tarca, E. D. Sendler, Y. Xu, V. Garcia-Flores, Y. Leng, F. Luca, S. S. Hassan, N. Gomez-Lopez, Single cell transcriptional signatures of the human placenta in term and preterm parturition. eLife 8, e52004 (2019).

63. R. Obrenovic, D. Petrovic, N. Majkic-Singh, J. Trbojevic-Stankovic, B. Stojimirovic, Serum cystatin C levels in normal pregnancy. Clin. Nephrol. 76, 174-179 (2011).

64. R. Menon, Initiation of human parturition: Signaling from senescent fetal tissues via extracellular vesicle mediated paracrine mechanism. Obstet. Gynecol. Sci. 62, 199-211 (2019).

65. J. Polettini, F. Behnia, B. D. Taylor, G. R. Saade, R. N. Taylor, R. Menon, Telomere fragment induced amnion cell senescence: A contributor to parturition? PLOS ONE 10, e0137188 (2015).

66. M. D. Mitchell, H. N. Peiris, M. Kobayashi, Y. Q. Koh, G. Duncombe, S. E. Illanes, G. E. Rice, C. Salomon, Placental exosomes in normal and complicated pregnancy. Am. J. Obstet. Gynecol. 213, S173-S181 (2015).

67. Y. M. D. Lo, N. Corbetta, P. F. Chamberlain, V. Rai, l. L. Sargent, C. W. G. Redman, J. S. Wainscoat, Presence of fetal DNA in maternal plasma and serum. Lancet 350, 485-487 (1997).

68. J. M. Kinder, l. A. Stelzer, P. C. Arck, S. S. Way, Immunological implications of pregnancy induced microchimerism. Nat. Rev. Immunol. 17, 483-494 (2017).

69. R. Menon, F. Behnia, J. Polettini, G. R. Saade, J. Campisi, M. Velarde, Placental membrane aging and HMGB1 signaling associated with human parturition. Aging (Albany NY) 8, 216-230 (2016).

70. F. Gotsch, R. Romero, J. P. Kusanovic, O. Erez, J. Espinoza, C. J. Kim, E. Vaisbuch, N. G. Than, S. Mazaki-Tovi, T. Chaiworapongsa, M. Mazor, B. H. Yoon, S. Edwin, R. Gomez, P. Mittal, S. S. Hassan, S. Sharma, The anti-inflammatory limb of the immune response in preterm labor, intra-amniotic infection/inflammation, and spontaneous parturition at term: A role for interleukin-10. J. Matern. Fetal Neonatal Med. 21, 529-547 (2008).

71. T. A. Kraus, S. M. Engel, R. S. Sperling, L. Kellerman, Y. Lo, S. Wallenstein, M. M. Escribese, J. L. Garrido, T. Singh, M. Loubeau, T. M. Moran, Characterizing the pregnancy immune phenotype: Results of the viral immunity and pregnancy (VIP) study. J. Clin. Immunol. 32, 300-311 (2012).

72. l. P. Crocker, P. N. Baker, J. Fletcher, Neutrophil function in pregnancy and rheumatoid arthritis. Ann. Rheum. Dis. 59, 555-564 (2000).

73. J. M. Cha, D. M. Aronoff, A role for cellular senescence in birth timing. Cell Cycle 16, 2023-2031 (2017).

74. L. Yu, D. Li, Q.-p. Liao, H.-x. Yang, B. Cao, G. Fu, G. Ye, Y. Bai, H. Wang, N. Cui, M. Liu, Y.-x. Li, J. Li, C. Peng, Y.-I. Wang, High levels of activin A detected in preeclamptic placenta induce trophoblast cell apoptosis by promoting nodal signaling. J. Clin. Endocrinol. Metab. 97, E1370-E1379 (2012).

75. S. Muttukrishna, P. A. Fowler, L. George, N. P. Groome, P. G. Knight, Changes in peripheral serum levels of total activin A during the human menstrual cycle and pregnancy. J. Clin. Endocrinol. Metab. 81, 3328-3334 (1996).

76. M. P. Plevyak, G. M. Lambert-Messerlian, A. Farina, N. P. Groome, J. A. Canick, H. M. Silver, Concentrations of serum total activin A and inhibin A in preterm and term labor patients: A cross-sectional study. J. Soc. Gynecol. Investig. 10, 231-236 (2003).

77. K. K. Rumer, J. Uyenishi, M. C. Hoffman, B. M. Fisher, V. D. Winn, Siglec-6 expression is increased in placentas from pregnancies complicated by preterm preeclampsia. Reprod. Sci. 20, 646-653 (2013).

78. E. C. M. Brinkman-Van der Linden, N. Hurtado-Ziola, T. Hayakawa, L. Wiggleton, K. Benirschke, A. Varki, N. Varki, Human-specific expression of Siglec-6 in the placenta. Glycobiology 17, 922-931 (2007).

79. N. Aghaeepour, B. Lehallier, Q. Baca, E. A. Ganio, R. J. Wong, M. S. Ghaemi, A. Culos, Y. Y. El-Sayed, Y. J. Blumenfeld, M. L. Druzin, V. D. Winn, R. S. Gibbs, R. Tibshirani, G. M. Shaw, D. K. Stevenson, B. Gaudilliere, M. S. Angst, A proteomic clock of human pregnancy. Am. J. Obstet. Gynecol. 218, 347.e1-347.e14 (2018).

80. M. E. Sowa, E. J. Bennett, S. P. Gygi, J. W. Harper, Defining the human deubiquitinating enzyme interaction landscape. Cell 138, 389-403 (2009).

81. S. M. Blois, G. Sulkowski, l. Tirado-Gonz.lez, J. Warren, N. Freitag, B. F. Klapp, D. Rifkin, I. Fuss, W. Strober, G. S. Dveksler, Pregnancy-specific glycoprotein 1 (PSG1) activates TGF-β and prevents dextran sodium sulfate (DSS)-induced colitis in mice. Mucosal Immunol. 7, 348-358 (2014).

82. N. Carretti, A. Bertazzo, S. Comai, C. V. L. Costa, G. Allegri, F. Petraglia, Serum tryptophan and 5-hydroxytryptophan at birth and during post-partum days. Adv. Exp. Med. Biol. 527, 757-760 (2003).

83. M. A. Cruz, V. Gallardo, P. Miguel, G. Carrasco, C. Gonz.lez, Serotonin-induced vasoconstriction is mediated by thromboxane release and action in the human fetal-placental circulation. Placenta 18, 197-204 (1997).

84. L. Liang, M.-L. H. Rasmussen, B. Piening, X. Shen, S. Chen, H. R.st, J. K. Snyder, R. Tibshirani, L. Skotte, N. C. Y. Lee, K. Contrepois, B. Feenstra, H. Zackriah, M. Snyder, M. Melbye, Metabolic dynamics and prediction of gestational age and time to delivery in pregnant women. Cell 181, 1680-1692.e15 (2020).

85. B. M. Mercer, E. K. S. Chien, in Gabbe’s Obstetrics: Normal and Problem Pregnancies (Elsevier, ed. 8, 2021), chap. 37, pp. 694-707.e3.

86. A. Culos, A. S. Tsai, N. Stanley, M. Becker, M. S. Ghaemi, D. R. Mcllwain, R. Fallahzadeh, A. Tanada, H. Nassar, C. Espinosa, M. Xenochristou, E. Ganio, L. Peterson, X. Han, l. A. Stelzer, K. Ando, D. Gaudilliere, T. Phongpreecha, l. Marić, A. L. Chang, G. M. Shaw, D. K. Stevenson, S. Bendall, K. L. Davis, W. Fantl, G. P. Nolan, T. Hastie, R. Tibshirani, M. S. Angst, B. Gaudilliere, N. Aghaeepour, Integration of mechanistic immunological knowledge into a machine learning pipeline improves predictions. Nat. Mach. Intell. 2, 619-628 (2020).

87. S. Aminikhanghahi, D. J. Cook, A survey of methods for time series change point detection. Knowl. Inf. Syst. 51, 339-367 (2017).

88. R. Tibshirani, M. Saunders, S. Rosset, J. Zhu, K. Knight, Sparsity and smoothness via the fused lasso. J. R. Stat. Soc. B 67, 91-108 (2005).

89. R. Finck, E. F. Simonds, A. Jager, S. Krishnaswamy, K. Sachs, W. Fantl, D. Pe’er, G. P. Nolan, S. C. Bendall, Normalization of mass cytometry data with bead standards. Cytometry 83A, 483-494 (2013).

90. E. R. Zunder, R. Finck, G. K. Behbehani, E. D. Amir, S. Krishnaswamy, V. D. Gonzalez, C. G. Lorang, Z. Bjornson, M. H. Spitzer, B. Bodenmiller, W. J. Fantl, D. Pe’er, G. P. Nolan, Palladium-based mass tag cell barcoding with a doublet-filtering scheme and single-cell deconvolution algorithm. Nat. Protoc. 10, 316-333 (2015).

91. J. C. Rohloff, A. D. Gelinas, T. C. Jarvis, U. A. Ochsner, D. J. Schneider, L. Gold, N. Janjic, Nucleic acid ligands with protein-like side chains: Modified aptamers and their use as diagnostic and therapeutic agents. Mol. Ther. Nucleic Acids 3, e201 (2014).

TABLE 1

Pregnancy cohort demographics

| Pregnancy Characteristics | Training Cohort | | Test Cohort | |
|---|---|---|---|---|
| | N = 53 n = 150 | Percentage or Median (Interquartile Range) | N= 10 n = 27 | Percentage or Median (Interquartile Range) |
| Age (years) | 53 | 33 (30, 35) | 10 | 31 (29, 33) |
| BMI 1^{st} trimester (kq/m²) | 46 | 23.4 (21.1, 25.6) | 6 | 24.3 (20.7, 25.3) |
| BMI 3^{rd} trimester (kq/m²) | 46 | 27.6 (24.8, 30.1), | 10 | 26.3 (23.5, 27.7) |
| GA at delivery, all (wks) | 53 | 39.4 (38.8, 40.0) | 10 | 39.3 (38.7, 40.3) |
| Preterm delivery (<37 wks) | 5 | 9.4% | 0 | 0% |
| Preterm GA at delivery (wks) | 5 | 34.3 (34.0, 35.3) | 0 | 0% |
| Gravidity | 53 | 2.0 (1.0, 3.0) | 10 | 1.7 (1.0, 2.0) |
| Parity (% nulliparous) | 30 | 56.6% | 6 | 60% |
| Infant sex | 23 | female (43.4%) | 2 | female (20%) |
| Birthweight (g) | 52 | 3238 (2983, 3685) | 10 | 3483 (3155, 3880) |
| 5-min Apqar score | 52 | 9 (9, 9) | 9 | 8.8 (9, 9) |
| Race | | | | |
| East Asian | 16 | 30.2% | 6 | 60% |
| South Asian | 7 | 13.2% | 0 | 0% |
| South East Asian | 3 | 5.7% | 0 | 0% |
| African-American | 0 | 0% | 0 | 0% |
| Native Hawaiian/Pacific Islander | 1 | 1.9% | 0 | 0% |
| Middle Eastern | 1 | 1.9% | 0 | 0% |
| White | 19 | 35.9% | 3 | 30% |
| Two or more races | 2 | 3.8% | 0 | 0% |
| Other | 4 | 7.6% | 1 | 10% |
| Ethnicity | | | | |
| Hispanic | 2 | 3.8% | 0 | 0% |
| Non-Hispanic | 51 | 96.2% | 10 | 100% |
| Mode of delivery | | | | |
| Spontaneous rupture of membranes | 53 | 100% | 10 | 100% |
| Spontaneous onset of labor | 53 | 100% | 5 | 50% |
| Vaginal | 48 | 90.6% | 7 | 70% |
| Cesarean section during labor | 5 | 9.4% | 3 | 30% |
| Comorbidity | | | | |
| Gestational diabetes | 4 | 7.6% | 0 | 0% |
| Polycystic ovary syndrome | 1 | 1.9% | 0 | 0% |
| Mild gestational hypertension | 4 | 7.6% | 0 | 0% |
| Mild preeclampsia | 3 | 5.7% | 0 | 0% |
| History of preterm birth | 6 | 11.3% | 0 | 0% |
| Progesterone treatment | 3 | 5.7% | 0 | 0% |

TABLE 2

Mass cytometry antibody panel

| Antibody | Manufacturer | Atomic Symbol | Atomic Mass | Clone | Comment | Catalogue Number | RRID |
|---|---|---|---|---|---|---|---|
| Barcode 1 | Trace Sciences | Pd | 102 | | Barcode | | |
| Barcode 2 | Trace Sciences | Pd | 104 | | Barcode | | |
| Barcode 3 | Trace Sciences | Pd | 105 | | Barcode | | |
| Barcode 4 | Trace Sciences | Pd | 106 | | Barcode | | |
| Barcode 5 | Trace Sciences | Pd | 108 | | Barcode | | |
| Barcode 6 | Trace Sciences | Pd | 110 | | Barcode | | |
| CD235 | BioLegend | In | 113 | HIR2 | Phenotype | 306615 | AB 2562825 |
| CD61 | BD | In | 113 | VI-PL2 | Phenotype | 555752 | AB_396093 |
| CD45 | Biolegend | In | 115 | HI30 | Phenotype | 304045 | AB_2562821 |
| CD66 | BD | La | 139 | B1.1/CD66 | Phenotype | 551354 | AB_394166 |
| CD7 | BD | Pr | 141 | M-T701 | Phenotype | 555359 | AB_395762 |
| CD19 | BioLegend | Nd | 142 | HIB19 | Phenotype | 302247 | AB_2562815 |
| CD45RA | BioLegend | Nd | 143 | HI100 | Phenotype | 304143 | AB_2562822 |
| CD11b | BioLegend | Nd | 144 | ICRF44 | Phenotype | 301337 | AB_2562811 |
| CD4 | BioLegend | Nd | 145 | RPA-T4 | Phenotype | 300541 | AB_2562809 |
| CD8a | BioLegend | Nd | 146 | RPA-T8 | Phenotype | 301053 | AB-256281 0 |
| CD11c | BioLegend | Sm | 147 | Bu15 | Phenotype | 337221 | AB_2562834 |
| CD123 | BioLegend | Nd | 148 | 6H6 | Phenotype | 306027 | AB_2562823 |
| pCREB (pS133) | CST | Sm | 149 | 87G3 | Function | 9198 | AB_2561044 |
| pSTAT5 (Tyr694) | CST | Nd | 150 | C11C5 | Function | 9359 | AB_823649 |
| pP38 (pT180/pY182) | BD | Eu | 151 | 36/p38 | Function | 612289 | AB_399606 |
| TCRγδ | BD | Sm | 152 | B1 | Phenotype | 555715 | AB_396059 |
| pSTAT1 (pY701) | BD | Eu | 153 | 14/P-STAT1 | Function | 612133 | AB_399504 |
| pSTAT3 (Tyr705) | CST | Sm | 154 | M9C6 | Function | 4113 | AB_2198588 |
| pS6 (pS235/pS236) | CST | Gd | 155 | D57.2.2E | Function | 4858 | AB_916156 |
| IκB | CST | Gd | 156 | L35A5 | Function | 4814S | |
| CD69 | BD | Gd | 157 | FN50 | Phenotype | 555529 | AB_395914 |
| CD33 | BioLegend | Gd | 158 | WM53 | Phenotype | 303419 | AB_2562818 |
| pMAPKAPK2 (Thr334) | CST | Tb | 159 | 27B7 | Function | 3007 | AB_490936 |
| Tbet | Thermo Fisher | Gd | 160 | 4B10 | Phenotype | 14-5825-80 | AB_763635 |
| cPARP | BD | Dy | 161 | F21-852 | Function | 552596 | AB_394437 |
| FoxP3 | Thermo Fisher | Dy | 162 | PCH101 | Phenotype | 14-4776-82 | AB_467554 |
| CD45RO | Fluidigm | Dy | 164 | UCHL1 | Phenotype | 3164007B | AB_2811092 |
| CD16 | BioLegend | Ho | 165 | 3G8 | Phenotype | 302051 | AB_2562814 |
| pNFκB (pS529) | BD | Er | 166 | K10-895.12.50 | Function | 558393 | AB_647284 |
| pERK1/2 (pT202/pY204) | CST | Yb | 167 | D13.14.4E | Function | 4370 | AB_2315112 |
| pSTAT6 (Tyr641) | BioLegend | Er | 168 | A15137E | Function | 686002 | AB_2616820 |
| CD25 | BioLegend | Tm | 169 | M-A251 | Phenotype | 356102 | AB_2561752 |
| CD3 | BioLegend | Er | 170 | UCHT1 | Phenotype | 300402 | AB_314056 |
| CCR5 | Fluidigm | Yb | 171 | NP6G4 | Phenotype | 3171017A | |
| CD62L | Thermo Scientific | Yb | 172 | DREG.200 | Phenotype | BMS1015 | AB_10596353 |
| CCR2 | BioLegend | Yb | 173 | K036C2 | Phenotype | 357202 | AB_2561851 |
| HLA-DR | BioLegend | Yb | 174 | L243 | Phenotype | 307651 | AB_2562826 |
| CD14 | BioLegend | Yb | 175 | M5E2 | Phenotype | 301843 | AB_2562813 |
| CD56 | BD | Yb | 176 | NCAM16.2 | Phenotype | 559043 | AB_397180 |
| DNA1 | Fluidigm | Ir | 191 | | DNA | | |
| DNA2 | Fluidigm | Ir | 192 | | DNA | | |

Confounder analysis. None of the potentially confounding variables significantly influences the prediction of the TL in the original training model (see Methods). Related to FIG. 3. *“Betamethasone treatment” is co-linear with variable “Preterm delivery (<37 wks)

TABLE 3

| Variable | Estimate | Std.Error | t | value | Pr(>ltl) |
|---|---|---|---|---|---|
| Prediction | 1.308304 | 0.059853 | 21.859 | <2e-16 | *** |
| Age (years) | 0.44338 | 0.510009 | 0.869 | 0.3867 | |
| BMI 1st trimester (kg/m2) | 2.056674 | 1.194251 | 1.722 | 0.0881 | |
| BMI 3rd trimester (kg/m2) | -0.406995 | 1.004374 | -0.405 | 0.6862 | |
| GA at delivery, all (wks) | -2.477985 | 2.186258 | -1.133 | 0.2597 | |
| Preterm delivery (<37 wks)* | -0.056306 | 11.72105 | -0.005 | 0.9962 | |
| Gravidity | -2.657588 | 1.82363 | -1.457 | 0.1481 | |
| Parity (% nulliparous) | 0.762594 | 3.100135 | 0.246 | 0.8062 | |
| Infant sex | -4.016345 | 3.494093 | -1.149 | 0.2531 | |
| Birthweight (g) | -0.00641 | 0.004302 | -1.49 | 0.1393 | |
| Race East Asian | 88.571079 | 89.228857 | 0.993 | 0.3232 | |
| Race South Asian | 81.119883 | 91.381792 | 0.888 | 0.3768 | |
| Race Southeast Asian | 87.743665 | 89.408413 | 0.981 | 0.3287 | |
| Race Native Hawaiian/Pacific Islander | 76.950741 | 92.525918 | 0.832 | 0.4075 | |
| Race Middle Eastern | 68.949986 | 90.949048 | 0.758 | 0.4501 | |
| Race White | 85.241423 | 90.572585 | 0.941 | 0.3489 | |
| Race Two or More Races | 130.86968 | 87.843884 | 1.49 | 0.1394 | |
| Race Other | 87.38296 | 92.766224 | 0.942 | 0.3484 | |
| Gestational diabetes | 3.709263 | 6.795246 | 0.546 | 0.5864 | |
| Gestational hypertension | -7.140725 | 5.651122 | -1.264 | 0.2093 | |
| History of preterm birth | 2.650519 | 6.589623 | 0.402 | 0.6884 | |
| Preeclampsia | -0.09008 | 7.124061 | -0.013 | 0.9899 | |
| Progesterone treatment | -6.987755 | 9.992922 | -0.699 | 0.486 | |

Forty-five most informative features of the integrated multiomic labor prediction model in the training (blue) and test (gray) cohort. Features in the training cohort were ranked based on the model index (calculated from: -log10(pval)*abs(model coef)). Spearman correlation coefficients and associated p-values were calculated for the association of the individual features with TL in both cohorts. Related to FIGS. 3, 4.

TABLE 4

| Modality | Feature name | Model index | Training Spearman Correlation Coefficient (R) | Training p-value | Test Spearman Correlation Coefficient (R) | Test p-value (N = 10 patients) |
|---|---|---|---|---|---|---|
| metabolome | 331.2264_8.4 (17-OHP/P4 derivative) | 200.12 | 0.82 | 3.94E-37 | 0.79 | 9.19E-07 |
| metabolome | 331.2264_8.1 (17-OHP/P4 derivative) | 161.21 | 0.76 | 1.47E-29 | Not detected | na |
| metabolome | 331.2265_8.9 (17-OHP/P4 derivative) | 47.61 | 0.69 | 5.98E-23 | 0.53 | 4.74E-03 |
| metabolome | 361.2017_7.1 (Cortisol) | 27.84 | 0.62 | 5.29E-17 | 0.39 | 4.35E-02 |
| metabolome | 415.3204_12 (C₂₇H₄₂O₃) | 16.43 | 0.63 | 8.63E-18 | 0.41 | 2.05E-03 |
| metabolome | 151.0615_2.6 (1-Methylhypoxanthine) | 8.71 | 0.53 | 3.95E-12 | 0.45 | 3.42E-02 |
| metabolome | 411.1844_8.7 (17-OH pregnenolone sulfate) | 7.73 | 0.75 | 2.10E-28 | 0.57 | 1.79E-02 |
| metabolome | 193.0618_5.3 (4-Aminohippuric acid) | 5.11 | 0.52 | 1.40E-11 | 0.21 | 3.02E-01 |
| metabolome | 151.0612_6 (Arabitol, Xylitol) | 3.01 | 0.34 | 2.69E-05 | 0.62 | 2.29E-01 |
| metabolome | 219.0774_6.3 (5-Hydroxytryptophan) | 2.42 | -0.33 | 3.57E-05 | -0.24 | 1.67E-01 |
| metabolome | 236.0929_4.3 (N-Lactoylphenylalanine) | 2.17 | 0.43 | 4.96E-08 | 0.27 | 9.52E-01 |
| metabolome | 397.205_10.6 (6 (Pregnanolone sulfate) | 0.24 | -0.15 | 7.39E-02 | -0.012 | 5.89E-04 |
| proteome | IL-1R4 | 191.68 | 0.78 | 7.98E-32 | 0.83 | 3.12E-03 |
| proteome | Plexin-B2 (PLXB2) | 89.89 | 0.71 | 3.68E-24 | 0.12 | 5.37E-01 |
| proteome | Discoidin domain receptor 1 (DDR1) | 68.74 | 0.68 | 2.84E-21 | 0.44 | 4.87E-01 |
| proteome | Angiopoietin-2 | 52.63 | -0.58 | 1.16E-14 | -0.61 | 6.13E-02 |
| proteome | Vascular Endothelial Growth Factor 121 | 45.08 | -0.56 | 1.39E-13 | -0.55 | 1.43E-01 |
| proteome | Cystatin C | 38.77 | 0.67 | 3.98E-21 | 0.26 | 5.55E-01 |
| proteome | SLIT and NTRK-like protein 5 (SLTRK5) | 31.36 | -0.49 | 2.83E-10 | -0.26 | 5.37E-01 |
| proteome | Secr. Leukocyte Peptidase Inhibitor (SLPI) | 24.90 | 0.47 | 1.73E-09 | 0.31 | 2.27E-01 |
| proteome | Activin A | 19.23 | 0.71 | 2.89E-24 | 0.34 | 9.15E-01 |
| proteome | Antithrombin III | 10.27 | -0.45 | 6.11E-09 | -0.46 | 7.33E-02 |
| proteome | Macrophage inhibitory cytokine-1 (MIC-1) | 9.96 | 0.61 | 1.79E-16 | 0.62 | 1.15E-02 |
| proteome | Siglec-6 | 8.60 | 0.73 | 4.43E-26 | 0.85 | 4.49E-03 |
| proteome | urokinase-type Plasminogen Activator (uPA) | 8.09 | 0.59 | 2.44E-15 | 0.39 | 2.42E-01 |
| proteome | Matrix Metalloproteinase (MMP) 12 | 5.87 | -0.51 | 2.67E-11 | -0.69 | 9.25E-03 |
| proteome | Soluble tunica interna endothelial cell kinase (sTie)-2 | 4.91 | -0.36 | 8.33E-06 | -0.49 | 6.80E-02 |
| proteome | LAG3 | 3.24 | 0.39 | 6.59E-07 | 0.09 | 4.68E-01 |
| proteome | Endostatin | 1.73 | 0.24 | 3.22E-03 | 0.05 | 8.43E-01 |
| proteome | GA733-1 protein | 0.27 | -0.12 | 1.52E-01 | 0.19 | 9.38E-01 |
| immunome | CD69⁻CD56^{\|o}CD16⁺NK, pSTAT1, IFNα | 161.23 | 0.61 | 1.31E-16 | 0.78 | 1.56E-06 |
| immunome | Granulocytes (freq) | 18.20 | -0.34 | 1.74E-05 | -0.31 | 1.12E-01 |
| immunome | CD69⁺CD56^{\|o}CD 16⁺NK, pSTAT1, IFNα | 10.18 | 0.58 | 5.96E-15 | 0.61 | 7.07E-04 |
| immunome | CD62L⁺CD4Tₙₐᵢᵥₑ, pMAPKAPK2, IFNα | 8.92 | 0.35 | 1.39E-05 | 0.42 | 3.01E-02 |
| immunome | ncMC, pCREB, GM-CSF | 8.11 | -0.28 | 5.98E-04 | -0.14 | 4.94E-01 |
| immunome | CD69⁺CD8Tₘₑₘ, pMAPKAPK2, basal | 5.90 | -0.27 | 1.90E-02 | -0.32 | 8.77E-02 |
| immunome | pDC, pSTAT1, IFNα | 5.05 | 0.51 | 3.95E-11 | 0.65 | 2.78E-04 |
| immunome | B cells, pMAPKAPK2, LPS | 2.96 | 0.31 | 1.38E-04 | -0.01 | 9.54E-01 |
| immunome | CD4Tem, pMAPKAPK2, basal | 1.69 | -0.19 | 1.85E-02 | -0.23 | 2.44E-01 |
| immunome | CD69⁺CD8Tₘₑₘ, pMAPKAPK2, IFNα | 1.28 | 0.19 | 7.57E-04 | 0.34 | 1.09E-01 |
| immunome | B cells (freq) | 1.11 | -0.14 | 9.30E-02 | -0.15 | 4.62E-01 |
| immunome | CCR5⁺CCR+CD4Tₑₘ, pNFκB, IL-2,4,6 | 1.10 | -0.19 | 2.26E-02 | 0.12 | 5.68E-01 |
| immunome | CCR⁺CCR2⁺CD4Tcm, IκB, basal | 0.97 | -0.28 | 5.87E-04 | -0.42 | 2.75E-02 |
| immunome | DC, pSTAT6, IFNα | 0.80 | -0.18 | 2.43E-02 | -0.20 | 3.08E-01 |
| immunome | DC, pMAPKAPK2, basal | 0.36 | -0.13 | 1.14E-01 | -0.24 | 2.25E-01 |

Goodness of fit of a pattern-fitting model [Akaike information criterion (AIC)] for the 45 most informative features of the integrated multiomic labor prediction model in the training cohort. The fits chosen were associated with p-values computed from the F-statistic to determine their relevance. Model index calculated from: -log10(pval)*abs(model coef). See also table S3. Related to FIG. 4.

TABLE 5

| Modality | Feature name | Model index | AIC degree 1 | AIC degree 2 | p-value | Class | Pattern |
|---|---|---|---|---|---|---|---|
| metabolome | 331.2264_8.4 (C₂₁H₃₀O₃17-OHP isomer) | 200.12 | 3425.85 | 3402.78 | 2.39e-31 | quadratic | acceleration/increase |
| metabolome | 331.2264_8.1 (C₂₁H₃₀O₃17-OHP isomer) | 161.21 | 2940.09 | 2930.63 | 1.68E-25 | quadratic | acceleration/increase |
| metabolome | 331.2265_8.9 (C₂₁H₃₀O₃17-OHP isomer) | 47.61 | 3326.09 | 3327.88 | 5.71E-18 | linear | constant |
| metabolome | 361.2017_7.1 (C₂₁H₃₀O₅ Cortisol isomer) | 27.84 | 3103.48 | 3103.91 | 1.24E-15 | linear | constant |
| metabolome | 415.3204_12 (C₂₇H₄₂O₃) | 16.43 | 3428.04 | 3429.92 | 4.71E-17 | linear | constant |
| metabolome | 151.0615_2.6 (1-Methylhypoxanthine) | 8.71 | 3678.81 | 3680.80 | 1.38E-11 | linear | constant |
| metabolome | 411.1844_8.7 (17-OH pregnenolone sulfate) | 7.73 | 3088.91 | 3059.97 | 9.52E-26 | quadratic | acceleration/increase |
| metabolome | 193.0618_5.3 (4-Aminohippuric acid) | 5.11 | 3158.65 | 3160.09 | 1.19E-09 | linear | constant |
| metabolome | 151.0612_6 (Arabitol, Xylitol) | 3.01 | 3669.95 | 3671.74 | 7.35E-04 | linear | constant |
| metabolome | 219.0774_6.3 (5-Hydroxytryptophan) | 2.42 | 3490.65 | 3492.62 | 2.13E-05 | linear | constant |
| metabolome | 236.0929_4.3 (N-Lactoylphenylalanine) | 2.17 | 3580.68 | 3582.19 | 3.96E-08 | linear | constant |
| metabolome | 397.205_10.6 (Pregnanolone sulfate) | 0.24 | 4275.53 | 4271.66 | 0.001 | quadratic | deceleration/decrease |
| proteome | IL-1R4 | 191.68 | 2567.88 | 2548.48 | 2.66E-26 | quadratic | acceleration/increase |
| proteome | Plexin-B2 (PLXB2) | 89.89 | 2537.32 | 2539.01 | 1.23E-20 | linear | constant |
| proteome | Discoidin domain receptor 1 (DDR1) | 68.74 | 2108.89 | 2110.75 | 1.66E-19 | linear | constant |
| proteome | Angiopoietin-2 | 52.63 | 1890.58 | 1892.08 | 1.41E-11 | linear | constant |
| proteome | Vascular Endothelial Growth Factor 121 | 45.08 | 2121.34 | 2122.95 | 9.02E-13 | linear | constant |
| proteome | Cystatin C | 38.77 | 2074.798 | 2074.796 | 1.34E-17 | quadratic | acceleration/increase |
| proteome | SLIT and NTRK-like protein 5 (SLTRK5) | 31.36 | 2713.88 | 2713.27 | 1.12E-10 | quadratic | deceleration/decrease |
| proteome | Secr. Leukocyte Peptidase Inhibitor (SLPI) | 24.90 | 2765.13 | 2761.58 | 2.07E-07 | quadratic | acceleration/increase |
| proteome | Activin A | 19.23 | 3218.71 | 3218.79 | 3.77E-15 | linear | constant |
| proteome | Antithrombin III | 10.27 | 3059.82 | 3056.07 | 3.18E-08 | quadratic | acceleration/decrease |
| proteome | Macrophage inhibitory cytokine-1 (MIC-1) | 9.96 | 3157.13 | 3158.62 | 1.01E-14 | linear | constant |
| proteome | Siglec-6 | 8.60 | 2952.64 | 2951.56 | 1.47E-12 | quadratic | acceleration/increase |
| proteome | urokinase-type Plasminogen Activator (uPA) | 8.09 | 2480.50 | 2482.13 | 1.23E-13 | linear | constant |
| proteome | Matrix Metalloproteinase (MMP) 12 | 5.87 | 2128.60 | 2129.72 | 2.76E-09 | linear | constant |
| proteome | Soluble tunica interna endothelial cell kinase (sTie)-2 | 4.91 | 2052.00 | 2051.45 | 9.27E-06 | quadratic | deceleration/decrease |
| proteome | LAG3 | 3.24 | 2536.34 | 2537.59 | 3.57E-06 | linear | constant |
| proteome | Endostatin | 1.73 | 2932.02 | 2932.58 | 8.54E-04 | linear | constant |
| proteome | GA733-1 protein | 0.27 | 1678.15 | 1674.65 | 5.49E-04 | quadratic | deceleration/decrease |
| immunome | CD69⁻CD56^{\|o}CD16⁺NK, pSTAT1, IFNα | 161.23 | 136.88 | -137.58 | 3.00E-17 | quadratic | deceleration/increase |
| immunome | Granulocytes (freq) | 18.20 | 901.11 | 902.06 | 3.87E-05 | linear | constant |
| immunome | CD69⁺CD56^{\|o}CD16⁺NK, pSTAT1, IFNα | 10.18 | 93.57 | -94.15 | 6.60E-15 | quadratic | deceleration/increase |
| immunome | CD62L⁺CD4T naive, pMAPKAPK2, IFNα | 8.92 | 796.26 | -794.41 | 5.15E-05 | quadratic | deceleration/increase |
| immunome | ncMC, pCREB, GM-CSF | 8.11 | 364.62 | -362.96 | 8.40E-04 | quadratic | acceleration/decrease |
| immunome | CD69⁺CD8Tₘₑₘ, pMAPKAPK2, basal | 5.90 | 662.37 | -665.48 | 7.53E-04 | quadratic | acceleration/decrease |
| immunome | pDC, pSTAT1, IFNα | 5.05 | 26.62 | 24.68 | 1.30E-11 | quadratic | deceleration/increase |
| immunome | B cells, pMAPKAPK2, LPS | 2.96 | 638.73 | -637.35 | 5.88E-05 | quadratic | deceleration/increase |
| immunome | CD4Tem, pMAPKAPK2, basal | 1.69 | 680.21 | -684.44 | 8.99E-04 | quadratic | acceleration/decrease |
| immunome | CD69⁺CD8Tₘₑₘ, pMAPKAPK2, IFNα | 1.28 | 810.97 | -809.10 | 1.75E-05 | quadratic | deceleration/increase |
| immunome | B cells (freq) | 1.11 | 670.77 | 672.74 | 5.22E-02 | linear | constant |
| immunome | CCR5⁺CCR⁺CD4Tem, pNFκB, IL-2,4,6 | 1.10 | 427.82 | -426.13 | 1.19E-02 | quadratic | deceleration/decrease |
| immunome | CCR⁺CCR2⁺CD4T_{cm}, IκB, basal | 0.97 | 418.89 | -418.56 | 5.74E-04 | quadratic | acceleration/decrease |
| immunome | DC, pSTAT6, IFNα | 0.80 | 45.62 | 40.80 | 1.26E-02 | quadratic | acceleration/decrease |
| immunome | DC, pMAPKAPK2, basal | 0.36 | 554.01 | -555.32 | 4.45E-02 | quadratic | acceleration/decrease |

What is claimed is:

1. A method for assessing time to onset of labor for an individual during pregnancy, the method comprising:
obtaining at two or more time-points during pregnancy a blood-based sample from the individual, comprising one or more features selected from: plasma proteins, metabolites and immune cells;
quantitating one or more of the features at the two or more time points;
determining whether changes in the features associated with onset of labor are present; and
providing an assessment of the individual’s time to onset of labor.

2. The method of claim 1, wherein treatment of the individual is made in accordance with the assessment.

3. The method of claim 1, wherein the two or more time-points are within second or third trimester of the pregnancy.

4. The method of claim 1, wherein the two or more timepoints are within the third trimester of the pregnancy.

5. The method of claim 1, wherein a sample is obtained at least 3, 4, 5, 6, 7, 8, 9, 10 time points.

6. The method of claim 1, wherein the trajectory of changes in a feature is determined.

7. The method of claim 1, wherein the quantitated features are selected from: 331.2264_8.4 (17-OHP/P4 derivative); 331.2264_8.1 (17-OHP/P4 derivative); 331.2265_8.9 (17-OHP/P4 derivative); 361.2017_7.1 (Cortisol); 415.3204_12 (C₂₇H₄₂O₃); 151.0615_2.6 (1-Methylhypoxanthine); 411.1844_8.7 (17-OH pregnenolone sulfate); 193.0618_5.3 (4-Aminohippuric acid); 151.0612_6 (Arabitol, Xylitol); 219.0774_6.3 (5-Hydroxytryptophan); 236.0929_4.3 (N-Lactoylphenylalanine); 397.205_10.6 (6 (Pregnanolone sulfate); IL-1R4; Plexin-B2 (PLXB2); Discoidin domain receptor 1 (DDR1); Angiopoietin-2; Vascular Endothelial Growth Factor 121; Cystatin C; SLIT and NTRK-like protein 5 (SLTRK5); Secr. Leukocyte Peptidase Inhibitor (SLPI); Activin A; Antithrombin III; Macrophage inhibitory cytokine-1 (MIC-1); Siglec-6; urokinase-type Plasminogen Activator (uPA); Matrix Metalloproteinase (MMP) 12; Soluble tunica interna endothelial cell kinase (sTie)-2; LAG3; Endostatin; GA733-1 protein; CD69⁻CD56^{lo}CD16⁺NK, pSTAT1, IFNα; Granulocytes (freq); CD69⁺CD56^{lo}CD16⁺NK, pSTAT1, IFNα; CD62L⁺CD4Tₙₐᵢᵥₑ, pMAPKAPK2, IFNα; ncMC, pCREB, GM-CSF; CD69⁺CD8Tₘₑₘ, pMAPKAPK2, basal; pDC, pSTAT1, IFNα; B cells, pMAPKAPK2, LPS; CD4Tₑₘ, pMAPKAPK2, basal; CD69⁺CD8Tₘₑₘ, pMAPKAPK2, IFNα; B cells (freq); CCR5⁺CCR⁺CD4Tₑₘ, pNFκB, IL-2,4,6; CCR⁺CCR2⁺CD4T_{cm}, IκB, basal; DC, pSTAT6, IFNα; DC, pMAPKAPK2, basal.

8. The method of claim 1, wherein the quantitated features comprise IL-1 receptor type 4 (IL-1R4); Activin-A; Sialic Acid Binding Ig Like Lectin (Siglec)-6; antithrombin III (ATIII); soluble tunica interna endothelial cell kinase (sTie)-2; PLXB2; DDR1; Angiopoietin-2; and vascular endothelial growth factor (VEGF)121.

9. The method of claim 1, wherein the quantitated features comprise: cortisol, Angiopoietin-2; granulocytes (frequency); isomers of 17-hydroxyprogesterone (17-OHP); 17-hydroxypregnenolone sulfate; IL-1 receptor type 4 (IL-1R4); dendritic cells pSTAT6 response to interferon α; soluble tunica interna endothelial cell kinase (sTie)-2; and CD69⁻CD56^{lo}CD16⁺NK cell pSTAT1 response to IFNα.

10. The method of claim 1, wherein the quantitated features comprise: phosphorylated (p)STAT1 signal in CD56dimCD16+ NK cells and the pSTAT6 signal in dendritic cells (DCs) in response to IFNα, the pP38, pERK and pCREB signals in classical monocytes (cMCs) in response to LPS and GM-CSF, and the pCREB response in non-classical monocytes (ncMCs) in response to GM-CSF.

11. The method of claim 1, wherein from 5 to 15 features are quantitated.

12. The method of claim 1, wherein from 5 to 10 features are quantitated.

13. The method of claim 1, wherein the quantitated features comprise: isomers of 17-hydroxyprogesterone (17-OHP); and 17-hydroxypregnenolone sulfate.

14. The method of claim 1, wherein the quantitated features comprise features from metabolome, proteome and immunome.

15. The method of claim 1, wherein multiple features are integrated in a multivariate model.

16. The method of claim 15, wherein a stacked generalization (SG) algorithm is applied to a dataset of feature quantitation measurements for an integrated model, where linear regression models a first individually built for each feature dataset, then integrated into a single model by SG.

17. The method of claim 1, wherein immunome features are quantitated by flow cytometry.

18. The method of claim 1, wherein metabolome features are assessed by mass spectroscopy.

19. The method of claim 1, wherein proteome features are assessed by affinity binding.
